(19) United States
(12) Patent Application Publication
(10) Pub. No.: US 2010/0130815 A1
(43) Pub. Date: May 27, 2010

(54) INTRAURETHRAL AND EXTRAURETHRAL APPARATUS

(75) Inventors: Yossi Gross, Moshav Mazor (IL); Eliahu Eliachar, Haifa (IL); Nir Lilach, Kfar Yehoshua (IL); Ram Grossfeld, Haifa (IL); Dan Sade, Beit Alfa (IL)

Correspondence Address: DARBY & DARBY P.C., P.O. BOX 770, Church Street Station, New York, NY, 10008-0770, US

(73) Assignee: PROSTAPLANT LTD., Herzliya (IL)

(21) Appl. No.: 12/620,784

(22) Filed: Nov. 18, 2009

Related U.S. Application Data
(63) Continuation-in-part of application No. PCT/IL08/00677, filed on May 18, 2008.
(60) Provisional application No. 60/930,705, filed on May 18, 2007.
(60) Provisional application No. 61/200,372, filed on Nov. 26, 2008.

(51) Int. Cl. A61F 2/02

(52) U.S. Cl. 600/30

(57) ABSTRACT

A method is provided, including distally advancing an implant through a urethra of a patient until the implant emerges in a bladder of the patient, and facilitating expanding of a pre-operative perimeter of a portion of the urethra to a post-operative perimeter of the portion of the urethra that is larger than the pre-operative perimeter by proximally retracting the implant and implanting the implant in prostate tissue surrounding the urethra. Other embodiments are also described.

### CROSS-REFERENCES TO RELATED APPLICATIONS

The present application:

(a) is a continuation-in-part of PCT Patent Application PCT/IL08/00677 to Gross et al., entitled, “Prostate implant and methods for insertion and extraction thereof,” filed May 18, 2008 which claims priority from U.S. Provisional Patent Application 60/930,705 to Gross et al., entitled, “Prostate implant and methods for insertion and extraction thereof,” filed May 18, 2007; and

(b) claims priority from U.S. Provisional Patent Application 61/200,372 to Gross et al., entitled, “Intraurethral and extraurethral apparatus,” filed Nov. 26, 2008.

All of the above applications are incorporated herein by reference.

### FIELD OF THE INVENTION

Some applications of the present invention relate generally to implants and delivery tools therefor. Specifically, some applications of the present invention relate to an implant that is placed around or within a body lumen, such as but not limited to, a transurethrally implantable prostatic implant for treatment of benign prostatic hyperplasia (BPH).

### BACKGROUND OF THE INVENTION

Benign prostatic hyperplasia (BPH) is a condition wherein a benign (non-cancerous) tumor with nodules enlarges the prostate gland. Although the growth is non-cancerous, the internal lobes of the prostate slowly enlarge and progressively occlude the urethral lumen. Severe BPH can cause serious problems over time: Urine retention and strain on the bladder can lead to urinary tract infections, bladder or kidney damage, bladder stones, and incontinence.

U.S. Pat. No. 7,004,965 to Gross, which is incorporated herein by reference, describes an implant system including a transurethral prostatic implant positioned in a prostate and including a lumen with an inner perimeter that surrounds an outer perimeter of a urethra at the prostate. The implant system includes a delivery tool including a shaft having a distal portion and an implant-holding portion proximal to the distal portion, the distal portion being sized for entry into a urethra, and the implant-holding portion being thicker than the distal portion, and an implant positioned on the implant-holding portion.

U.S. Pat. No. 5,601,591 to Edwards et al., describes a stent for introduction into a portion of a urethra in a body of a patient. The stent includes a longitudinally-extending body made from a material adapted for absorption by the body of the patient. The longitudinally-extending body has an expanded condition in which the body has a predetermined diameter greater than the diameter of the portion of the urethra extending through the prostate. The longitudinally-extending body is formed with a plurality of coils along the length thereof adapted to engage the wall of the urethra when the longitudinally-extending body is in the expanded condition. The longitudinally-extending body is provided with spaces between the coils to permit the wall of the urethra to extend therein and serve to anchor the longitudinally-extending body to the wall.

U.S. Pat. No. 6,517,566 to Hovland et al., describes a permanent implanted support for e.g. the urethral neck of the bladder, generally preventing urinary leakage caused by transmission of intra-abdominal pressure pulse waves. The support is implanted in a straightforward manner without the significant complexity and invasiveness associated with known surgical techniques. Pelvic trauma is dramatically reduced. The support can be used in treatment of stress incontinence, and other types of incontinence, in both males and females.

U.S. Pat. No. 6,991,647 to Jadhav, describes a bio-compatible and bioresorbable stent that is intended to restore or maintain patency following surgical procedures, traumatic injury or stricture formation. The stent composes a blend of at least two polymers that is either extruded as a monofilament then woven into a braid-like embodiment, or injection molded or extruded as a tube with fenestrations in the wall. Methods for manufacturing the stent are also disclosed.

U.S. Pat. No. 7,104,949 to Anderson et al., describes a minimally invasive surgical instrument for placing an implantable article about a tubular tissue structure. The surgical instrument is described as being useful for treating urological disorders such as incontinence. Surgical methods using the novel instrument are also described.

US Patent Application Publication 2004/0181287 to Gellman, describes a stent for treatment of a body lumen through which a flow is effected on either side of a sphincter, said stent comprising one or more windings and having an inner core substantially covered by an outer core and including a first segment, a second segment, and a connecting member disposed between the segments. When the stent is positioned within a patient's urinary system, the first segment and second segments are described as being located on either side of the external sphincter to inhibit migration of the stent while not interfering with the normal functioning of the sphincter. The outer coating is described as comprising an absorbable material that provides temporary structural support to the stent. After absorption of substantially all the outer coating of the stent, the remaining relatively compliant inner core facilitates removal by the patient by pulling a portion of the stent that extends outside the patient's body for this purpose.

US Patent Application Publication 2006/0276871 to Lamson et al., describes devices, systems and methods for compressing, cutting, incising, reconfiguring, remodeling, attaching, repositioning, supporting, dislocating or altering the composition of tissues or anatomical structures to alter their positional or force relationship to other tissues or anatomical structures. In some applications, the invention may be used to used to improve patency or fluid flow through a body lumen or cavity (e.g., to limit constriction of the urethra by an enlarged prostate gland).

The following patents and patent application publications, may be of interest:

PCT Publication WO 02/058577 to Cionta et al.

U.S. Pat. No. 4,978,323 to Freedman

U.S. Pat. No. 5,160,341 to Brenneman et al.

U.S. Pat. No. 5,776,142 to Gunderson

U.S. Pat. No. 6,119,045 to Bolmsjo

U.S. Pat. No. 6,231,516 to Keilman

U.S. Pat. No. 6,258,094 to Nicholson et al.

U.S. Pat. No. 6,280,465 to Cryer

U.S. Pat. No. 6,679,851 to Burbank et al.

U.S. Pat. No. 6,702,846 to Mikus et al.

U.S. Pat. No. 6,709,452 to Valimaa et al.

U.S. Pat. No. 7,175,589 to Deem et al.

US Patent Application Publication 2002/0177904 to Huxel et al.

US Patent Application Publication 2003/0144658 to Schwartz et al.

US Patent Application Publication 2004/0133263 to Dusbabek et al.

US Patent Application Publication 2004/0254520 to Porteous et al.

US Patent Application Publication 2005/0216074 to Sahatjian et al.

US Patent Application Publication 2006/0095058 to Sivan et al.

US Patent Application Publication 2006/0106109 to Burbank et al.

US Patent Application Publication 2006/0173517 to Gross

US Patent Application Publication 2007/0106233 to Huang et al.

US Patent Application Publication 2008-0039889 to Lamson et al.

### SUMMARY OF EMBODIMENTS OF THE INVENTION

In some embodiments of the present invention, a system for treating urethral constriction at the prostate comprises at least one transurethrally implantable prostatic implant and a delivery tool therefor. The delivery tool is advanced into a constricted urethra of a patient. Typically, the implant is removably coupled to the delivery tool at a distal site of the tool. The delivery tool functions to advance the implant distally through the urethra of the patient. In some embodiments, the implant is advanced until the implant emerges at a distal end of the urethra and into a bladder of the patient. (In this context, in the specification and in the claims, “proximal” means closer to the orifice through which the tool is originally placed into the urinary tract, and “distal” means further from this orifice.)

Typically, the implant comprises a coiled implant comprising at least one coil, which is disposed in a compressed state during transurethral advancement thereof. In some embodiments, the implant comprises a radially-expandable implant configured to expand prior to implantation of the implant in the vicinity of the prostate of the patient. In some embodiments, the implant comprises a rigid material, e.g., stainless steel, and is configured for advancement through the urethra in a compressed state thereof in which the implant had a narrow diameter. Prior to implantation of the implant, the implant is expanded to assume a larger diameter using a mechanical device, e.g., a balloon, a stent, or a basket wire, that is disposed in a lumen of the compressed implant.

In some embodiments, the implant is shaped to define an implant lumen that is configured to surround an outer circumference of the urethra as the implant is implanted within the prostate. For embodiments in which the implant is implanted around the urethra from within the bladder, a pointed tip at a proximal end of the implant enables the implant to puncture and penetrate into the prostatic tissue surrounding the urethra of the patient. Rotation of a portion of the delivery tool about a longitudinal axis thereof moves the implant proximally while corkscrewing the implant into the prostatic tissue and thereby around the urethra in order to maintain an expanded diameter of the pathologically constricted urethra. Typically, the implant is configured to reside chronically in the prostate of the patient.

In some embodiments, the implant is corkscrewed around the urethra of the patient while the implant is disposed in the urethra. In such an embodiment, the implant comprises a pointed proximal tip which punctures tissue of the urethra and facilitates the proximal corkscrewing of the implant around the urethra. Alternatively, the implant comprises a pointed distal tip which punctures tissue of the urethra and facilitates the distal corkscrewing of the implant around the urethra.

In either embodiment, following implantation, the implant radially supports the prostatic tissue and maintains an expanded diameter of the pathologically constricted urethra. Typically, the implant is configured to reside chronically in the prostate of the patient.

In some embodiments of the present invention, the at least one implant comprises two or more coiled implants which, when implanted within the tissue, are configured to be disposed in a relative spatial configuration in which the implants are concentrically disposed and have opposing rotational directions, i.e., one implant is left-handed and the other is right-handed. In some embodiments, respective ends of the implants are rotationally offset by a given angle with respect to each other. The two or more implants are implanted substantially at the same time. During the implantation, each implant is rotated in a direction corresponding to the rotational direction of the implant. Additionally, in response to the rotational force of a first one of the implants in a given direction, the prostatic tissue is pulled in the given direction. By implanting two implants having opposing rotational directions, the opposing rotational force applied to the tissue by the second one of the implants balances the rotational force applied to the tissue by the first implant. Thus, the pulling of the prostatic tissue in a given direction is reduced.

In some embodiments of the present invention, the at least one implant comprises two or more coiled implants which, when implanted within the tissue, are configured to be disposed in a relative spatial configuration in which the implants are coaxially disposed and rotationally offset by a given angle with respect to each other. Additionally, when positioned in the relative spatial configuration, at least a portion of each of the implants overlap longitudinally. Typically, the implants longitudinally essentially entirely overlap each other.

In some embodiments, a plurality of distinct coiled implants are implanted around the urethra. In some embodiments, a plurality of distinct curved needles are implanted around the urethra.

In some embodiments, a conically-shaped coiled implant is implanted in the prostate and functions as a scaffold for advancement therethrough and support of a plurality of longitudinal rods. The rods are advanced around the urethra in a manner in which the rods are disposed circumferentially around the urethra and maintain an open state of the constricted urethra. For some applications, the rods are implanted prior to the implanting of the coiled implants with respect to the rods. In such an embodiment, the implant is not necessarily conic, and may comprise any coil-shaped implant.

Typically, one or more implants are disposed within a lumen of a delivery tool comprising a deflectable distal tip which is controllable by the operating physician to be steerable radially away from a longitudinal axis of the delivery tool. In response to deflecting the distal tip of the tool, the distal tip pushes the wall of the urethra which compresses tissue outside of the urethra, i.e., prostate tissue, and consequently the perimeter of the urethra at the prostate expands. The delivery tool then delivers an implant in the portion of the tissue of the prostate that has been compressed, and the implant functions to maintain the tissue in a compressed state upon withdrawal of the delivery tool from the urethra.

For some applications, a plurality of implants are designated for implantation in respective portions of the prostate. For example, a plurality of implants may be implanted adjacent to (e.g., around) the urethra in a single transverse sectional plane of the prostate. For some applications, a plurality of implants are sequentially disposed along parallel planes of the prostate along a longitudinal axis of the urethra. Typically, the implants comprise coiled implants which are implanted in a manner in which a longitudinal axis of the implant is disposed at a non-zero angle, e.g., 90 degrees, with respect to the longitudinal axis of the urethra.

For some applications, each coiled implant, which is implanted at the non-zero angle with respect to the urethra, is delivered to the prostate tissue in an expanded state thereof along a longitudinal axis of the implant. Upon implantation, the implant compresses to return to its unexpanded resting state, and thereby compresses tissue radially with respect to the longitudinal axis of the urethra. For some applications, since these coiled implants are made to pull tissue in response to a tension force, these coiled implants are typically implanted in the prostate without first pushing the tissue.

There is therefore provided, in accordance with an embodiment of the present invention apparatus, including:

an implant; and

a delivery tool, removably coupled to the implant, the tool configured to:
advance the implant distally through a urethra of a patient until the implant emerges at a distal end of the urethra into a bladder of the patient, and
subsequently, facilitate expansion of the urethra by retracting the implant and by the retracting, implant the implant around the urethra in tissue of a prostate of the patient.

In an embodiment, the implant includes a low-friction coating.

In an embodiment a surface of the implant includes a polished surface configured to reduce friction of the implant during implantation.

In an embodiment the implant is radially-expandable, and configured to expand upon emergence into the bladder.

In an embodiment the implant includes a transurethrally-implantable prostatic implant configured to be positionable in the prostate of the patient, and the implant is shaped so as to define an implant lumen that surrounds an outer circumference of the urethra upon implantation.

In an embodiment the implant is shaped to define an implant lumen having an inner diameter of at least 2.5 mm.

In an embodiment the implant is shaped to define an implant lumen having an inner diameter of between 2.5 mm and 15 mm.

In an embodiment the implant is configured to prevent stenosis of the urethra.

In an embodiment the implant is configured to treat benign prostate hyperplasia.

In an embodiment the delivery tool is shaped to define a delivery tool lumen for passing an imaging device therethrough.

In an embodiment the apparatus includes an imaging device configured to guide the retraction of the implant.

In an embodiment the delivery tool includes a rotating element configured to corkscrew the implant into the tissue during the retracting of the implant.

In an embodiment the delivery tool includes a rotating element configured to corkscrew the implant into the tissue by rotation about a longitudinal axis of the delivery tool.

In an embodiment the implant includes a flexible, biocompatible material selected from the group consisting of: nitinol and silicone.

In an embodiment the apparatus includes a needle coupled to a proximal end of the implant.

In an embodiment the needle includes a rigid, biocompatible material configured to puncture tissue of the patient.

In an embodiment the needle includes stainless steel.

In an embodiment the implant includes at least one rod, and the delivery tool is configured to implant the rod in tissue of the prostate at an angle that is less than 90 degrees with respect to a longitudinal axis of the urethra.

In an embodiment the apparatus includes at least one coiled implant, and the delivery tool is configured to implant the implant in tissue of the prostate in a manner in which the at least one coiled implant is couplable to the rod at at least a portion of the coiled implant.

In an embodiment, the rod has a longitudinal axis that is less than 90 degrees with respect to the longitudinal axis of the urethra, and the coiled implant is implantable at a non-zero angle with respect to the longitudinal axis of the rod.

In an embodiment, the implant includes a coiled implant including at least one coil.

In an embodiment, the delivery tool is configured to corkscrew the coiled implant into the prostate while retracting the implant.

In an embodiment, the coiled implant is configured to corkscrew into the prostate of the patient.

In an embodiment, the coil includes a conically-shaped coiled implant.

In an embodiment, a proximal coil of the conically-shaped coiled implant has a diameter that is larger than a diameter of a distal coil of the conically-shaped coiled implant.

In an embodiment, the coiled implant is configured to corkscrew into tissue of the patient.

In an embodiment, the coiled implant is shaped to define a proximal pointed end configured to puncture the tissue.

In an embodiment, the delivery tool is configured to implant the implant around the urethra by corkscrewing the coiled implant into the tissue while retracting the implant.

In an embodiment, the implant is shaped to define at least one slit configured for engaging of the delivery tool thereto.

In an embodiment, the implant includes a coiled implant.

In an embodiment, the implant is shaped to provide a proximal slit and a distal slit.

In an embodiment, the delivery tool includes a proximal locking mechanism and a distal locking mechanism, the proximal locking mechanism is configured to engage the proximal slit of the implant, and the distal locking mechanism is configured to engage the distal slit of the implant.

In an embodiment, the proximal and distal locking mechanisms are configured to maintain the implant in a compressed state thereof during the advancement of the implant into the bladder of the patient.

In an embodiment, the implant is configured to expand radially following a disengagement of the proximal locking mechanism therefrom.

In an embodiment, the implant is shaped to define a helical implant, and the apparatus includes a sheath shaped to define a hollow lumen helically surrounding the helical implant.

In an embodiment the apparatus includes, an ablation tool configured to be slidably advanced through the lumen of the sheath, and the sheath is shaped to define at least one hole at a proximal end thereof configured for advancement therethrough of at least a portion of the ablation tool.

In an embodiment the apparatus includes, a flexible tube coupled to a portion of the sheath, the tube being configured to facilitate passage of a fluid through the lumen of the sheath, and the sheath is shaped to define one or more holes configured for passage of the fluid externally to the implant.

In an embodiment, the implant includes a hollow, helical implant shaped to define a helical lumen thereof.

In an embodiment the apparatus includes, an ablation tool configured to be slidably advanced through the lumen of the implant, and the implant is shaped to define at least one hole at a proximal end thereof configured for advancement therethrough of at least a portion of the ablation tool.

In an embodiment the apparatus includes, a flexible tube coupled to a portion of the helical implant, the tube being configured to facilitate passage of a fluid through the lumen of the implant, and the implant is shaped to define one or more holes configured for passage of the fluid externally to the implant.

In an embodiment, the implant defines a first implant, and the apparatus includes a second implant.

In an embodiment, the first and second implants include respective first and second helical implants.

In an embodiment, the at least first and second helical implants are configured to assume respective longitudinal positions and are configured to be disposed in a relative spatial configuration in which:

the first and second helical implants are disposed coaxially,

the first and second helical implants are rotationally offset, and

the respective longitudinal positions of the first and second helical implants overlap at least in part.

In an embodiment, the first and second implants have the same diameter.

In an embodiment, the first and second helical implants each have a first pitch, and, when disposed in the relative spatial configuration, the first and second implants define an effective second pitch which is less than half the first pitch.

In an embodiment, the delivery tool is configured to implant the first and second helical implants sequentially and position the first and second helical implants in the relative spatial configuration thereof.

In an embodiment, the first and second helical implants are configured to be coupled to the delivery tool in the relative spatial configuration.

In an embodiment, the first and second helical implants are configured to be simultaneously implanted around the urethra of the patient.

In an embodiment, the at least first and second helical implants include respective transurethrally-implantable prostatic implants configured to be positionable in the prostate of the patient, and the first and second implants are shaped to define respective implant lumens that surround an outer circumference of the urethra upon implantation.

In an embodiment, the first and second helical implants are shaped to define respective proximal pointed ends configured to puncture the tissue.

There is further provided, in accordance with an embodiment of the present invention, a method, including:

distally advancing an implant through a urethra of a patient until the implant emerges in a bladder of the patient; and

facilitating expanding of a pre-operative perimeter of a portion of the urethra to a post-operative perimeter of the portion of the urethra that is larger than the pre-operative perimeter by proximally retracting the implant and implanting the implant in prostate tissue surrounding the urethra.

In an embodiment, expanding the pre-operative perimeter includes treating benign prostate hyperplasia.

In an embodiment, advancing the implant includes advancing a coiled implant defining an inner lumen thereof, and implanting the implant includes surrounding a portion of the urethra by the inner lumen of the coiled implant.

In an embodiment, facilitating the expanding of the pre-operative perimeter of a portion of the urethra includes the surrounding of the portion of the urethra by the inner lumen of the coiled implant.

In an embodiment, advancing the implant includes advancing at least one rod through the urethra, and implanting the implant includes retracting the rod into the prostate tissue at an angle that is less than 90 degrees with respect to a longitudinal axis of the urethra.

In an embodiment the method further includes:

distally advancing at least one coiled implant through the urethra of the patient;

further facilitating expanding of the pre-operative perimeter of the portion of the urethra by implanting the at least one coiled implant in the prostate tissue; and

facilitating coupling to the rod at least a portion of the coiled implant.

In an embodiment, the rod has a longitudinal axis that is less than 90 degrees with respect to the longitudinal axis of the urethra, and implanting the at least one coiled implant includes implanting the at least one coiled implant at a non-zero angle with respect to the longitudinal axis of the rod.

In an embodiment, the implant includes a radially-expandable implant, and advancing the implant into the bladder includes facilitating the expansion of the implant within the bladder of the patient.

In an embodiment, the implant includes a conically-shaped coiled implant in which a diameter of a proximal coil thereof is larger than a diameter of a distal coil thereof, and implanting the implant includes implanting the conically-shaped coiled implant in the prostate tissue of the patient.

In an embodiment, the method includes reversibly coupling the implant to a delivery tool, and advancing the implant includes advancing the delivery tool, when it is reversibly coupled to the implant, through the urethra of the patient.

In an embodiment, implanting the implant includes decoupling the implant from the delivery tool.

In an embodiment, proximally retracting the implant includes corkscrewing the implant into the prostate tissue by rotating at least a portion of the delivery tool.

In an embodiment, implanting the implant includes corkscrewing the implant into the prostate tissue by rotating at least a portion of the delivery tool.

In an embodiment, the method includes imaging via an imaging device coupled to the delivery tool.

In an embodiment, imaging includes examining the bladder of the patient via the imaging device, prior to the advancing of the implant through the urethra, by imaging a vicinity of a neck of the bladder of the patient.

In an embodiment, imaging includes imaging the implanting of the implant in the tissue surrounding the urethra of the patient.

In an embodiment, distally advancing the implant includes distally advancing at least first and second implants through the urethra of the patient, and implanting the implant includes implanting the at least first and second implants in the prostate tissue.

In an embodiment, implanting the at least first and second implants in tissue surrounding the urethra implant includes corkscrewing the first and second implant into the prostate tissue.

In an embodiment the method includes:

reversibly coupling the first implant to a delivery tool; and

reversibly coupling the second implant to the delivery tool, and

advancing the first and second implants includes advancing the first and second implants through the urethra of the patient by the delivery tool.

In an embodiment, implanting the first and second implants includes decoupling the first and second implants from the delivery tool.

In an embodiment, proximally retracting the implant includes corkscrewing the first and second implants into the tissue by rotating at least a portion of the delivery tool.

In an embodiment, implanting the first and second implants includes implanting first and second implants in respective longitudinal positions thereof in a relative spatial configuration in which:

the first and second helical implants are disposed coaxially,

the first and second helical implants are rotationally offset, and

the respective longitudinal positions of the first and second helical implants overlap at least in part.

In an embodiment, reversibly coupling the first and second implants to the delivery tool includes reversibly coupling to the delivery tool the first and second implants in the relative spatial configuration thereof, and advancing the first and second implants includes simultaneously advancing the first and second implants through the urethra of the patient.

In an embodiment, implanting the first and second implants in the relative spatial configuration thereof includes:

during a first period:
reversibly coupling the first implant to the delivery tool,
advancing the delivery tool, when it is reversibly coupled to the first implant, through the urethra of the patient, and
implanting the first implant in tissue surrounding the urethra by proximally retracting the first implant through a first opening created by the first implant, and

during a second period subsequent to the first period:
reversibly coupling the second implant to the delivery tool,
advancing the delivery tool, when it is reversibly coupled to the second implant, through the urethra of the patient, and
implanting the second implant in tissue surrounding the urethra by proximally retracting the second implant through a second opening created by the second implant, and the second opening is rotationally offset from the first opening with respect to a longitudinal axis of the urethra.

There is additionally provided, in accordance with an embodiment of the present invention, a method, including:

at a first time, implanting an implant around a lumen of a patient by:

advancing the implant distally through the lumen until the implant emerges at a distal end of the lumen into a cavity, and

subsequently, implanting the implant around the lumen by proximally retracting the implant; and

at a second time, extracting the implant from around the lumen by:
moving the implant distally by rotating the implant, and
subsequently, pulling the implant proximally through the lumen.

In an embodiment, implanting the implant around the lumen includes rotating the implant in a first direction thereof, and extracting the implant includes rotating the implant in a reverse direction to the first direction.

In an embodiment, the implant includes a radially-expandable implant, and advancing the implant includes allowing the expansion of the implant within the cavity.

In an embodiment, extracting the implant includes:
clamping a distal portion of the implant and moving the implant distally by rotating the implant; and
clamping a proximal portion of the implant.

In an embodiment, the lumen includes a urethra of the patient and pulling the implant includes pulling the implant through the urethra.

In an embodiment, rotating the implant includes extracting the implant from a prostate of the patient.

There is yet further provided, in accordance with an embodiment of the present invention, apparatus, including:

at least first and second helical implants configured to assume respective longitudinal positions and to be disposed in a relative spatial configuration in which:

the first and second helical implants are disposed coaxially,

the first and second helical implants are rotationally offset, and

the respective longitudinal positions of the first and second helical implants overlap at least in part; and

a delivery tool, configured to be reversibly coupled to the at least first and second helical implants, the tool configured to:

advance the at least first and second implants distally through a body lumen of a patient until the first and second implants emerge at a distal end of the body lumen into a body cavity of the patient, and
implant the at least first and second implants in the relative spatial configuration thereof around the body lumen by retracting the first and second implants.

In an embodiment, the first and second implants have the same diameter.

In an embodiment, the first and second implants include low-friction coatings.

In an embodiment, the first and second implants are radially expandable, and configured to expand upon emergence into the body cavity.

In an embodiment, the body lumen includes a urethra, and the first and second implants are configured to be implanted around the urethra.

In an embodiment, the first and second implants include respective transurethrally-implantable prostatic implants configured to be positionable in a prostate of the patient, and the body lumen includes a urethra of the patient, the implants being shaped to define respective implant lumens that surround an outer circumference of the urethra upon implantation.

In an embodiment, the first and second implants are shaped to define respective implant lumens that surround an outer circumference of the lumen upon implantation.

In an embodiment, the first and second implants are shaped to define respective inner diameters of at least 2.5 mm.

In an embodiment, the first and second implants are shaped to define respective inner diameters of between 2.5 mm and 15 mm.

In an embodiment, the first and second helical implants each have a first pitch, and, when disposed in the relative spatial configuration, the first and second implants define an effective second pitch which is less than half the first pitch.

In an embodiment, the delivery tool is configured to implant the first and second helical implants sequentially and position the first and second helical implants in the relative spatial configuration thereof.

In an embodiment, the first and second helical implants are shaped to define respective proximal pointed ends configured to puncture the tissue.

In an embodiment, the first and second helical implants are configured to be coupled to the delivery tool in the relative spatial configuration.

In an embodiment, the first and second helical implants are configured to be simultaneously implanted around the body lumen of the patient.

There is still further provided, in accordance with an embodiment of the present invention, a method, including:

creating a first opening in tissue of a patient by puncturing the tissue; advancing through the first opening a first helical implant to a first longitudinal position;

creating a second opening in tissue of the patient by puncturing the tissue, the second opening being rotationally offset from the first opening with respect to a longitudinal axis of the first helical implant when it has been advanced through the first opening; and

advancing through the second opening a second helical implant to a second longitudinal position, in which:
the first and second helical implants are disposed coaxially,
the first and second helical implants are rotationally offset with respect to each other, and
respective longitudinal positions of the first and second helical implants overlap at least in part.

In an embodiment, advancing through the first opening the first helical implant to the first longitudinal position includes corkscrewing the first helical implant into the tissue, and advancing through the second opening the second helical implant to the second longitudinal position includes corkscrewing the second helical implant into the tissue.

In an embodiment, the tissue includes a prostate of the patient, and advancing the first and second helical implants includes corkscrewing the first and second helical implants into the prostate.

In an embodiment, the method includes:

distally advancing the first and second helical implants through a body lumen of a patient until the first and second implants emerge in a body cavity of the patient, and:

advancing through the first opening the first helical implant to the first longitudinal position includes implanting the first implant in tissue surrounding the body lumen by proximally retracting the first implant, and

advancing through the second opening the second helical implant to the second longitudinal position includes implanting the second implant in tissue surrounding the body lumen by proximally retracting the second implant.

In an embodiment, the method includes:

reversibly coupling the first implant to a delivery tool; and

reversibly coupling the second implant to the delivery tool, and

distally advancing the first and second implants includes distally advancing the first and second implants through the body lumen of the patient by the delivery tool.

In an embodiment, implanting the first and second implants includes decoupling the first and second implants from the delivery tool.

In an embodiment, proximally retracting the implants includes corkscrewing the first and second implants into the tissue by rotating at least a portion of the delivery tool.

In an embodiment, reversibly coupling the first and second implants to the delivery tool includes reversibly coupling to the delivery tool the first and second implants in the relative spatial configuration thereof, and advancing the first and second implants includes simultaneously advancing the first and second implants through the body lumen of the patient.

In an embodiment, implanting the first and second implants in the relative spatial configuration thereof includes:

during a first period:
reversibly coupling the first implant to the delivery tool,
advancing the delivery tool, when it is reversibly coupled to the first implant, through the body lumen of the patient, and
implanting the first implant in tissue surrounding the body lumen by proximally retracting the first implant through a first opening created by the first implant, and

during a second period subsequent to the first period:
reversibly coupling the second implant to the delivery tool,
advancing the delivery tool, when it is reversibly coupled to the second implant, through the body lumen of the patient, and
implanting the second implant in tissue surrounding the body lumen by proximally retracting the second implant through a second opening created by the second implant, and the second opening is rotationally offset from the first opening with respect to a longitudinal axis of the body lumen.

There is yet additionally provided, in accordance with an embodiment of the present invention, apparatus, including:

a helical implant; and

a sheath, helically surrounding the implant, the sheath shaped to define one or more holes.

In an embodiment, the sheath is shaped to define three or more holes.

In an embodiment, the helical implant is shaped to define an inner lumen having a diameter thereof that is between 2.5 mm and 15 mm.

In an embodiment, the sheath tightly surrounds the helical implant.

In an embodiment the apparatus includes:

a lubricant; and

a pressure source configured to push the lubricant (a) from within a space between the helical implant and the sheath, (b) through the one or more holes, (c) to outside of the sheath.

There is also provided, in accordance with an embodiment of the present invention, apparatus, including:

a helical implant having a wall shaped to define a plurality of holes, the helical implant shaped to define a helical lumen thereof;

a lubricant, disposed within the lumen; and

a pressure source, configured to push the lubricant through the plurality of holes.

In an embodiment, the pressure source includes a syringe.

In an embodiment, the helical implant is shaped to define an inner lumen having a diameter thereof that is between 2.5 mm and 15 mm.

There is additionally provided, in accordance with an embodiment of the present invention, apparatus, including:

first and second coiled implants, an outer diameter of the second implant being smaller than an inner diameter of the first implant, one of the coiled implants being right-handed and one of the coiled implants being left-handed; and

a delivery tool, reversibly couplable to the first and second coiled implants, the tool being configured to facilitate implantation of the first and second implants around a body lumen of a patient.

In an embodiment, the second implant is configured to be disposed concentrically with respect to the first implant.

In an embodiment, the first and second implants includes low-friction coatings.

In an embodiment, a respective surface of each of the first and second implants includes a polished surface configured to reduce friction of the implants during implantation.

In an embodiment, the first and second implants are radially-expandable, and are configured to expand prior to the implantation of the first and second implants around the body lumen of the patient.

In an embodiment, the body lumen includes a urethra, and the first and second implants are configured to be implanted around the urethra.

In an embodiment, the first and second implants include respective transurethrally-implantable prostatic first and second implants configured to be positionable in a prostate of the patient, and the body lumen includes a urethra of the patient, the implants each being shaped to define a respective implant lumen that is configured to surround an outer circumference of the urethra upon implantation.

In an embodiment, the first and second implants are shaped to define respective implant lumens that are configured to surround an outer circumference of the lumen upon implantation.

In an embodiment, the first and second implants are each shaped to define respective inner diameters of at least 2.5 mm.

In an embodiment, the first and second implants are each shaped to define respective inner diameters of between 2.5 mm and 15 mm.

In an embodiment, the first and second implants are each shaped to define respective proximal pointed ends configured to puncture the tissue.

In an embodiment, the first and second implants are each shaped to define respective distal pointed ends configured to puncture the tissue.

In an embodiment, the first and second implants are configured to prevent stenosis of the body lumen.

In an embodiment the apparatus includes, an imaging device configured to guide the implantation of the implants.

In an embodiment, the delivery tool is shaped to define a delivery tool lumen for passing an imaging device therethrough.

In an embodiment, the delivery tool includes a rotating element configured to corkscrew the implants into the tissue during the implantation thereof.

In an embodiment, the delivery tool includes a rotating element configured to corkscrew the implants into the tissue by rotation about a longitudinal axis of the delivery tool.

In an embodiment, each one of the first and second implants is shaped to define at least two conically-shaped portions.

In an embodiment the apparatus includes, a motor coupled to the apparatus, the motor being configured to facilitate implantation of the first and second implants around the body lumen.

In an embodiment, the motor is coupled to the delivery tool.

In an embodiment the apparatus includes, first and second motors, the first motor is coupled to the first implant and the second motor is coupled to the second implant.

In an embodiment, the motor includes an ultrasound transducer configured to create vibrations in the implants in response to vibrations effected by the ultrasound transducer.

In an embodiment, the motor includes a vibrator configured to create vibrations in the implants in response to vibrations effected by the vibrator.

In an embodiment, the motor is configured to control the implantation of the implants around the body lumen by cycling between:

(a) facilitating advancement, by a first number of degrees, of the implants in their respective first rotational directions through tissue of the body lumen, and

(b) facilitating retracting, by a second number of degrees, of the implants in a second rotational direction that is opposite the first direction.

In an embodiment:

the first and second implants includes first and second wires, respectively, the first and second wires being shaped to define the respective first and second implants,

the first wire has a width that is larger than the second wire, and

the first implant has a diameter that is larger than the second implant.

In an embodiment, the first and second wires are shaped to define a shape in a cross-section thereof, the shape being selected from the group consisting of: a triangle, a square, a diamond, a circle, and an ellipse.

In an embodiment, the first and second implants are configured to be coupled to the delivery tool in a configuration in which the second implant is disposed concentrically with respect to the first implant.

In an embodiment, the delivery tool is configured to facilitate simultaneous implantation of the first and second helical implants around the body lumen of the patient.

In an embodiment, the first and second helical implants are configured to be sequentially implanted around the body lumen of the patient, and following the implantation of the first and second implants, the first and second implants are configured to be disposed concentrically with respect to each other.

In an embodiment, the first and second helical implants are disposed at respective longitudinal positions with respect to the delivery tool.

In an embodiment, the first and second implants each include a flexible, biocompatible material selected from the group consisting of: nitinol and silicone.

In an embodiment the apparatus includes, a respective needle coupled to at least one end of each of the first and second implants, the needle being configured to puncture tissue of the patient.

In an embodiment, the needle includes a rigid, biocompatible material configured to puncture tissue of the patient.

In an embodiment, the needle includes stainless steel.

In an embodiment, the first and second coiled implants include respective coiled implants that are conically-shaped at least in part.

In an embodiment, a proximal coil near a proximal end of each one of the conically-shaped coiled implants has a diameter that is larger than a diameter of a distal coil near a distal end of each one of the conically-shaped coiled implants.

In an embodiment, the delivery tool is configured to facilitate implantation of the first and second implants around the body lumen by corkscrewing the first and second coiled implants into the tissue.

In an embodiment, the tissue includes a prostate of the patient and the body lumen includes a urethra of the patient, and the delivery tool is configured to facilitate corkscrewing of the first and second coiled implants into the prostate.

In an embodiment, the delivery tool is configured to facilitate the implantation of the first and second implants from within the urethra.

In an embodiment, the delivery tool is configured to facilitate distal advancement of the first and second implants around the urethra by facilitating corkscrewing of the first and second implants.

In an embodiment, the delivery tool is configured to facilitate:

advancement of the first and second implants distally through the body lumen of the patient until the first and second implants emerge at a distal end of the body lumen into a body cavity of the patient, and

implantation of the first and second implants around the body lumen by retracting the first and second implants.

In an embodiment, the delivery tool is configured to facilitate:

rotation of the first implant in a first direction,

rotation of the second implant in a second direction thereof, and

implantation of the first and second implants in a manner in which the second implant is disposed concentrically with respect to the first implant.

In an embodiment, the body lumen includes a urethra of the patient and the body cavity includes a bladder of the patient, and the delivery tool is configured to facilitate the implantation of the first and second implants around the urethra of the patient.

In an embodiment the apparatus includes, first and second sheaths having respective first and second lumens thereof, the first and second lumens configured to helically surround the first and second coiled implants, respectively.

In an embodiment the apparatus includes:

a first ablation tool configured to be slidably advanced through the first lumen, the first sheath is shaped to define at least one first hole at an end of the implant that is configured to puncture tissue of the patient, and a portion of the first ablation tool is configured for advancement through the first hole; and

a second ablation tool configured to be slidably advanced through the second lumen, the second sheath is shaped to define at least one second hole at an end of implant that is configured to puncture tissue of the patient, and a portion of the first ablation tool is configured for advancement through the second hole.

In an embodiment the apparatus includes:

a first flexible tube coupled to a portion of the first coiled implant, the first tube being configured to facilitate passage of a fluid through the lumen of the implant, and the first implant is shaped to define one or more holes configured for passage of the fluid externally to the first implant; and

a second flexible tube coupled to a portion of the second coiled implant, the second tube being configured to facilitate passage of a fluid through the lumen of the implant, and the second implant is shaped to define one or more holes configured for passage of the fluid externally to the second implant.

In an embodiment, the first and second implants include respective first and second hollow, coiled implants shaped to define respective first and second helical lumens thereof.

In an embodiment the apparatus includes:

a first ablation tool configured to be slidably advanced through the first lumen, and the first sheath is shaped to define at least one first hole near an end of the first implant that is configured to puncture tissue of the patient, a portion of the first ablation tool is configured for advancement through the first hole; and

a second ablation tool configured to be slidably advanced through the second lumen, and the second sheath is shaped to define at least one second hole near an end of the second implant that is configured to puncture tissue of the patient, and a portion of the second ablation tool is configured for advancement therethrough the second hole.

In an embodiment the apparatus includes:

a first flexible tube coupled to a portion of the first coiled implant, the first tube being configured to facilitate passage of a fluid through the first lumen of the first implant, and the first implant is shaped to define one or more first holes configured for passage of the fluid externally to the implant; and

a second flexible tube coupled to a portion of the second coiled implant, the second tube being configured to facilitate passage of a fluid through the second lumen of the second implant, and the second implant is shaped to define one or more second holes configured for passage of the fluid externally to the implant.

There is yet additionally provided, in accordance with an embodiment of the present invention, apparatus, including:

a coiled implant including:
a proximal coil, near a proximal end of the coiled implant, the proximal coil having a diameter thereof during a resting state of the coiled implant;
a distal coil, near a distal end of the coiled implant, the distal coil having a diameter thereof during the resting state; and

a plurality of coils disposed between the proximal and distal coils, the coiled implant being shaped in a manner in which, during the resting state thereof, the plurality of coils have respective diameters, the respective diameters of the plurality of coils each being smaller than the diameters of the proximal and distal coils; and

a delivery tool, removably couplable to the coiled implant, the tool configured to facilitate implantation of the implant around a body lumen of a patient.

In an embodiment, the proximal coil is a proximal-most coil of the coiled implant.

In an embodiment, the distal coil is a distal-most coil of the coiled implant.

In an embodiment, the delivery tool is configured to facilitate:

advancement of the implant distally through the body lumen of the patient until the implant emerges at a distal end of the body lumen into a body cavity of the patient, and

subsequent implantation of the implant around the body lumen by retracting the implant.

In an embodiment, the respective diameters of the proximal and distal coils are substantially equal.

In an embodiment, the plurality of coils includes:

a first conically-shaped portion of coils disposed in series in a manner in which one coil thereof is disposed adjacently to the proximal coil, and respective diameters of the coils of the first portion of coils decrease in series from (a) the coil adjacent to the proximal coil to (b) a coil of the coils of the first portion that is furthest from the proximal coil; and

a second conically-shaped portion of coils disposed in series in a manner in which one coil thereof is disposed adjacently to the distal coil, and respective diameters of the coils of the second portion of coils decrease in series from (a) the coil adjacent to the distal coil to (b) a coil of the coils of the second portion that is furthest from the distal coil.

In an embodiment the apparatus includes, a motor coupled to the apparatus, the motor being configured to facilitate implantation of the implant around the body lumen.

In an embodiment, the motor is coupled to the delivery tool.

In an embodiment, the motor is coupled to the implant.

In an embodiment, the motor includes an ultrasound transducer configured to create vibrations in the implant in response to vibrations effected by the ultrasound transducer.

In an embodiment, the motor includes a vibrator configured to create vibrations in the implant in response to vibrations effected by the vibrator.

In an embodiment, the motor is configured to control the implantation of the implant around the body lumen by cycling between:

(a) facilitating advancement, by a first number of degrees, of the implant in a first rotational direction through tissue of the body lumen, and

(b) facilitating retracting, by a second number of degrees, of the implant in a second rotational direction that is opposite the first direction.

There is yet further provided, in accordance with an embodiment of the present invention, apparatus, including:

a plurality of curved needles; and

a delivery tool coupled to the plurality of curved needles, the delivery tool being configured to facilitate advancing of the needles through a lumen of a patient, puncturing by the needles of an inner wall of the lumen, advancing of the needles around the lumen, and decoupling of the needles from the delivery tool.

In an embodiment, each one of the plurality of curved needles is not coupled to one another following the decoupling of the needles from the delivery tool.

In an embodiment, each one of the plurality of curved needles is shaped to define between 180 and 360 degrees in a resting state thereof.

In an embodiment, each one of the plurality of curved needles is shaped to define between 250 and 300 degrees in the resting state thereof.

There is still additionally provided, in accordance with an embodiment of the present invention, apparatus, including:

a plurality of distinct coiled implants; and

a delivery tool configured to simultaneously hold the implants, and facilitate advancement of the implants in a lumen of a patient and implantation of the implants around the lumen.

In an embodiment, the implants are each shaped to define an inner lumen having a diameter that is larger than a diameter of the body lumen.

In an embodiment, the delivery tool is configured to facilitate implantation of the implants around the lumen from within the lumen.

In an embodiment, the delivery tool is configured to facilitate corkscrewing of the implants around the lumen from within the lumen.

In an embodiment, each implant of the plurality of distinct coiled implants is shaped to have 1-5 coils in a resting state thereof.

In an embodiment, the plurality of distinct coiled implants are each shaped to have 3-4 coils in a resting state thereof.

In an embodiment the apparatus includes a motor coupled to the apparatus, the motor being configured to facilitate implantation of the implant around the body lumen.

In an embodiment, the motor is coupled to the delivery tool.

In an embodiment the apparatus includes a plurality of motors, and a respective motor of the plurality of motors is coupled to each implant of the plurality of implants.

In an embodiment, the motor includes an ultrasound transducer configured to create vibrations in the implants in response to vibrations effected by the ultrasound transducer.

In an embodiment, the motor includes a vibrator configured to create vibrations in the implants in response to vibrations effected by the vibrator.

In an embodiment, the motor is configured to control the implantation of the implants around the body lumen by cycling between:

(a) facilitating advancement, by a first number of degrees, of the implants in a first rotational direction through tissue of the body lumen, and

(b) facilitating retracting, by a second number of degrees, of the implants in a second rotational direction that is opposite the first direction.

There is further provided, in accordance with an embodiment of the present invention, apparatus, including:

a conically-shaped coiled implant shaped to define a longitudinal lumen thereof, the implant including at least a proximal coil having an outer surface thereof and a distal coil having an inner surface thereof, the distal coil having a diameter that is larger than the proximal coil;

a plurality of rods configured to be disposed in part within the longitudinal lumen of the implant; and

a delivery tool, removably coupled to the implant, the tool configured to:
facilitate advancement of the implant around a body lumen of a patient in a manner in which the longitudinal lumen of the implant surrounds the body lumen of the patient, and
subsequently, facilitate implantation of the plurality of rods substantially in parallel with and around the body lumen by facilitating advancement of the rods below the inner surface of the distal coil and above the outer surface of the proximal coil.

In an embodiment, the implant includes a flexible, biocompatible material selected from the group consisting of: nitinol and silicone, and the rods include a material selected from the group consisting of: nitinol, silicone, and stainless steel.

In an embodiment, the delivery tool is configured to facilitate implantation of the coiled implant around the body lumen from within the body lumen.

In an embodiment, the delivery tool is configured to facilitate corkscrewing of the coiled implant around the body lumen from within the body lumen.

In an embodiment, the delivery tool is configured to facilitate advancement of the rods into a body cavity at an end of the lumen, and implantation of the rods around the body lumen from within the body cavity.

In an embodiment, the delivery tool is configured to facilitate corkscrewing of the coiled implant around the body lumen from within the body cavity.

In an embodiment, the body cavity includes a bladder of the patient and the body lumen includes a urethra of the patient, and the delivery tool is configured to facilitate corkscrewing of the coiled implant around the body lumen from within the body cavity.

There is still further provided, in accordance with an embodiment of the present invention, apparatus, including:

an elongate coiled structure having a lumen and a transverse cross-sectional shape selected from the group consisting of: a square, a diamond, and a triangle, the elongate structure being resorbable by a body lumen of a patient; and

a delivery tool reversibly couplable to the elongate structure and configured to facilitate advancement of the structure to a vicinity within the body lumen of the patient.

In an embodiment, the elongate structure includes a pro-fibrotic coating.

In an embodiment, the body lumen includes a urethra of the patient and the vicinity within the body lumen includes a portion of the urethra that is surrounded by a prostate of the patient, and the elongate structure is configured to be disposed in the portion of the urethra that is surrounded by the prostate of the patient.

In an embodiment, the elongate structure includes a radially-expandable structure configured to expand in the vicinity of the body lumen.

In an embodiment, the elongate structure includes a plurality of successively-disposed coils defining respective areas between the successive coils, and the coils are configured to pinch tissue into the areas between the successive coils.

In an embodiment, the elongate structure includes a wire defining the elongate structure, and the wire shaped to define a shape in cross-section thereof, the shape being selected from the group consisting of: a triangle, a square, a diamond, a circle, and an ellipse.

In an embodiment the apparatus includes, a mechanical element selected from the group consisting of: a stent, a balloon, a wire and basket, and the mechanical element is disposed within the lumen of the elongate structure between the elongate structure and the delivery tool, and the mechanical structure is configured to radially expand the elongate structure.

In an embodiment, the elongate structure includes stainless steel.

There is additionally provided, in accordance with an embodiment of the present invention, a method, including:

advancing through a body lumen of a patient first and second coiled implants, an outer diameter of the second implant being smaller than an inner diameter of the first implant, one of the coiled implants being right-handed and one of the coiled implants being left-handed; and

implanting the first and second implants around the body lumen in a manner in which, subsequently to the implanting, the second implant is disposed concentrically with respect to the first implant.

In an embodiment, implanting the first and second implants includes:

rotating the first implant in a first direction; and

rotating the second implant in a second direction that is opposite the first direction.

In an embodiment, implanting the first and second implants includes implanting the first and second implants simultaneously.

In an embodiment, implanting the first and second implants includes implanting the first and second implants in sequence.

In an embodiment, implanting the first and second implants includes cycling between:

(a) advancing, by a first number of degrees, the first and second implants in their respective first rotational directions through tissue of the body lumen, and

(b) retracting, by a second number of degrees, the first and second implants in a second rotational direction that is opposite the first direction.

In an embodiment the method includes, advancing the first and second implants through the body lumen and into a body cavity of the patient prior to the implanting, and implanting the first and second implants around the lumen includes implanting the first and second implants around the body lumen by proximally corkscrewing the implants around the body lumen from within the body cavity.

In an embodiment:

the body lumen includes a urethra of the patient and the body cavity includes a bladder of the patient,

advancing the implants through the body lumen and into the body cavity of the patient includes advancing the implants through the urethra and into the bladder of the patient, and

proximally corkscrewing the implants around the body lumen from within the body cavity includes proximally corkscrewing the implants around the urethra from within the bladder.

In an embodiment, implanting the first and second implants around the body lumen of the patient includes implanting the implants around the body lumen of the patient from within the body lumen.

In an embodiment, implanting the first and second implants around the body lumen of the patient from within the body lumen includes corkscrewing the first and second implants around the lumen from within the lumen.

In an embodiment the method includes, vibrating the first and second implants during the implanting.

In an embodiment, vibrating the first and second implants includes mechanically vibrating the implants.

In an embodiment, vibrating the first and second implants includes vibrating the implants by applying to the implants ultrasound energy.

There is yet additionally provided, in accordance with an embodiment of the present invention, a method, including:

advancing through a body lumen of a patient, a coiled implant including:
a proximal coil, near a proximal end of the coiled implant, the proximal coil having a diameter thereof during a resting state of the coiled implant,
a distal coil, near a distal end of the coiled implant, the distal coil having a second diameter thereof during the resting state, and

a plurality of coils disposed between the proximal and distal coils, the coiled implant being shaped in a manner in which, during the resting state thereof, the plurality of coils have respective diameters, the respective diameters of the plurality of coils each being smaller than the diameters of the proximal and distal coils; and

implanting the implant around the body lumen of the patient.

In an embodiment, implanting the implant includes cycling between:

(a) advancing, by a first number of degrees, the implant in a first rotational direction through tissue of the body lumen, and

(b) retracting, by a second number of degrees, the implant in a second rotational direction that is opposite the first direction.

In an embodiment the method includes, advancing the implant through the body lumen and into a body cavity of the patient prior to the implanting, and implanting the implant around the lumen includes implanting the implant around the body lumen by proximally corkscrewing the implant around the body lumen from within the body cavity.

In an embodiment:

the body lumen includes a urethra of the patient and the body cavity includes a bladder of the patient,

advancing the implant through the body lumen and into the body cavity of the patient includes advancing the implant through the urethra and into the bladder of the patient, and

proximally corkscrewing the implant around the body lumen from within the body cavity includes proximally corkscrewing the implant around the urethra from within the bladder.

In an embodiment, implanting the implant around the body lumen of the patient includes implanting the implant around the body lumen of the patient from within the body lumen.

In an embodiment, implanting the implant around the body lumen of the patient from within the body lumen includes corkscrewing the implant around the body lumen from within the lumen.

In an embodiment the method includes, vibrating the implant during the implanting.

In an embodiment, vibrating the implant includes mechanically vibrating the implant.

In an embodiment, vibrating the implant includes vibrating the implant by applying ultrasound energy to the implant.

There is further provided, in accordance with an embodiment of the present invention, a method including:

advancing a plurality of curved needles through a body lumen of a patient; and maintaining an open state of the body lumen by implanting the plurality of curved needles around at least a part of the body lumen of the patient.

In an embodiment, implanting the plurality of curved needles around the body lumen includes implanting the plurality of curved needles around the body lumen from within the urethra.

In an embodiment, implanting the plurality of curved needles around the body lumen includes puncturing an inner wall of the body lumen by the plurality of curved needles.

There is additionally provided, in accordance with an embodiment of the present invention, a method, including:

simultaneously advancing a plurality of distinct coiled implants through a body lumen of a patient; and

implanting the plurality of implants around the lumen.

In an embodiment, implanting the plurality of implants includes cycling between:

(a) advancing, by a first number of degrees, the implants in a first rotational direction through tissue of the body lumen, and

(b) retracting, by a second number of degrees, the implants in a second rotational direction that is opposite the first direction.

In an embodiment the method includes, vibrating the implants during the implanting.

In an embodiment, vibrating the implants includes mechanically vibrating the implants.

In an embodiment, vibrating the implants includes vibrating the implants by applying ultrasound energy to the implants.

In an embodiment, implanting the first and second implants around the body lumen of the patient includes implanting the implants around the body lumen of the patient from within the body lumen.

In an embodiment, implanting the first and second implants around the body lumen of the patient from within the body lumen includes corkscrewing the first and second implants around the lumen from within the lumen.

There is further provided, in accordance with an embodiment of the present invention, a method, including:

advancing, through a body lumen of a patient, a conically-shaped coiled implant shaped to define a longitudinal lumen thereof, the implant including at least a proximal coil having an outer surface thereof and a distal coil having an inner surface thereof, the distal coil having a diameter that is larger than the proximal coil;

implanting the implant around the body lumen of the patient;

advancing a plurality of rods toward the implant; and

implanting the rods in part within the longitudinal lumen of the implant and substantially in parallel with and around the body lumen by advancing the plurality of rods below the inner surface of the distal coil and above the outer surface of the proximal coil.

There is yet further provided, in accordance with an embodiment of the present invention, a method, including:

advancing through a body lumen of a patient an elongate coiled structure having a lumen and a transverse cross-sectional shape selected from the group consisting of: a square, a diamond, and a triangle, the elongate structure being resorbable by a body lumen of a patient; and

releasing the elongate structure in a vicinity within the body lumen of the patient.

In an embodiment, releasing the elongate structure includes facilitating expansion of the elongate structure within the body lumen.

There is yet additionally provided, in accordance with an embodiment of the present invention, a method, including:

advancing an implant to a vicinity of soft tissue of a body lumen of the patient;

implanting the implant in the soft tissue by applying a jackhammer force to the implant during the implanting.

In an embodiment, applying the jackhammer force to the implant includes remotely applying the force to the implant.

There is still further provided, in accordance with an embodiment of the present invention, apparatus, including:

a transurethral delivery tool insertable in a urethra of a patient, the tool having a flexible distal tip that is:
deflectable from a position that is aligned with a longitudinal axis of the tool, and
when deflected, operative to compress tissue of a prostate of the patient, by pushing a wall of the urethra; and

at least one implant that is deliverable to a portion of the compressed tissue and configured to maintain the tissue in a compressed state upon withdrawal of the delivery tool from the urethra.

In an embodiment the apparatus includes, an imaging device couplable to the delivery tool.

In an embodiment, the at least one implant includes a plurality of implants, and the delivery tool is configured to implant the plurality of implants by orienting the implants radially with respect to a portion of the urethra.

In an embodiment, the at least one implant includes a plurality of implants, and the delivery tool is configured to implant each of the plurality of implants at respective transverse planes of the urethra that are disposed along a longitudinal axis of the urethra.

In an embodiment, the delivery tool is configured to implant the implant in a manner in which the implant is fully embedded within the tissue and does not extend within the urethra.

In an embodiment, the implant includes a coiled implant defining an implant lumen having a longitudinal axis thereof, and the delivery tool is configured to orient the implant with respect to the urethra of the patient in a manner in which:

the implant is disposed entirely within the tissue of the prostate of the patient, and

the longitudinal axis of the implant lumen defined by the implant is disposed at a nonzero angle with respect to a longitudinal axis of the urethra.

In an embodiment, the delivery tool is configured to deliver the implant in a manner in which the longitudinal axis of the implant lumen is disposed substantially perpendicularly with respect to the longitudinal axis of the urethra.

In an embodiment the implant includes:

a body portion including a plurality of successive contiguous coils and defining a longitudinal axis of the implant,

a first end including a first coil that is at a first end of the body portion, the first end being configured to puncture urethral tissue of the patient; and

a second end including a second coil that is at a second end of the body portion, the second end being configured to be disposed within the prostate tissue.

In an embodiment the apparatus includes, a wire, and the at least one implant includes a plurality of implants, and the plurality of implants are coupled to each other by means of the wire.

In an embodiment:

the wire has first and second ends and a portion disposed between the first and second ends, the portion having a first-end-to-second-end length,

the wire has a longitudinal axis measured along the length, and

each of the implants is coupled to the wire at successive sites along the longitudinal axis of the wire.

In an embodiment, the delivery tool is configured to implant the plurality of implants such that upon implantation, each of the implants is coupled to the wire at successive sites along the longitudinal axis of the wire.

In an embodiment, the implant is longitudinally compressible following implantation and configured to further compress the prostate tissue.

In an embodiment, the implant is longitudinally compressible following implantation, in response to an application of energy thereto by an energy source.

In an embodiment, the implant is longitudinally compressible, in response to an increase in temperature of the implant as a result of implantation.

In an embodiment, the implant includes a screw implant defining an implant body having a longitudinal axis thereof, and the delivery tool is configured to orient the implant with respect to the urethra of the patient in a manner in which:

the implant is at least partially disposed within the tissue of the prostate of the patient, and

the longitudinal axis of the implant body is disposed at a nonzero angle with respect to a longitudinal axis of the urethra.

In an embodiment, the delivery tool is configured to deliver the implant in a manner in which the longitudinal axis of the implant body is disposed substantially perpendicularly with respect to the longitudinal axis of the urethra.

In an embodiment, the delivery tool is configured to implant the implant in a manner in which the implant is partially embedded within the prostate tissue in a manner in which the implant (a) does not extend beyond a prostate capsule of the patient, and (b) is partially disposed within the urethra.

In an embodiment, the implant includes a screw implant including: a body portion having a longitudinal axis thereof,

a first end including a head portion that is at a first end of the body portion; and

a second end including a pointed tip portion that is at a second end of the body portion, the pointed tip being configured to puncture urethral tissue of the patient.

In an embodiment, at least a portion of the implant includes a biodegradable material.

In an embodiment, the portion of the implant including the biodegradable material further includes a medication.

In an embodiment, the medication includes a medication for treatment of benign prostatic hyperplasia.

In an embodiment, the implant includes at least one flexible curved implant shaped to define an arc of up to 360 degrees in an expanded state thereof.

In an embodiment, the flexible curved implant, in the expanded state, is shaped to define a plane having a normal thereto, and the delivery tool is configured to implant the implant in a manner in which the normal to the plane defined by the implant is substantially parallel to a longitudinal axis of the urethra.

In an embodiment, the flexible curved implant includes a resilient curved implant, the resilient curved implant:

in an expanded state thereof, is shaped to define an arc of up to 360 degrees and a plane having a normal thereto,

is implantable in prostate tissue surrounding the urethra of a patient and configured to move the prostate tissue away from a longitudinal axis of the urethra,

during implantation, has a first configuration thereof in which the implant defines a first radius of curvature,

following implantation, assumes a second configuration thereof in which the implant defines a second radius of curvature, and

while transitioning between the first and second configurations, is operative to radially push the prostate tissue.

In an embodiment, the first radius of curvature is smaller than the second radius of curvature.

In an embodiment, the at least one flexible curved implant includes a plurality of flexible curved implants, and the delivery tool is operative to implant the plurality of flexible curved implants around a portion of the urethra.

In an embodiment, the delivery tool is operative to implant:

a first one of the plurality of implants at least in part in a first lobe of the prostate of the patient, and

a second one of the plurality of implants at least in part in a second lobe of the prostate of the patient.

In an embodiment, the delivery tool is operative to implant:

the first one of the plurality of implants entirely within the first lobe, and

the second one of the plurality of implants entirely within the second lobe.

In an embodiment the apparatus includes, at least one inflatable element coupled to the delivery tool at a distal portion thereof, the inflatable element is configured to be inflated in a manner in which the inflatable element:

contacts an inner wall of the urethra and applies pressure thereto, and

stabilizes the delivery tool during deflection of the distal tip and implantation of the implant.

In an embodiment, the inflatable element has a volume in an inflated state thereof that is 1-50 cc.

In an embodiment, the inflatable element includes an annular inflatable element surrounding the distal portion of the delivery tool.

In an embodiment, the inflatable element is further operative to compress the prostate tissue and maintain the prostate tissue in a compressed state during implantation of the implant.

There is still additionally provided, in accordance with an embodiment of the present invention, apparatus, including:

a transurethral delivery tool insertable in a urethra of a patient, the tool being configured to compress tissue of a prostate by pushing a wall of the urethra;

at least first and second implants deliverable to a portion of the compressed tissue and configured to maintain the tissue in a compressed state upon withdrawal of the delivery tool from the urethra; and

a flexible longitudinal member coupled at a first portion thereof to the first implant and at a second portion thereof to the second implant, the longitudinal member having an extendable portion between the first and second portions thereof,

the delivery tool is configured to:
implant the first implant at a first location in a first portion of the tissue of the prostate, and

extend the extendable portion of the wire to a second location in tissue surrounding the urethra by implanting the second implant at the second location in a second portion of the tissue of the prostate.

In an embodiment, the at least first and second implants are configured to compress the respective first and second portion of the tissue of the prostate, and the extendable portion is configured to provide supplemental radial compressing of the tissue of the prostate.

In an embodiment, each one of the first and second implants includes:

a body portion including a plurality of successive contiguous coils, the body portion defining a longitudinal axis of the implant;

a first end including a first coil that is at a first end of the body portion, the first end being configured to puncture urethral tissue of the patient; and

a second end including a second coil that is at a second end of the body portion, the second end being configured to be disposed within the prostate tissue.

In an embodiment, the at least first and second implants includes a plurality of implants, and the plurality of implants are coupled to each other by being coupled to the wire at respective locations along the wire.

In an embodiment:

the wire has first and second ends and a portion disposed between the first and second ends, the portion having a first-end-to-second-end length,

the wire has a longitudinal axis measured along the length of the portion, and

each of the plurality of implants is coupled to the wire at successive sites along the longitudinal axis of the wire.

In an embodiment, the delivery tool is configured to implant the plurality of implants such that upon implantation, each of the implants is coupled to successive sites along the longitudinal axis of the wire.

In an embodiment, each of the at least first and second implants is longitudinally compressible along the longitudinal axis thereof following implantation to further compress the tissue of the prostate.

There is yet additionally provided, in accordance with an embodiment of the present invention, apparatus, including:

a transurethral delivery tool insertable in a urethra of a patient, and configured to compress tissue of a prostate by pushing a wall of the urethra; and

at least one rod implantable in tissue of the prostate; and

at least one implant that is deliverable by the delivery tool to a portion of the compressed tissue, at least a portion of the implant being couplable to the rod to maintain the tissue in a compressed state upon withdrawal of the delivery tool from the urethra.

In an embodiment, the delivery tool includes a flexible distal tip that is deflectable from a position that is aligned with a longitudinal axis of the tool, and when deflected, operative to additionally compress tissue of the prostate of the patient, by pushing the wall of the urethra.

In an embodiment:

the rod has first and second ends and a portion disposed between the first and second ends, the portion having a first-end-to-second-end length,

the rod has a longitudinal axis measured along the length of the portion,

the at least one implant includes a plurality of implants, and

each of the plurality of implants is coupled at at least respective portions thereof to the rod at successive sites along the longitudinal axis of the rod.

In an embodiment:

the rod has first and second ends and a portion disposed between the first and second ends, the portion having a first-end-to-second-end length,

the rod has a longitudinal axis measured along the length of the portion,

the at least one implant includes a plurality of implants, and

the delivery tool is configured to implant the plurality of implants such that upon implantation, each of the plurality of implants is coupled at at least a portion thereof to successive sites along the longitudinal axis of the rod.

In an embodiment, the at least one implant is longitudinally compressible following implantation and configured to further compress the prostate tissue.

In an embodiment, the at least one rod includes a plurality of rods.

There is yet further provided, in accordance with an embodiment of the present invention, apparatus, including:

at least one implant; and

a transurethral delivery tool insertable in a urethra of a patient, configured to deliver the implant in a manner in which:
the implant is disposed entirely within a portion of tissue of a prostate of the patient, and
a longitudinal axis of a lumen of the implant is disposed at a nonzero angle with respect to a longitudinal axis of the urethra.

In an embodiment, the implant includes:

a body portion including a plurality of successive contiguous coils and defining an implant lumen having a longitudinal axis thereof;

a first end including a first coil that is at a first end of the body portion, the first end being configured to puncture urethral tissue of a patient; and

a second end including a second coil that is at a second end of the body portion, the second end being configured to be disposed within tissue of a prostate of the patient.

In an embodiment, the delivery tool is configured to implant the implant in a manner in which the implant is fully embedded within the prostate tissue and does not extend within the urethra.

In an embodiment, the delivery tool is configured to implant the implant in a manner in which the implant is fully embedded within the prostate tissue and does not extend beyond a prostate capsule of the prostate of the patient.

In an embodiment, the delivery tool is configured to deliver the implant in a manner in which the longitudinal axis of the implant lumen is disposed substantially perpendicularly with respect to the longitudinal axis of the urethra.

In an embodiment, the at least one implant includes a plurality of implants, and the delivery tool is configured to implant the plurality of implants by orienting the implants radially with respect to a portion of the urethra.

In an embodiment, the at least one implant includes a plurality of implants, and the delivery tool is configured to implant each of the plurality of implants at respective transverse planes of the urethra that are disposed along the longitudinal axis of the urethra.

In an embodiment:

the delivery tool:
when deflected, is operative to compress the prostate tissue by pushing a wall of the urethra, and
operative to implant the implant in a portion of the compressed prostate tissue, and

the implant is configured to maintain the portion of the prostate tissue in a compressed state upon withdrawal of the delivery tool from the urethra.

In an embodiment, the implant following implantation is longitudinally compressible along the longitudinal axis of the lumen and configured to compress the portion of tissue of the prostate.

In an embodiment, the implant is longitudinally compressible following implantation in response to an application of energy thereto by an energy source disposed externally to the body of the patient and not in contact with the implant.

In an embodiment, the implant is longitudinally compressible, in response to an increase in temperature of the implant as a result of implantation.

In an embodiment the apparatus includes, at least one inflatable element coupled to the delivery tool at a distal portion thereof, and the inflatable element is configured to be inflated in a manner in which the inflatable element:

contacts an inner wall of the urethra and applies pressure thereto, and

stabilizes the delivery tool during implantation of the implant.

In an embodiment, the inflatable element is operative to compress the portion of the prostate tissue and maintain the prostate tissue in a compressed state during implantation of the implant.

There is still further provided, in accordance with an embodiment of the present invention, apparatus, including:

at least one flexible curved implant shaped to define an arc of up to 360 degrees in an expanded state thereof, the implant being implantable in prostate tissue surrounding a urethra; and

a transurethral delivery tool having a delivery tool lumen thereof for housing the implant in a compressed state thereof, and shaped to define an opening in a surface of the delivery tool, through which the implant passes, changing from the compressed state to the expanded state as a result of passing through the opening,

the implant in the expanded state being shaped to define a plane having a normal thereto, and the delivery tool being configured to implant the implant in a manner in which the normal to the plane defined by the implant is substantially parallel to a longitudinal axis of the urethra.

In an embodiment, the delivery tool is configured to implant the implant in a manner in which the implant is fully embedded within the prostate tissue and does not extend beyond a prostate capsule of a prostate of the patient.

In an embodiment, while transitioning between the compressed state and the expanded state, the implant is operative to radially push the prostate tissue.

In an embodiment the apparatus includes, a curved implantation-facilitating sleeve configured to surround the implant as the implant is housed in the delivery tool lumen, the implantation-facilitating sleeve being:

disposed in a compressed state thereof while disposed within the delivery tool lumen,

while surrounding the implant, advanceable though the opening in the surface of the delivery tool,

changeable from the compressed state thereof to an expanded state thereof as a result of passing through the opening, and

shaped to define a pointed tip configured to puncture the prostate tissue and create a channel therein for passage of the implant.

In an embodiment, following implantation of the implant in the channel of the prostate tissue, the sleeve is retractable back into the delivery tool lumen.

In an embodiment, while transitioning between the compressed state and the expanded state, the sleeve is operative to radially push the prostate tissue.

In an embodiment, the flexible curved implant includes a resilient curved implant:

the resilient curved implant, in an expanded state thereof, is shaped to define an arc of up to 360 degrees and a plane having a normal thereto, the implant being implantable in prostate tissue surrounding the urethra of a patient and configured to move the prostate tissue away from a longitudinal axis of the urethra,

during implantation, having a first configuration thereof in which the implant defines a first radius of curvature,

following implantation, assuming a second configuration thereof in which the implant defines a second radius of curvature, and

while transitioning between the first and second configurations, radially pushing the prostate tissue.

In an embodiment, the first radius of curvature is smaller than the second radius of curvature.

In an embodiment, at least one inflatable element coupled to the delivery tool at a distal portion thereof, the inflatable element is configured to be inflated in a manner in which the inflatable element:

contacts an inner wall of the urethra and applies pressure thereto, and

stabilizes the delivery tool during implantation of the implant.

In an embodiment, the inflatable element has a volume in an inflated state thereof that is 1-50 cc.

In an embodiment, the inflatable element includes an annular inflatable element surrounding the distal portion of the delivery tool.

In an embodiment, the inflatable element is operative to compress the portion of the prostate tissue and maintain the tissue in a compressed state during implantation of the implant.

In an embodiment, the inflatable element has a volume in an inflated state thereof that is 1-50 cc.

In an embodiment, the inflatable element includes an annular inflatable element surrounding the distal portion of the delivery tool.

In an embodiment, the at least one flexible curved implant includes a plurality of flexible curved implants, and the delivery tool is operative to implant the plurality of flexible curved implants by placing the implants around a portion of the urethra.

In an embodiment, the delivery tool is operative to implant:

a first one of the plurality of implants at least in part in a first lobe of a prostate of the patient, and

a second one of the plurality of implants at least in part in a second lobe of the prostate of the patient.

In an embodiment, the delivery tool is operative to implant:

the first one of the plurality of implants entirely within the first lobe, and

the second one of the plurality of implants entirely within the second lobe.

There is further provided, in accordance with an embodiment of the present invention, apparatus including:

at least one resilient curved implant, in an expanded state thereof, being shaped to define an arc of up to 360 degrees and a plane having a normal thereto, the implant being implantable in prostate tissue surrounding a urethra of a patient and configured to move the prostate tissue away from a longitudinal axis of the urethra, the implant:
during implantation, having a first configuration thereof in which the implant defines a first radius of curvature,
following implantation, assuming a second configuration thereof in which the implant defines a second radius of curvature, and
while transitioning between the first and second configurations, radially pushing the prostate tissue; and

a transurethral delivery tool being configured to implant the implant in a manner in which the normal to the plane defined by the implant is substantially parallel to a longitudinal axis of the urethra.

In an embodiment, the first radius of curvature is smaller than the second radius of curvature.

In an embodiment, the delivery tool is configured to implant the implant in a manner in which the implant is fully embedded within the prostate tissue and does not extend within the urethra.

In an embodiment, the delivery tool is configured to implant the implant in a manner in which the implant is fully embedded within the prostate tissue and does not extend beyond a prostate capsule of a prostate of the patient.

In an embodiment the apparatus includes, at least one inflatable element coupled to the delivery tool at a distal portion thereof, the inflatable element is configured to be inflated in a manner in which the inflatable element:

contacts an inner wall of the urethra and applies pressure thereto, and

stabilizes the delivery tool during implantation of the implant.

In an embodiment, the inflatable element has a volume in an inflated state thereof that is 1-50 cc.

In an embodiment, the inflatable element is operative to compress the prostate tissue and maintain the tissue in a compressed state during implantation of the implant.

In an embodiment, the inflatable element includes an annular inflatable element surrounding the distal portion of the delivery tool.

In an embodiment the apparatus includes, a curved implantation-facilitating sleeve configured to surround the implant as the implant is housed in the delivery tool lumen, the implantation-facilitating sleeve being:

disposed in a compressed state thereof while disposed within the delivery tool lumen,

while surrounding the implant, advanceable though the opening in the surface of the delivery tool,

changeable from the compressed state thereof to an expanded state thereof as a result of passing through the opening, and

shaped to define a pointed tip configured to puncture the prostate tissue and create a channel therein for passage of the implant.

In an embodiment, following implantation of the implant in the channel of the prostate tissue, the sleeve is retractable back into the delivery tool lumen.

In an embodiment, while transitioning between the compressed state and the expanded state, the sleeve is operative to radially push the prostate tissue.

In an embodiment, the at least one flexible curved implant includes a plurality of flexible curved implants, and the delivery tool is operative to implant the plurality of flexible curved implants by placing the implants around a portion of the urethra.

In an embodiment, the delivery tool is operative to implant:

a first one of the plurality of implants at least in part in a first lobe of a prostate of the patient, and

a second one of the plurality of implants at least in part in a second lobe of the prostate of the patient.

In an embodiment, the delivery tool is operative to implant:

the first one of the plurality of implants entirely within the first lobe, and

the second one of the plurality of implants entirely within the second lobe.

There is additionally provided, in accordance with an embodiment of the present invention, a method, including:

transurethrally advancing a flexible, distal tip of a delivery tool through a urethra of a patient;

deflecting the distal tip of the tool from a position that is aligned with a longitudinal axis of the tool, and, by the deflecting of the distal tip, compressing tissue of a prostate of the patient by pushing a wall of the urethra; and

maintaining the tissue in a compressed state by implanting at least one implant in the compressed prostate tissue.

In an embodiment, maintaining the tissue in the compressed state includes maintaining the tissue in the compressed state following removal of the delivery tool from the urethra.

In an embodiment, implanting the implant includes implanting the implant in the prostate tissue in a manner in which the implant is fully embedded within the prostate tissue and does not extend beyond a prostate capsule of the patient.

In an embodiment, implanting the implant includes implanting the implant in the prostate tissue in a manner in which the implant is partially embedded within the prostate tissue.

In an embodiment, implanting the implant includes implanting the implant in the compressed tissue in a manner in which the implant is fully embedded within the tissue and does not extend within the urethra.

In an embodiment, implanting the at least one implant includes implanting a plurality of implants in the prostate tissue, and implanting the plurality of implants includes implanting the plurality of implants orienting the implants radially with respect to a portion of the urethra.

In an embodiment, implanting the at least one implant includes implanting a plurality of implants in the prostate tissue, and implanting the plurality of implants includes implanting the plurality of implants at respective transverse planes of the urethra that are disposed along a longitudinal axis of the urethra.

In an embodiment the method includes, adjusting a configuration of the implant following implantation thereof by applying energy to the implant from an energy source disposed externally to a body of the patient and not in contact with the implant.

In an embodiment the method includes, stabilizing the delivery tool during the deflecting by inflating at least one inflatable element to 1-50 cc in a manner in which the inflatable element contacts an inner wall of the urethra.

In an embodiment, implanting the implant includes implanting a longitudinally-compressible implant and facilitating further compressing of the prostate tissue in response to longitudinal compressing of the longitudinally-compressible implant.

In an embodiment, implanting the implant includes implanting the implant at a non-zero angle with respect to a longitudinal axis of the urethra.

In an embodiment, implanting the implant at the non-zero angle includes implanting the implant substantially perpendicularly with respect to the longitudinal axis of the urethra.

In an embodiment, implanting the implant includes:

implanting in the prostate tissue at least one flexible curved implant shaped to define an arc of up to 360 degrees in an expanded state thereof, and

further compressing the prostate tissue in response to the implanting.

In an embodiment, implanting the flexible curved implant includes implanting the implant in a manner in which a normal to a plane defined by the implant is substantially parallel to the longitudinal axis of the urethra.

In an embodiment, implanting the at least one flexible curved implant includes implanting a plurality of flexible curved implants around a portion of the urethra.

In an embodiment, implanting the plurality of implants includes:

implanting a first one of the plurality of implants at least in part in a first lobe of the prostate of the patient, and

implanting a second one of the plurality of implants at least in part in a second lobe of the prostate of the patient.

In an embodiment:

implanting the first one of the plurality of implants at least in part in the first lobe of the prostate of the patient includes implanting the first one of the plurality of implants entirely within the first lobe, and

implanting the second one of the plurality of implants at least in part in the second lobe of the prostate of the patient includes implanting the second one of the plurality of implants entirely within the second lobe.

In an embodiment, implanting the flexible curved implant includes:

implanting a resilient curved implant having a first configuration thereof in which the implant defines a first radius of curvature during the implanting and, following the implanting, a second configuration thereof in which the implant defines a second radius of curvature, and

radially pushing the prostate tissue by the implant transitioning between the first and second configurations.

There is yet additionally provided, in accordance with an embodiment of the present invention, a method, including:

transurethrally advancing through a urethra of a patient at least one flexible curved implant shaped to define an arc of up to 360 degrees in an expanded state thereof;

compressing tissue of a prostate of the patient by implanting the implant in the tissue of the prostate in a manner in which:
a normal to a plane defined by the implant is substantially parallel to a longitudinal axis of the urethra, and
the implant pushes the tissue of the prostate away from a longitudinal axis of the urethra.

In an embodiment, implanting the implant includes implanting the implant in the tissue of the prostate in a manner in which the implant is fully embedded within the tissue of the prostate and does not extend beyond a prostate capsule of the patient.

In an embodiment, implanting the at least one implant includes implanting a plurality of implants in the prostate tissue, and implanting the plurality of implants includes implanting the plurality of implants at respective transverse planes of the urethra that are disposed along a longitudinal axis of the urethra.

In an embodiment the method includes, adjusting a configuration of the implant following implantation thereof by applying energy to the implant from an energy source.

In an embodiment, implanting the flexible curved implant includes:

implanting a resilient curved implant having a first configuration thereof in which the implant defines a first radius of curvature during the implanting and, following the implanting, a second configuration thereof in which the implant defines a second radius of curvature, and

radially pushing the tissue of the prostate by the implant transitioning between the first and second configurations.

In an embodiment, the method further includes compressing the prostate tissue by inflating an inflatable element to 1-50 cc.

In an embodiment, implanting the implant includes implanting the implant such that the implant maintains the tissue in a compressed state thereof following the compressing.

In an embodiment, transurethrally advancing the implant includes transurethrally advancing the implant in a compressed state thereof in a lumen of a delivery tool, and implanting the implant includes:

advancing the implant through an opening of the delivery tool and into the tissue of the prostate, and

implanting the implant such that the implant maintains the tissue in a pushed state thereof following removal of the delivery tool from the urethra.

In an embodiment, advancing the implant through the opening includes advancing the implant surrounded by a sleeve, and creating a channel in the tissue of the prostate by the sleeve.

In an embodiment the method includes, stabilizing the delivery tool during the implanting by inflating at least one inflatable element to 1-50 cc in a manner in which the inflatable element contacts an inner wall of the urethra.

In an embodiment, implanting the at least one implant includes implanting a plurality of implants in the prostate tissue, and implanting the plurality of implants includes implanting the plurality of implants by orienting the implants radially with respect to a portion of the urethra.

In an embodiment, implanting the plurality of implants includes:

implanting a first one of the plurality of implants at least in part in a first lobe of the prostate of the patient; and

implanting a second one of the plurality of implants at least in part in a second lobe of the prostate of the patient.

In an embodiment:

implanting the first one of the plurality of implants at least in part in the first lobe of the prostate of the patient includes implanting the first one of the plurality of implants entirely within the first lobe, and

implanting the second one of the plurality of implants at least in part in the second lobe of the prostate of the patient includes implanting the second one of the plurality of implants entirely within the second lobe.

There is further provided, in accordance with an embodiment of the present invention, a method, including:

transurethrally advancing through a urethra of a patient at least one coiled implant including:

a plurality of successive contiguous coils and defining a lumen having a longitudinal axis in an expanded state thereof; and

compressing tissue of a prostate of the patient by implanting the implant in the tissue of the prostate in a manner in which the implant moves the tissue of the prostate away from a longitudinal axis of the urethra by the implant changing from an expanded to a compressed state.

In an embodiment, implanting the implant includes implanting the implant in the tissue of the prostate in a manner in which the implant is fully embedded within the tissue of the prostate and does not extend beyond a prostate capsule of the patient.

In an embodiment, implanting the at least one implant includes implanting a plurality of implants in the prostate tissue, and implanting the plurality of implants includes implanting the plurality of implants at respective transverse planes of the urethra that are disposed along a longitudinal axis of the urethra.

In an embodiment the method includes, adjusting a configuration of the implant following implantation thereof by applying energy to the implant from an energy source.

In an embodiment the method includes, adjusting a configuration of the implant by increasing a temperature of the implant as a result of the implanting.

In an embodiment, implanting the coiled implant includes:

implanting a coiled implant having a first configuration thereof in which the implant defines a larger, expanded configuration during the implanting and, following the implanting, a second configuration thereof in which the implant defines a smaller, compressed configuration; and

radially moving the tissue of the prostate by the implant transitioning between the first and second configurations.

There is yet further provided, in accordance with an embodiment of the present invention, a method including:

transurethrally advancing a flexible, distal tip of a delivery tool through a urethra of a patient;

deflecting the distal tip of the tool from a position that is aligned with a longitudinal axis of the tool, and, by the deflecting of the distal tip, compressing tissue of a prostate of the patient by pushing a wall of the urethra; and

treating the prostate tissue with medication by implanting an at least partially biodegradable implant including the medication, the implant maintaining the compression of the prostate.

The present invention will be more fully understood from the following detailed description of embodiments thereof, taken together with the drawings, in which:

### BRIEF DESCRIPTION OF THE DRAWINGS

FIG. 1 is a schematic illustration of a delivery tool being introduced within a constricted urethra of a patient, in accordance with an embodiment of the present invention;

FIG. 2 is a schematic illustration of an implant disposed in a compressed state at a distal end of the delivery tool of FIG. 1, in accordance with an embodiment of the present invention;

FIGS. 3 and 4 are schematic illustrations of the implant of FIG. 2 expanding once inside a bladder of the patient, in accordance with an embodiment of the present invention;

FIG. 5 is a schematic illustration of the implant of FIG. 2 being implanted around the urethra in a prostate of the patient, in accordance with an embodiment of the present invention;

FIG. 6 is a schematic illustration of the implant of FIG. 2 implanted within the prostate of the patient, in accordance with an embodiment of the present invention;

FIG. 7 is a schematic illustration of an extraction tool being advanced into the bladder of the patient, in accordance with an embodiment of the present invention;

FIG. 8 is a schematic illustration of the extraction tool removing the implant from the prostate of the patient, in accordance with an embodiment of the present invention;

FIG. 9 is a schematic illustration of the implant being extracted from the body of the patient, in accordance with an embodiment of the present invention;

FIG. 10 is a schematic illustration of the delivery tool coupled to first and second coiled implants, in accordance with an embodiment of the present invention;

FIG. 11 is a schematic illustration of the delivery tool coupled to a conic coiled implant, in accordance with an embodiment of the present invention;

FIG. 12 is a schematic illustration of an implant configured to be implanted around a body lumen of the patient, in accordance with an embodiment of the present invention;

FIG. 13A is a schematic illustration of the delivery tool and an implant coupled thereto, in accordance with another embodiment of the present invention;

FIGS. 13B-C are schematic illustrations of a cross-section of a wire shaped to define the implant of FIG. 13A, in accordance with respective embodiments of the present invention;

FIG. 13D is a schematic illustration of the implant of FIG. 13A implanted around the urethra of the patient, in accordance with an embodiment of the present invention;

FIGS. 14A-B are schematic illustrations of a coiled implant and a mechanical element disposed within a lumen of the implant, in accordance with an embodiment of the present invention;

FIG. 15 is a schematic illustration of a delivery tool comprising a motor, in accordance with an embodiment of the present invention;

FIG. 16 is a schematic illustration of an implant providing a scaffold for longitudinal rods, in accordance with an embodiment of the present invention;

FIGS. 17A-D are schematic illustrations of a delivery tool and a plurality of implants coupled thereto, in accordance with an embodiment of the present invention;

FIGS. 18A-E are schematic illustrations of a delivery tool and plurality of implants coupled thereto, in accordance with another embodiment of the present invention;

FIGS. 19A-B are schematic illustrations of a resorbable implant, in accordance with an embodiment of the present invention;

FIGS. 20A-D are schematic illustrations of a delivery tool coupled to two implants, in accordance with an embodiment of the present invention;

FIGS. 21A-F are schematic illustrations of a deflectable delivery tool to implant a plurality of implants, in accordance with an application of the present invention;

FIGS. 22A-C are schematic illustrations of a delivery tool and a curved implant being implanted in tissue surrounding the urethra, in accordance with an application of the present invention;

FIGS. 23A-B are schematic illustrations of a cross-section of the prostate showing a plurality of curved implants implanted in tissue surrounding the urethra, in accordance with an application of the present invention;

FIGS. 24A-B are schematic illustrations of a plurality of coiled implants implanted in the prostate, in accordance with an application of the present invention;

FIGS. 25A-B are schematic illustrations of a delivery tool comprising inflatable balloons and a plurality of coiled implants being implanted in prostate tissue, in accordance with an application of the present invention;

FIG. 26 is a schematic illustration of a plurality of screw implants implanted in the prostate, in accordance with an application of the present invention;

FIGS. 27A-D are schematic illustrations of a delivery tool and a plurality of coiled implants coupled to a wire, in accordance with an application of the present invention; and

FIGS. 28A-D are schematic illustrations of a rod, a coiled implant, and a delivery tool, in accordance with an application of the present invention.

### DETAILED DESCRIPTION OF THE EMBODIMENTS

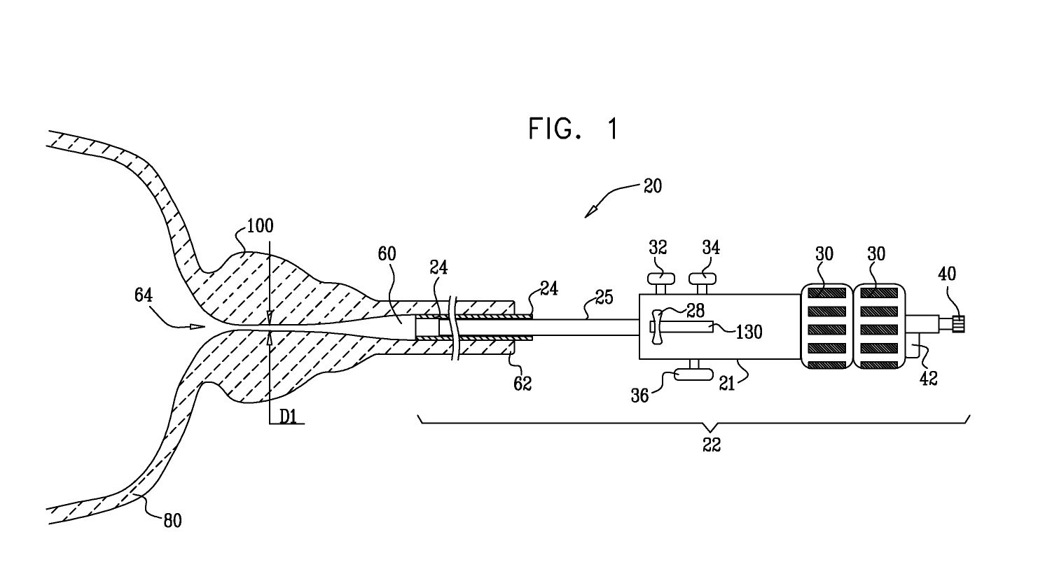

Reference is made to FIG. 1, which is a schematic illustration of a system 20 comprising a delivery tool 22 being introduced into a urethra 60 of a patient, in accordance with an embodiment of the present invention. Urethra 60 is constricted due to pressure exerted thereupon by a prostate 100 of the patient. Stenosis of urethra 60 by prostate 100 defines a diameter D1 at a bladder neck 64 of the patient and along a portion of urethra 60 surrounded by prostate 100. Untreated stenosis of urethra 60 by prostate 100 (e.g., responsively to benign prostate hyperplasia) often engenders acute urinary retention by bladder 80 of the patient, thus causing infrequent urination and ultimately, incontinence. Systems, methods, and apparatus described herein are configured to enlarge and expand the diameter of the constricted urethra and, in some embodiments, treat benign prostate hyperplasia.

An outer sheath 24 is advanced distally through a proximal end 62 of urethra 60 and toward bladder neck 64 of the patient. Outer sheath 24 expands urethra 60 as sheath 24 is distally advanced toward bladder 80 of the patient. Typically, outer sheath 24 is advanced via an introducer tube (not shown) having a rounded proximal end which facilitates atraumatic advancement of outer sheath 24 through urethra 60. Outer sheath 24 is advanced along urethra 60 prior to the advancement of delivery tool shaft 25, thus creating an open passageway for the subsequent insertion of delivery tool 22. Typically, outer sheath 24 is hollow and enables passage of tools through the urethra by providing a working channel of sheath 24. An imaging device (not shown), e.g., a fiberscope or a cystoscope, is advanced through outer sheath 24 into bladder 80. Bladder 80 and bladder neck 64 are examined prior to the introduction of delivery tool 22 into urethra 60 of the patient. The imaging device is typically flexible and bends 180 degrees in a proximal direction, facilitating visualization of a vicinity of bladder neck 64 of the patient.

Delivery tool 22 comprises a body 21 and a delivery tool shaft 25 which is advanced distally through outer sheath 24 toward bladder 80 of the patient. Typically, shaft 25 comprises a hollow lumen for passing substances and/or tools therethrough, such as but not limited to, medications, fiber optics, biopsy tools, optical devices (e.g., CCD) and/or other imaging devices.

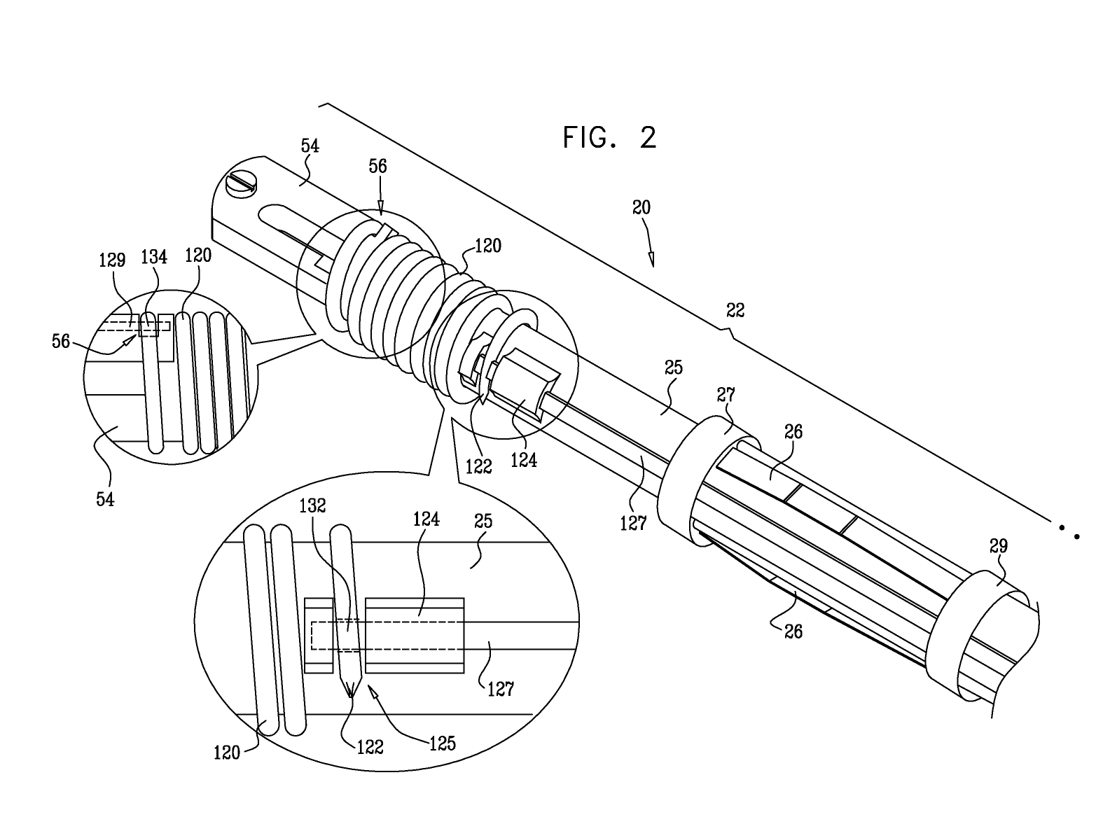

Reference is now made to FIG. 2, which is a schematic illustration of system 20 comprising a transurethrally implantable prostatic implant 120, which surrounds a distal end of shaft 25 of delivery tool 22, in accordance with an embodiment of the present invention. Typically, implant 120 comprises a radially-expandable implant, e.g., a coil or a helical implant. Implant 120 typically comprises a flexible biocompatible material, e.g., nitinol or silicone.

During transurethral advancement, implant 120 is disposed in a compressed state thereof between a proximal implant holder 124 and a distal implant holder 54. Typically, a distal end 126 and a proximal end 122 of implant 120 are each shaped to define a slit (134 and 132, respectively). Each slit is configured for passage of respective fastening devices therethrough. The fastening devices maintain the compressed state of implant 120 during advancement thereof into bladder 80 of the patient.

Proximal implant holder 124 is shaped to provide a latitudinal groove 125 for holding and securing proximal end 122 of implant 120. Additionally, proximal implant holder 124 is shaped to provide a longitudinal slit for advancement of a first elongate mechanical fastener 127 therethrough and subsequently through slit 132 of proximal end 122 of implant 120. Fastener 127 is advanced (a) through the longitudinal slit within holder 124, (b) subsequently through slit 132 of proximal end 122 of implant 120, and (c) back into a slit at a portion of holder 124 distal to groove 125.

Distal implant holder 54 comprises a similar securing mechanism as holder 124. Distal implant holder 54 is shaped to provide a latitudinal groove 56 at a proximal end thereof which holds and secures distal end 126 of implant 120 in a compressed state during advancement thereof. Additionally, distal implant holder 54 maintains coupling of implant 120 to tool 22 during implantation of implant 120. A second elongate mechanical fastener 129 is advanced (a) through a longitudinal slit within distal implant holder 54, (b) subsequently through slit 134 of distal end 126 of implant 120, and (c) back into a slit at a portion of distal implant holder 54 proximal to groove 56.

The securing and releasing of fasteners 127 and 129 are controlled remotely, by body 21 of delivery tool 22.

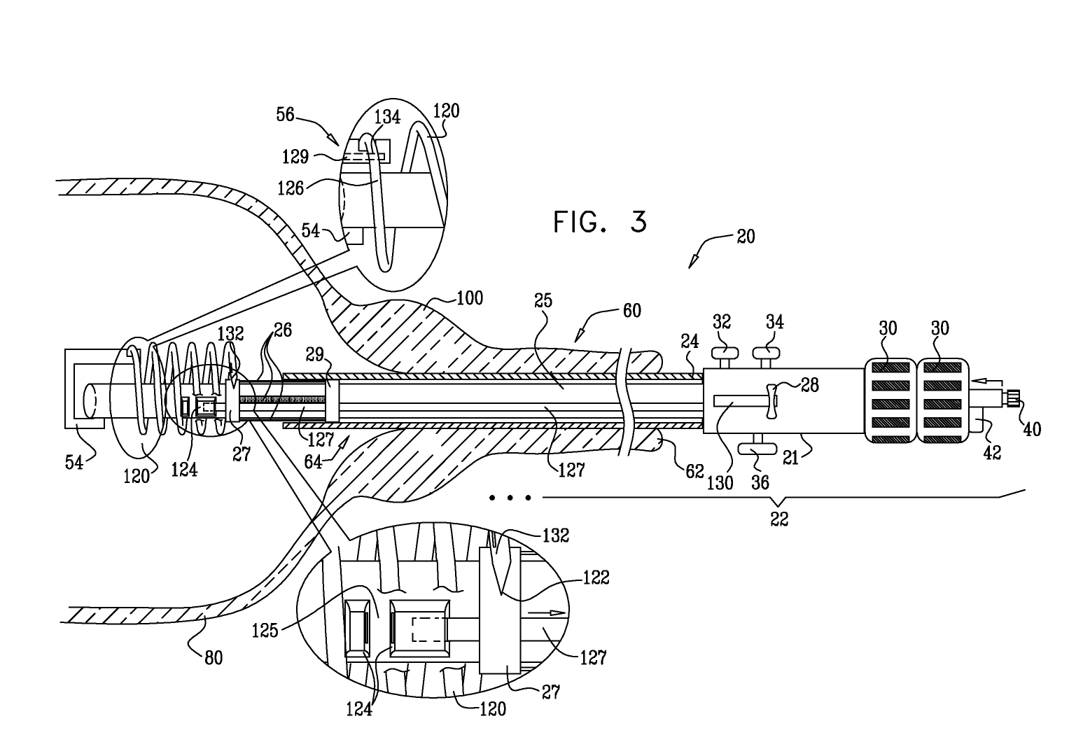

FIG. 3 shows implant 120 expanding from a compressed state thereof, in accordance with an embodiment of the present invention. The distal-most end of sheath 24 is disposed distally to bladder neck 64, facilitating proper placement within bladder 80 of any device passed through sheath 24, e.g., implant 120. Typically, outer sheath 24 is shaped to define a length which is shorter than a length of delivery tool shaft 25. Thus, once shaft 25 has been fully advanced through sheath 24, proximal end 122 of implant 120 is disposed distally with respect to the distal-most end of sheath 24. When proximal end 122 of implant 120 has sufficiently entered bladder 80 of the patient (e.g., as shown), proximal end 122 is released from proximal implant holder 124, allowing implant 120 to assume an expanded configuration.

Reference is again made to FIG. 1. Delivery tool 22 comprises a rotating element 30 at a proximal end thereof which is configured to facilitate implantation of implant 120 once the implant is inside bladder 80 of the patient. During the distal advancing of delivery tool shaft 25 toward bladder 80 of the patient, rotating element 30 is disposed adjacent to body 21 of tool 22, as shown.

Body 21 comprises one or more control elements 28 on a surface of tool 22 which enables a physician to control, from outside of the patient's body, one or more functional elements located at the distal end of delivery tool 22. Typically, but not necessarily, control elements 28 comprise rings for the physician to engage her fingers therethrough and push or pull on control elements 28. During advancement of delivery tool 22 within sheath 24, elements 28 are disposed in a distal orientation with respect to delivery tool 22, e.g., at the distal end of a slot 130 in delivery tool 22 (configuration not shown).

As shown in FIG. 3, once the distal end of delivery tool shaft 25 enters bladder 80, implant 120 is further pushed distally by pushing on a switch 42 disposed at a proximal end of body 21 of tool 22. Such pushing further facilitates that proximal end 122 of implant 120 is disposed distal to bladder neck 64 prior to implantation of implant 120 therearound. Control elements 28 are then pulled proximally with respect to delivery tool 22. Control elements 28 are coupled to a proximal end of fastener 127. In response to the pulling, a distal end of fastener 127 is pulled to a position that is proximal to proximal end 122 of implant 120, thereby releasing proximal end 122 of implant 120 and effecting radial expansion thereof. FIG. 3 shows system 20 immediately after control elements 28 have reached their proximal-most extent, decoupling fastener 127 from implant 120, but prior to the resultant radial expansion of the implant. During expansion of implant 120, distal end 126 of implant 120 remains coupled to distal implant holder 54.

To minimize the chance of physician error, tool 22 may comprise a distal lock 32, a proximal lock 34, and a release 36. Pulling and pushing of control elements 28 is restricted by locks 32 and 34. For example, when elements 28 are disposed distally with respect to tool 22, distal lock 32 automatically maintains the distal position of elements 28 such that elements 28 are not inadvertently pulled (resulting in premature expansion of implant 120 during advancement thereof). When proximal motion of control elements 28 is desired, the physician activates release 36, to release lock 32, allowing for such proximal motion of elements 28. Once disposed proximally with respect to tool 22, proximal lock 34 secures elements 28 in place, typically automatically.

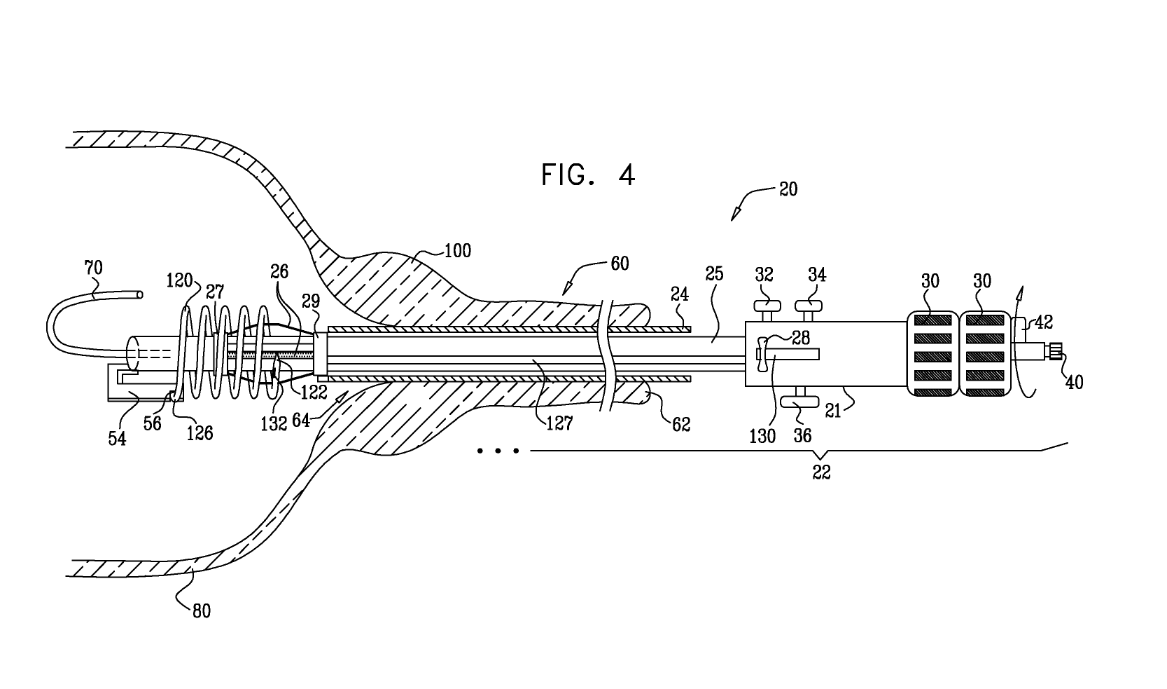

Reference is now made to FIG. 4, which is a schematic illustration of system 20 comprising expandable guiding elements 26, in accordance with an embodiment of the present invention.

Reference is now made to FIGS. 3 and 4. As shown in FIG. 3, during advancement of tool 22 through outer sheath 24, distal implant holder 54 is disposed in a configuration such that a distal portion thereof covers a lumen of shaft 25 of delivery tool 22. Switch 42 is oriented in a downward configuration indicative of the closed configuration of distal implant holder 54. As shown in FIG. 4, manually rotating switch 42 in an upward configuration, e.g., 180 degrees, rotates distal implant holder 54, thereby exposing the lumen of delivery tool shaft 25. Additionally, rotation of distal implant holder 54 positions implant 120 coaxially with respect to the urethra, such that implant 120 is properly corkscrewed symmetrically around the urethra. Imaging device 70 is then advanced through the lumen of shaft 25, and guides the subsequent implantation of implant 120. Imaging device 70 is configured to bend 180 degrees and rotate 360 degrees in order to image the implantation procedure.

Reference is now made to FIGS. 2 and 4. As shown in FIG. 2, expandable guiding elements 26 surround a portion of delivery tool shaft 25 proximal to implant 120. Distal and proximal ends of expandable elements 26 are each coupled to a first ring 27 and a second ring 29, respectively. Typically, first ring 27 is fixed to a portion of shaft 25 while second ring 29 is configured to slide distally and proximally along shaft 25. Alternatively, first ring 27 is configured to slide distally until a stopping element impedes continued distal motion of ring 27. Such distal motion of ring 27 facilitates positioning of ring 27 and distal portions of guiding elements 26 within the lumen of the implant prior to expansion of elements 26. During the advancing of delivery tool shaft 25 toward bladder 80, guiding elements 26 are typically pressed against the outer surface of shaft 25.

FIG. 4 shows deployment of expandable guiding elements 26 following expansion of implant 120. Distal pushing of control elements 28 slides ring 29 distally toward ring 27. The distal and proximal ends of expandable elements 26 are drawn toward one another, resulting in the radial expansion of expandable elements 26. Expandable guiding elements 26 expand such that they align with an inner surface of implant 120. Such alignment facilitates the guiding of implant 120 and the maintenance of a straight configuration thereof during the implantation procedure.

Typically, once implant 120 is fully disposed within bladder 80, body 21 of tool 22 is disposed adjacent to a proximal-most end of sheath 24. The implantation of implant 120 within prostate 100 begins when the physician distances body 21 from outer sheath 24, thereby shifting tool 22 proximally. Such shifting positions proximal end 122 of implant 120 in proximity with bladder neck 64 immediately prior to implantation of implant 120.

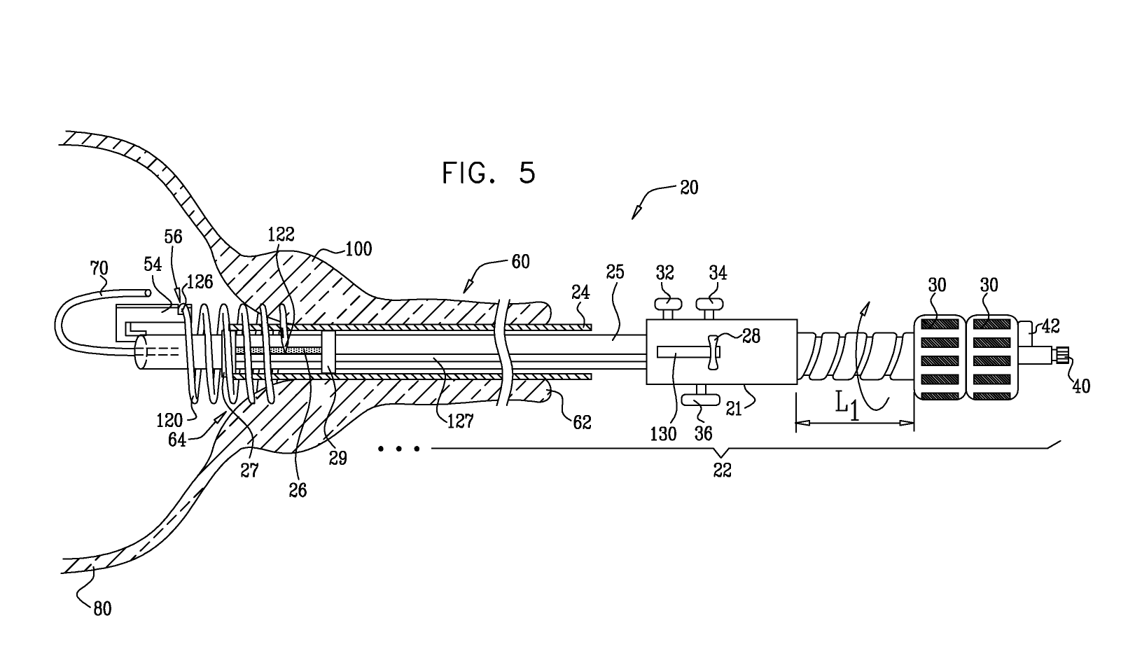

Reference is now made to FIG. 5, which is a schematic illustration of implant 120 of system 20 being partially implanted in prostate 100 of the patient, in accordance with an embodiment of the present invention. Upon expansion within bladder 80 of the patient, implant 120 is shaped to define an inner lumen diameter, e.g., 2.5 mm to 15 mm, typically larger than the non-constricted outer diameter of urethra 60.

Proximal end 122 of implant 120 is typically pointed and is configured to puncture tissue of prostate 100. In some embodiments, proximal end 122 is coupled to, e.g., soldered to or attached using any other applicable attachment means, a pointed needle which is configured to puncture tissue of the patient. Typically, the needle coupled to proximal end 122 comprises a rigid, biocompatible material, e.g., stainless steel, configured to configured to facilitate ongoing penetration of the implant as it is advanced through tissue of prostate 100. It is to be noted that the needle is shaped to define any suitable shape configured for cutting/penetrating tissue.

Following the puncturing of the tissue by proximal end 122 or, in some embodiments, the needle coupled thereto, implant 120 is further advanced proximally in the tissue of prostate 100, around urethra 60 of the patient. Counterclockwise rotation of rotating element 30 rotates and proximally retracts implant 120, thus corkscrewing implant 120 within tissue of prostate 100 surrounding urethra 60. Positioning of implant 120 within tissue of prostate 100 is typically guided by imaging element 70.

In some embodiments, as proximal end 122 (or a needle coupled thereto) of implant 120 is advanced through the tissue of the patient, it is configured to ablate the tissue. In such an embodiment, implant 120 may be coated with a substance, such as but not limited to, a medication (e.g., an antibiotic) or with an electrical insulator (e.g., Teflon). A portion of proximal end 122 of implant 120, i.e., one or more of the coils, may be energized to deliver RF energy, for example, to ablate tissue. In some embodiments, the portion of proximal end 122 of implant 120 is coupled to an electrode. Additionally or alternatively, the portion of proximal end 122 of implant 120 may be energized to provide ultrasound or thermal energy (e.g., heating or cooling).

In some embodiments, the implant 120 comprises a hollow, helical implant shaped to define a helical lumen and at least one hole, e.g., a plurality of holes, at the proximal end thereof. In such an embodiment, a fluid, e.g., saline, is injected at high pressure through the lumen of the hollow, helical implant and externally to the implant via the at least one hole in order to cut tissue near the proximal tip of the implant as it advances through the tissue.

In some embodiments, the hollow, helical implant is configured for passage through its lumen and through the hole at the proximal end thereof, of a laser fiber to ablate tissue in the path of the implant as it is advanced therethrough. In some embodiments, an insulated RF transmitting wire (i.e., having a non-insulated transmitting-tip) is advanced through the helical lumen of the hollow implant.

In some embodiments, the hollow, helical implant is configured for passage through its lumen of a fluid (configuration described hereinbelow with reference to FIG. 12). The hollow, helical implant is shaped to define holes (e.g., typically toward the proximal end of the implant) for release of the fluid externally to the implant. In some embodiments, the fluid comprises a lubricant which passes externally to the implant via the holes defined thereby in order to reduce a frictional force between the tissue and the implant.

Expandable guiding elements 26 guide the initial implantation (e.g., longitudinal motion of 6 mm to 11 mm in a proximal direction) of proximal end 122 of implant 120 around urethra 60. Control elements 28 are then pulled proximally, thereby sliding ring 29 proximally such that guiding elements 26 are pressed once again against the outer surface of shaft 25 (alignment shown in FIG. 2). As shown in FIG. 5, control elements 28 are disposed in a proximal orientation with respect to body 21 of delivery tool 22 indicating a retracted state of guiding elements 26.

Clockwise rotation of rotating element 30 retracts shaft 25, thereby retracting distal implant holder 54 attached to distal portion 126 of implant 120. In response to the retracting, distal implant holder 54 helps corkscrew implant 120 into tissue of prostate 100 by applying to implant 120 a force in the proximal direction. During the clockwise rotation, rotating element 30 is distanced from body 21 of delivery tool 22 by a distance L1. L1 is typically smaller than a maximal distance between rotating element 30 and body 21 of tool 22, thus indicating partial implantation of implant 120 around urethra 60 of the patient.

Following initial partial implantation of implant 120 and alignment of expandable guiding elements 26 along shaft 25, implant 120 is further advanced proximally through prostate 100, around urethra 60 of the patient. Once implant 120 is fully implanted in prostate 100, distal end 126 is decoupled from distal implant holder 54 by retracting fastener 129 from slit 134 at distal end 126 of implant 120. Fastener 129 is controlled by a control element 40, which is disposed at a proximal end of body 21 of delivery tool 22. Pulling on element 40 retracts fastener 129 from slit 134, thereby releasing implant 120 from holder 54. Switch 42 is then rotated in a downward direction, e.g., 180 degrees (not shown), restoring the original position of distal implant holder 54, enabling subsequent passage thereof through sheath 24. Imaging device 70 is then straightened and extracted from bladder 80 via sheath 24.

Typically, as implant 120 is advanced through tissue of prostate 100, tissue of prostate 100 applies a frictional force to implant 120. In some embodiments, in order to reduce the effect of the frictional force applied to implant 120, implant 120 is coated with a low-friction coating, e.g., PTFE (Teflon), MoST, ADLC or the like. In some embodiments, the implant surface is polished, e.g., electro-polished, mechanically polished, or other, to reduce friction as implant 120 is advanced through the tissue of the patient.

In some embodiments, implant 120 comprises a helical implant comprising a plurality of coils which are helically surrounded by a sheath coupled to a tube for passage therethrough of a lubricant into the sheath surrounding the implant (configuration shown hereinbelow with reference to FIG. 12). Typically, a lubricant is passed through the sheath surrounding implant 120. In such an embodiment, the sheath surrounding the implant is shaped to define holes (e.g., typically toward proximal end 122 of implant 120) for release of the lubricant externally to implant 120. The lubricant reduces a frictional force between the tissue of prostate 100 and implant 120. In some embodiments, implant 120 itself is a hollow, helical implant defining a helical lumen therein configured for passage of lubricant therethrough. The hollow, helical implant is shaped to define holes (e.g., typically toward proximal end 122 of implant 120) for release of the lubricant externally to implant 120. In such an embodiment, the implant is coupled to the tube for delivering the lubricant thereto, typically without the use of a sheath.

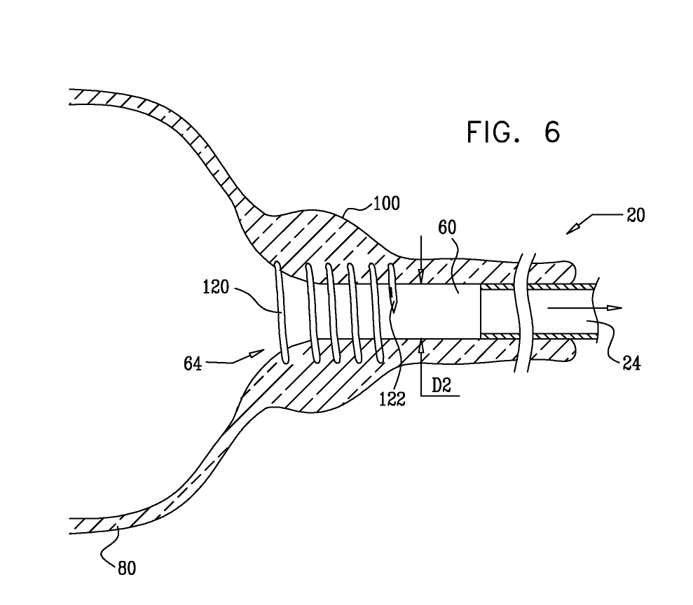

Reference is now made to FIG. 6, which is a schematic illustration of implant 120 implanted within prostate 100 of the patient, in accordance with an embodiment of the present invention. Once implant 120 is implanted within prostate 100, delivery tool shaft 25 and outer sheath 24 are extracted from within urethra 60. Following implantation of implant 120 within prostate 100, a post-operative diameter D2 of the portion of urethra 60 at prostate 100 is larger than diameter D1 of the portion of urethra 60 prior to implantation of implant 120. Implant 120 is generally rigid relative to the rigidity of the prostate. The implant thus supports the urethral tissue, minimizing restenosis of urethra 60 should prostate 100 continue to enlarge.

Typically, implant 120 is selected to provide a length according to the needs of a given patient. A length of prostate 100 is measured prior to the implantation procedure such that an implant of a suitable length is selected. Typically, the end-to-end length of the coiled implant ranges from between 2.5 cm and 7 cm, to accommodate a prostate length of between 3 and 8.6 cm, respectively.

Typically, implant 120 supports prostatic tissue 100 surrounding urethra 60 without touching the urethral epithelium or other delicate tissue, and enlarges the lumen in urethra 60.

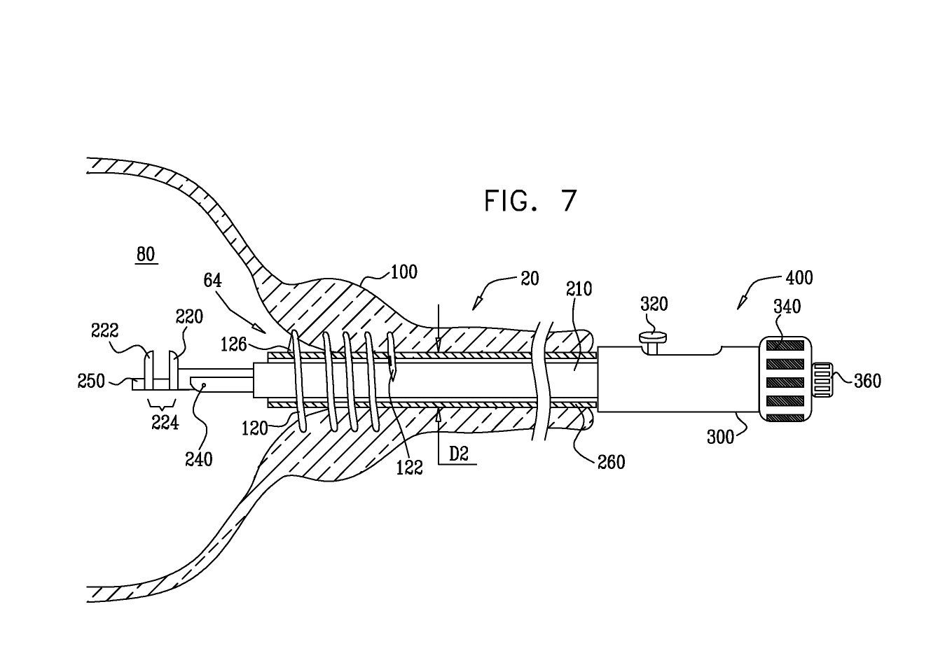

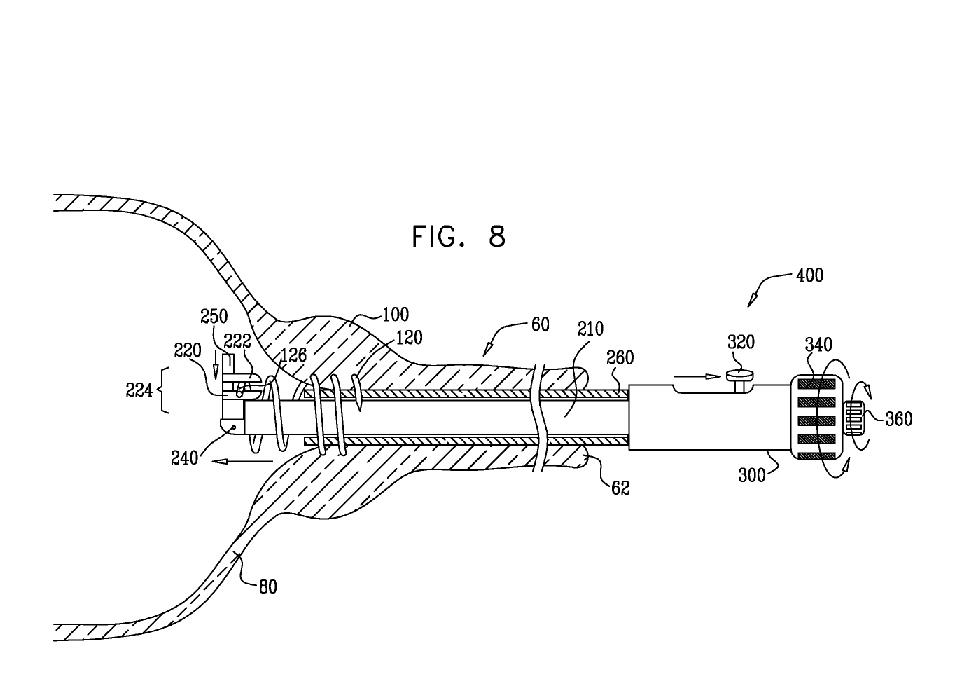

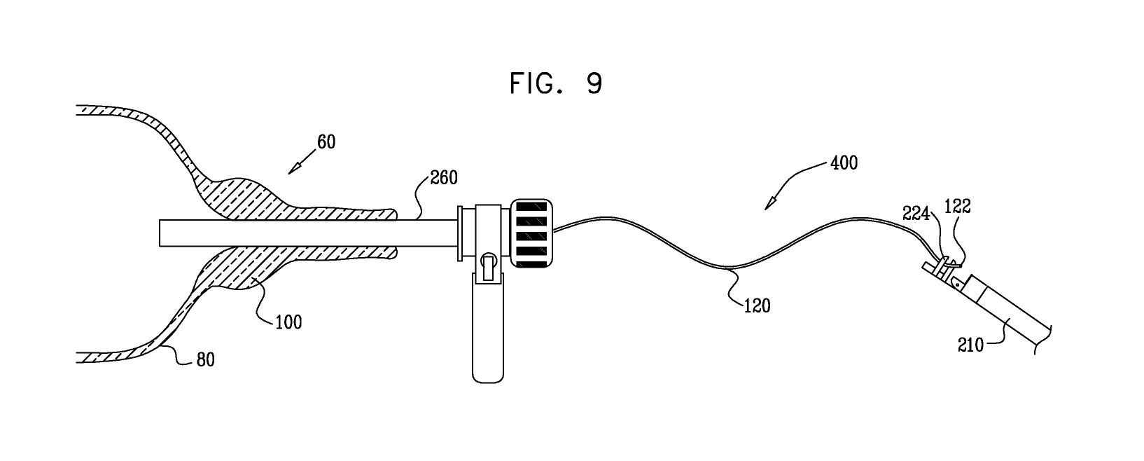

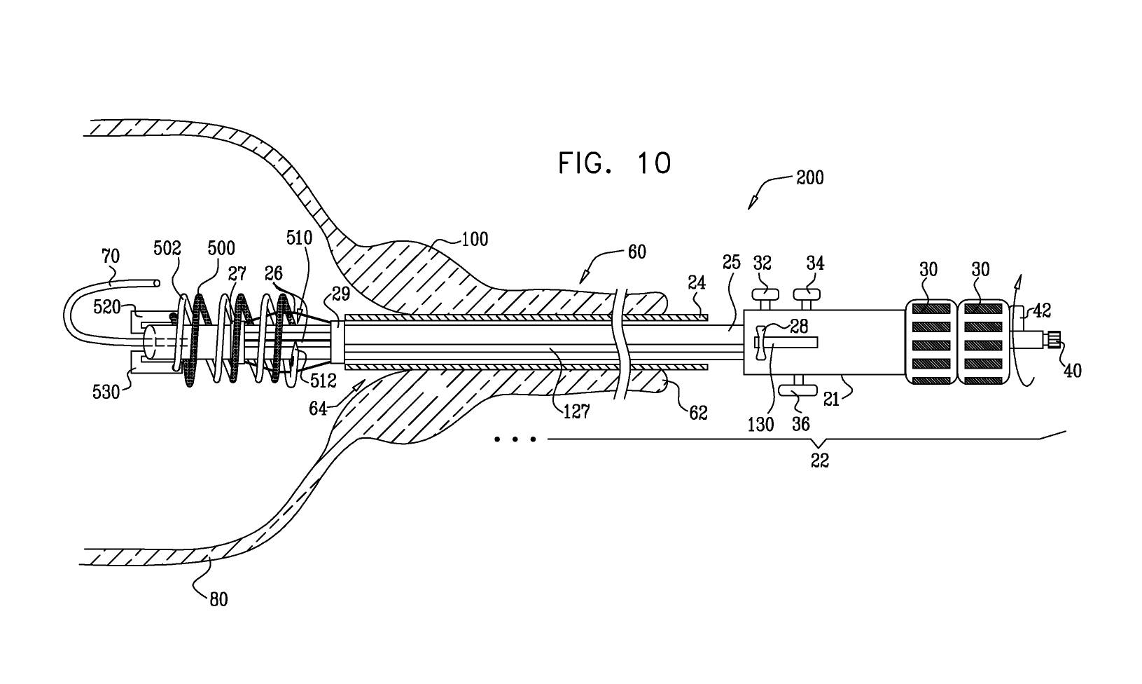

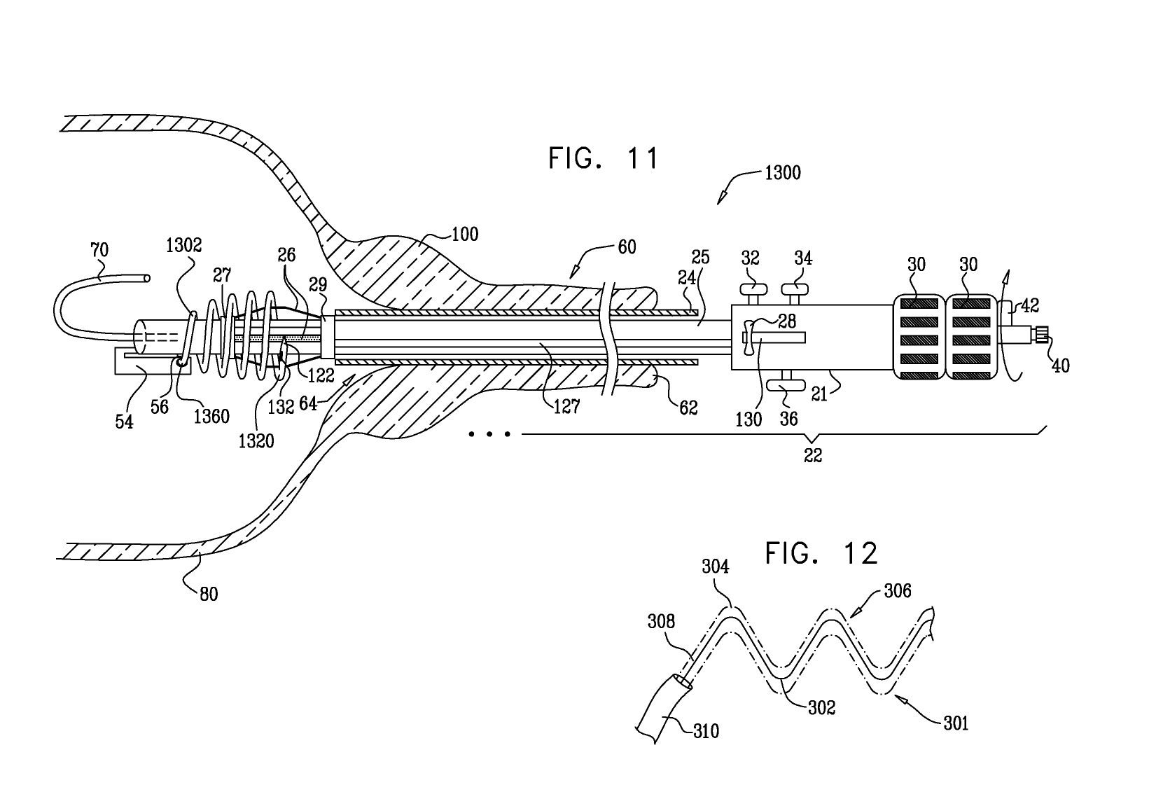

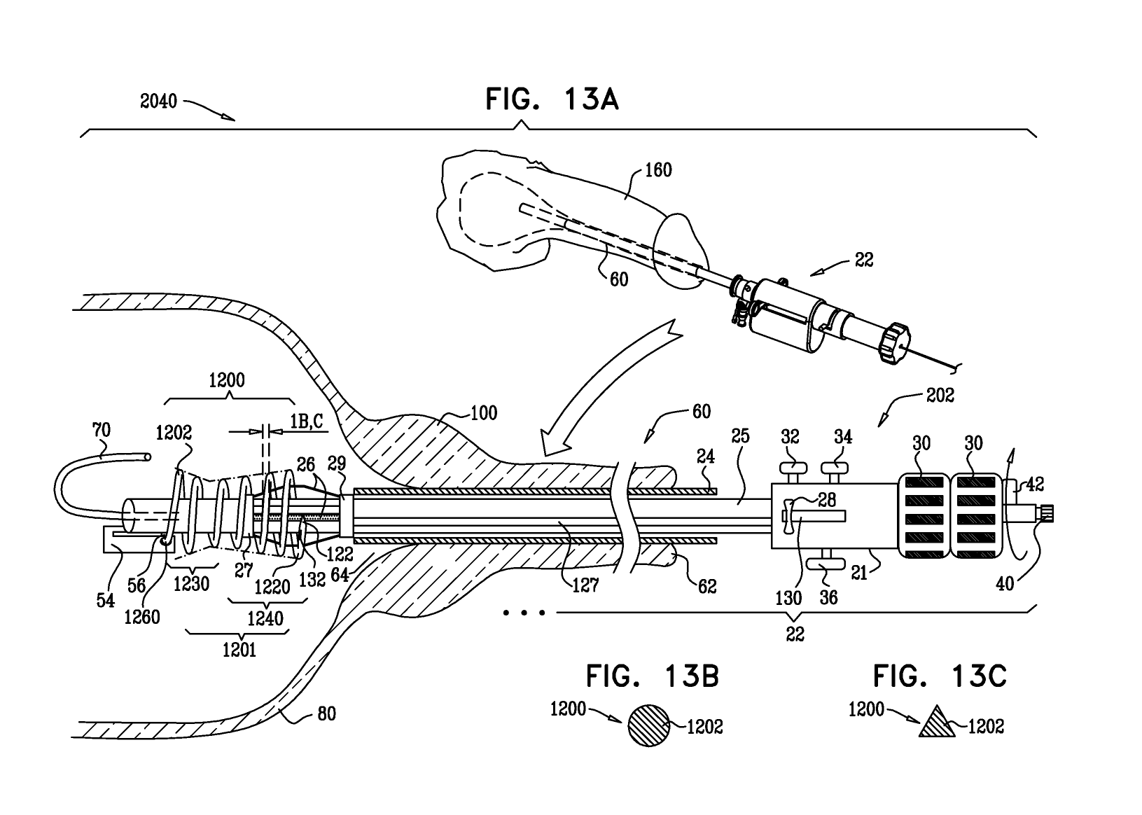

FIGS. 7 and 8 show a system 400 comprising an extraction tool 300 configured to remove implant 120 from prostate 100, in accordance with an embodiment of the present invention. An outer sheath 260 is advanced distally through proximal end 62 of urethra 60 and toward bladder neck 64 of the patient. Subsequently, a resection tool, e.g., a resectoscope (not shown), is advanced through sheath 260. The resection tool removes tissue surrounding distal end 126 of implant 120, thereby exposing a portion of implant 120 and enabling engaging thereof by extraction tool 300.

Typically, extraction tool 300 comprises a shaft 210 which is coupled at a distal end thereof to a mechanically adjustable clamp 224 via a hinge 240. Clamp 224 is advanced through sheath 260 into bladder 80 in an “extended” configuration with respect to hinge 240 (as shown in FIG. 7), and later assumes a “flexed” configuration with respect to the hinge, which enables clamp 224 to engage implant 120 (as shown in FIG. 8). An optical guide 250, e.g., a CCD, CIS, or CMOS sensor or an optical fiber-based system, guides the engaging and subsequent extraction of implant 120.

As shown in FIG. 8, a control element 320 is disposed along extraction tool 300 and enables a physician to control, from a location outside the body of the patient, various mechanical functions being performed at the distal end of tool 300. By proximal pulling of element 320, clamp 224 is flexed at hinge 240 with respect to shaft 210.

Additionally, extraction tool 300 comprises a proximal rotating element 360 and a distal rotating element 340. Proximal rotating element 360 regulates a distance between an upper jaw 222 and a lower jaw 220 of clamp 224. Upon an indication from imaging device 250 that clamp 224 surrounds a portion of implant 120, proximal rotating element 360 is rotated in a clockwise direction in order to reduce the distance between jaws 220 and 222 thus facilitating clamping of implant 120 by clamp 224.

The extraction process begins when clamp 224 engages a distal portion of implant 122. Distal rotating element 340 is rotated in a counterclockwise direction, i.e., in a direction opposite the direction used in the implantation procedure. Such rotation of element 340 moves implant 120 distally by rotating implant 120 about a longitudinal axis of extraction tool 300.

Once implant 120 is extracted fully from within prostate 100, jaws 220 and 222 are released from the distal portion of implant 120 by counterclockwise rotation of proximal rotating element 360. Element 320 is pushed distally, restoring clamp 224 to an extended configuration with respect to hinge 240. Clamp 224 is then pulled proximally, such that jaws 220 and 222 are aligned with a proximal portion of implant 120. Element 320 is again pulled proximally, and rotating element 360 is once again rotated in a clockwise direction such that jaws 220 and 220 are drawn together and engage the proximal end of implant 120 (configuration not shown).

FIG. 9 is a schematic illustration of extraction tool 300 extracting implant 120, in accordance with an embodiment of the present invention. The proximal end of the coiled implant is pulled in a proximal direction through a lumen of shaft 210. Due to the relative flexibility and elasticity of implant 120 compared to extraction tool 300, pulling of implant 120 through sheath 260 enables implant 120 to assume an elongated, generally straightened configuration.

In some embodiments, two clamps are used in order to extract the implant from the bladder of the patient. A distal clamp typically extracts the implant from the prostate. Subsequent to the extraction, a proximal clamp is advanced into the bladder of the patient and engages the proximal end of the implant. The proximal clamp is used to pull the implant through the outer sheath as the distal clamp remains at a fixed distance within the bladder of the patient. Such configuration of the distal clamp with respect to the proximal clamp enhances stability of the extraction procedure by maintaining the distal end of the implant within the bladder as the proximal end is being pulled by the proximal clamp into a straight configuration and ultimately outside the body of the patient.

Reference is now made to FIG. 10, which is a schematic illustration of a system 200 comprising first and second transurethrally implantable prostatic implants 500 and 502, which surround the distal end of shaft 25 of delivery tool 22, in accordance with an embodiment of the present invention. Typically, implants 500 and 502 comprise helical, radially-expandable implants, e.g., coils, each having an inner diameter of at least 2.5 mm, e.g., between 2.5 mm and 15 mm. The respective diameters of the inner lumens of implants 500 and 502 enable implants 500 and 502 to be implanted in tissue surrounding urethra 60. Typically, the respective diameters of implants 500 and 502 are the same. Implants 500 and 502 typically comprise a flexible biocompatible material, e.g., nitinol or silicone.

First implant 500 comprises a pointed proximal end 510, and second implant 502 comprises a pointed proximal end 512. In some embodiments, proximal ends 510 and 512 are each coupled to, e.g., soldered to, a respective pointed tip, e.g., a needle.

Typically, the needles coupled to each proximal end 510 and 512 comprise a generally rigid, biocompatible material, e.g., stainless steel, and are configured to provide strength to implants 500 and 502, respectively, to facilitate their puncture of and advancement through tissue of prostate 100.

During transurethral advancement, implants 500 and 502 are disposed in a compressed state thereof. In some embodiments, implants 500 and 502 are compressed between respective proximal and distal implant holders 520 and 530. Typically, distal implant holders 520 and 530 function similarly to distal implant holder 54 as described hereinabove with reference to FIGS. 2-5. Typically, the distal and proximal ends 510 and 512 of implants 500 and 502, respectively, are each shaped to define a slit. Each slit is configured for passage of respective fastening devices therethrough. The fastening devices maintain the compressed state of implants 500 and 502 during advancement thereof into bladder 80 of the patient. Once delivery tool 22 positions implants 500 and 502 in bladder 80, the fastening devices are released and implants 500 and 502 are allowed to expand to assume the configuration shown.

As shown, implants 500 and 502 are disposed in a relative spatial configuration in which implants 500 and 502 are coaxially disposed and rotationally offset 180 degrees with respect to each other, by way of illustration and not limitation. Additionally, a longitudinal position of implant 500 overlaps at least in part (e.g., entirely, as shown) a longitudinal position of implant 502. Implants 500 and 502 may be rotationally offset at any given angle with respect to each other. It is to be noted that although two implants are shown, any suitable number of implants may be corkscrewed into tissue of prostate 100. For example, three or four longitudinally-overlapping coiled implants may be coaxially disposed and rotationally offset 120 or 90 degrees with respect to each other, respectively.

Typically, implants 500 and 502 are corkscrewed at the same time into tissue of prostate 100. During implantation:

proximal end 510 of the first implant 500 punctures the tissue of prostate 100 at a first location thereof, and

proximal end 512 of second implant 502 punctures the tissue of prostate 100, at a location 180 degrees from the first location.

The scope of the present invention includes sequentially implanting first and second implants 500 and 502. Delivery tool 22 is coupled to first implant 500 and delivers implant 500 to within bladder 80 and allows implant 500 to expand, as described hereinabove in FIGS. 1-4, with reference to the delivering and expanding of implant 120 within bladder 80. (As appropriate, delivery tool 22 may be sold already coupled to first implant 500.) First implant 500 punctures the tissue at a first location and is fully advanced into prostate 100 by delivery tool 22, as described hereinabove in FIGS. 5-6, with reference to the implanting of implant 120 within prostate 100.

Once first implant 500 is implanted, delivery tool 22 is removed from the patient, is coupled to second implant 502, and is reintroduced within urethra 60 of the patient. (Alternatively, another delivery tool 22 coupled to implant 502 is used in the following steps.) Second implant 502 is advanced into bladder 80 of the patient, is allowed to expand within bladder 80, as described hereinabove in FIGS. 1-4, with reference to the delivering and expanding of implant 120 within bladder 80. Second implant 502 then punctures the tissue of prostate 100 at a second location which is rotationally offset 180 degrees from the first location. Second implant 502 is corkscrewed into the tissue (as described hereinabove in FIGS. 5-6, with reference to the implanting of implant 120 within prostate 100). Second implant 502 is implanted coaxially with respect to a position of the implanted first implant 500. Second implant 502 is advanced fully through the tissue, until it is disposed coaxially and is rotationally offset by 180 degrees with respect to first implant 500.

For either embodiment in which implants 500 and 502 are implanted simultaneously or sequentially, once implanted, implants 500 and 502 are configured to assume the relative spatial configuration, as shown and as described hereinabove. Typically, once implanted, implants 500 and 502 maintain substantially the same spatial relationship as shown in FIG. 10, i.e., coaxially disposed, longitudinally overlapping, and rotationally offset by 180 degrees with respect to each other.

In order to minimize the frictional force of prostate 100 on each implant 500 and 502 during implantation:

1) when implanted, the end-to-end respective lengths of each of the coiled implants range from between 2.5 cm and 7 cm, to accommodate a prostate length of between 3 cm and 9 cm, respectively, and

2) in accordance with the lengths of implants 500 and 502 in the abovementioned range, implants 500 and 502 are each shaped to define a pitch of between 8 mm and 23 mm, respectively.

For example, each of implants 500 and 502 may have an end-to-end length of about 4.5-5.5 cm and a pitch of about 14-16 mm.

The scope of the present invention includes the implantation of any suitable number of coiled implants around the urethra of the patient. For example, when one coiled implant is implanted in the tissue, the coiled implant may have a length of 4.5-5 cm and a pitch of approximately 8 mm. When first and second coiled implants (e.g., implants 500 and 502, as shown) are configured to be coaxially disposed and rotationally offset 180 degrees with respect to each other, each coiled implant 500 and 502 has a length of 4.5-5 cm and a pitch of approximately 16 mm (i.e., twice that indicated for an embodiment in which one coiled implant is implanted). In this manner, when the respective longitudinal positions of the implants are overlapped, and the implants are rotationally offset and coaxially disposed within tissue of prostate 100, the average effective pitch between adjacent coils of the coaxially disposed first and second coiled implants 500 and 502 is approximately 8 mm.

Typically, a pitch of each coiled implant is directly proportional to the number of coiled implants configured to be coaxially disposed when implanted in tissue of the patient. For example, when one coiled implant is implanted in the tissue, the coiled implant may have a length of 3-5 cm and a pitch of approximately 3-9 mm. When first and second coiled implants are configured to be coaxially disposed and rotationally offset 180 degrees with respect to each other (e.g., when implanted in tissue), each coiled implant has a length of 3-5 cm and a pitch of approximately 6-18 mm, such that when the respective longitudinal positions of the implants are overlapped, and the implants are coupled together by being coaxially disposed, the effective average pitch between adjacent coils of the coaxially disposed first and second coiled implants is approximately 3-9 mm.

The total frictional force of the tissue of prostate 100 on any coiled implant during implantation is generally inversely related to the pitch and the length of the coil that is being implanted. That is, a small-pitch coiled implant has an along-the-coil length, i.e., the length of the wire when the coil is straightened, that is larger than an along-the-coil length of a high-pitch coiled implant. Thus, the overall frictional force applied to a small-pitch coiled implant is larger than the overall frictional force applied to a large-pitch coiled implant, because the frictional force applied to a small-pitch coiled implant is applied along a larger coil length, i.e., a larger cumulative surface area. Thus, as each of first and second coiled implants 500 and 502 is implanted within prostate 100, e.g., simultaneously or sequentially, the force needed in order to overcome the frictional force applied to each coiled implant 500 and 502 is smaller in comparison to the force applied to a coiled implant having a pitch similar to the average pitch of the combined first and second coiled implants 500 and 502. By reduction of the frictional force applied by the prostate to the implant during implantation, any undesired deformation of the portion of the implant that has not yet entered the prostate is reduced.

Additionally, the higher-pitch implant is characterized as being stronger and more rigid in comparison to the small-pitch coiled implants.

FIG. 11 shows a system 1300 comprising a prostatic implant 1302, which surrounds the distal end of shaft 25 of delivery tool 22, in accordance with an embodiment of the present invention. Typically, implant 1302 is shaped to define a conically-shaped implant comprising a proximal coil 1320 having a larger diameter than a distal coil 1360. Typically, the respective diameters of adjacent coils decrease from proximal coil 1320 to distal coil 1360.

Prior to advancement of implant 1302 through urethra 60, delivery tool 22 is coupled to implant 1302 in a compressed state thereof. Delivery tool 22 maintains the compressed state of implant 1302 as it is advanced through urethra 60 and into bladder 80. Once within bladder 80, implant 1302 is allowed to expand, as described hereinabove in FIGS. 1-4 with reference to the delivering and expanding of implant 120 within bladder 80. Pointed proximal end 122 of coil 1320 punctures the tissue of prostate 100 and is fully advanced into prostate 100 by delivery tool 22, as described hereinabove in FIGS. 5-6 with reference to the implanting of implant 120 within prostate 100. Once implant 1302 is implanted, delivery tool 22 is removed from the patient.

As proximal coil 1320 of implant 1302 is advanced through the tissue of prostate 100, the tissue applies a frictional force on the proximal coils of coiled implant 1302. In an attempt to continue corkscrewing into the tissue, the tissue exerts an increasingly larger cumulative frictional force on the increasing number of coils that are within the prostate. In response to the frictional force applied to the intra-prostate coils as they are corkscrewed into the tissue, the distal coils have a tendency to expand radially, such that the respective diameters of the distal coils are generally similar to the respective diameters of the proximal coils. In this manner, the overall outline of the entire implant when it has finished being inserted into the prostate tends to be generally rectangular (i.e., coils of same radius), rather than conical.

Once implanted, the distal coils maintain their expanded diameters such that implanted implant 1302 resembles implanted implant 120 as shown in FIG. 5.

FIG. 12 shows an implant system 301 comprising a helical implant 302 helically surrounded by a sheath 304, in accordance with an embodiment of the present invention. Typically, implant system 301 is advanced toward the bladder and is implanted around the urethra, as described hereinabove in FIGS. 1-6 with reference to the delivering and implantation of implant 120. Helical implant 302 is shaped to define a proximal end (not shown for clarity of illustration) comprising a pointed distal tip configured to puncture tissue of the prostate and facilitate ongoing penetration of implant system 301 within the tissue. Typically, the proximal end of helical implant 302 extends proximally from a proximal-most end of sheath 304 in order to facilitate unobstructed penetration of system 301 through tissue of the patient.

Typically, sheath 304 is shaped to define a plurality of holes 306 and is coupled at a distal end 308 thereof to a tube 310. Sheath 304 is fixedly attached to implant 302 at a site distal to the proximal end of implant 302 and is shaped to provide a helical lumen surrounding helical implant 302. Fluid is injected via tube 310 through the lumen of sheath 304. Holes 306 are configured for release of the fluid externally to implant system 301. In some embodiments, the fluid comprises a lubricant which passes externally to implant system 301 via holes 306 in order to reduce a frictional force between the tissue and implant system 301.

In some embodiments, sheath 304 is shaped to define at least one hole at the proximal end thereof (configuration not shown for clarity of illustration). In such an embodiment, the fluid may comprise saline which is injected at high pressure through the lumen sheath 306 and externally to implant system 301 via the at least one hole in the proximal end of sheath 304 in order to cut tissue near the proximal tip of implant 302 as it advances through the tissue. It is to be noted that the scope of the present invention includes the use of the high-pressure fluid to cut tissue independently of or in combination with cutting tissue using helical implant 302.

In some embodiments, implant 302 comprises a helical implant comprising a plurality of coils which are helically surrounded by a sheath (i.e., if the helical implant were to be pulled straight, the implant would be relatively-tightly enclosed within the sheath, analogously to a normal insulated wire (the implant) surrounded by a plastic insulator (the sheath)). The sheath is coupled at one end thereof to a tube for passage therethrough of a lubricant into the sheath surrounding the implant. In such an embodiment, the sheath surrounding the coils of the implant is shaped to define holes (e.g., toward the proximal end of the implant), for release of the lubricant externally to the implant. The lubricant, in turn, reduces the frictional force between the tissue and the implant.

In some embodiments, the hollow, helical lumen is configured for passage therethrough of a laser fiber to ablate tissue in the path of the implant as it is advanced therethrough. In some embodiments, an insulated RF transmitting wire having a non-insulated transmitting tip is advanced through the helical lumen of the hollow implant. It is to be noted that the scope of the present invention includes the use of the laser fiber and/or the RF wire independently of or in combination with helical implant 302.

In some embodiments, sheath 304 is shaped to define holes 304 only at the proximal end of the implant 302.

In some embodiments, helical implant 302 itself is a hollow, helical implant defining a helical lumen therein. Typically, the hollow, helical implant is functionally and structurally similar to and has the properties of sheath 304. In such an embodiment, the hollow, helical implant is typically implanted independently of sheath 304. In such an embodiment, the hollow, helical implant is coupled directly to tube 310.

Reference is now made to FIGS. 7-12. It is to be noted that the scope of the present invention includes use of extraction tool 300 (FIGS. 7-9) for extracting implants 500 and 502 (FIG. 10), 1302 (FIG. 11), 302 (FIG. 12), and any other implant described herein.

Reference is still made to FIGS. 7-12. In some embodiments of the present invention, a proximal clamp and a distal clamp are used in order to extract implants 120, 500, 502, 1302, 302, and/or any other implant described herein from prostate 100 and bladder 80 of the patient. The distal clamp typically extracts the implant from the prostate (as described hereinabove with reference to clamp 224). Subsequent to the extraction, the proximal clamp is advanced into bladder 80 of the patient and engages the proximal end of the implant (in a manner as described hereinabove with respect to clamp 224). The proximal clamp is used to guide the implant through outer sheath 230 as the distal clamp remains within bladder 80 of the patient.

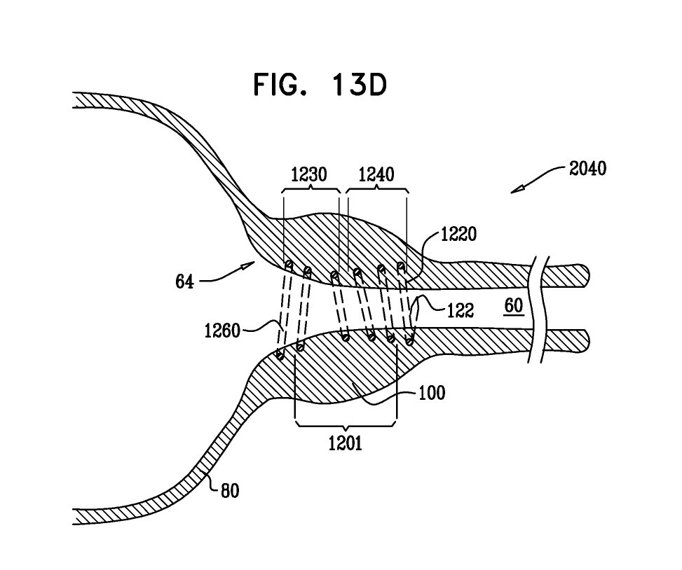

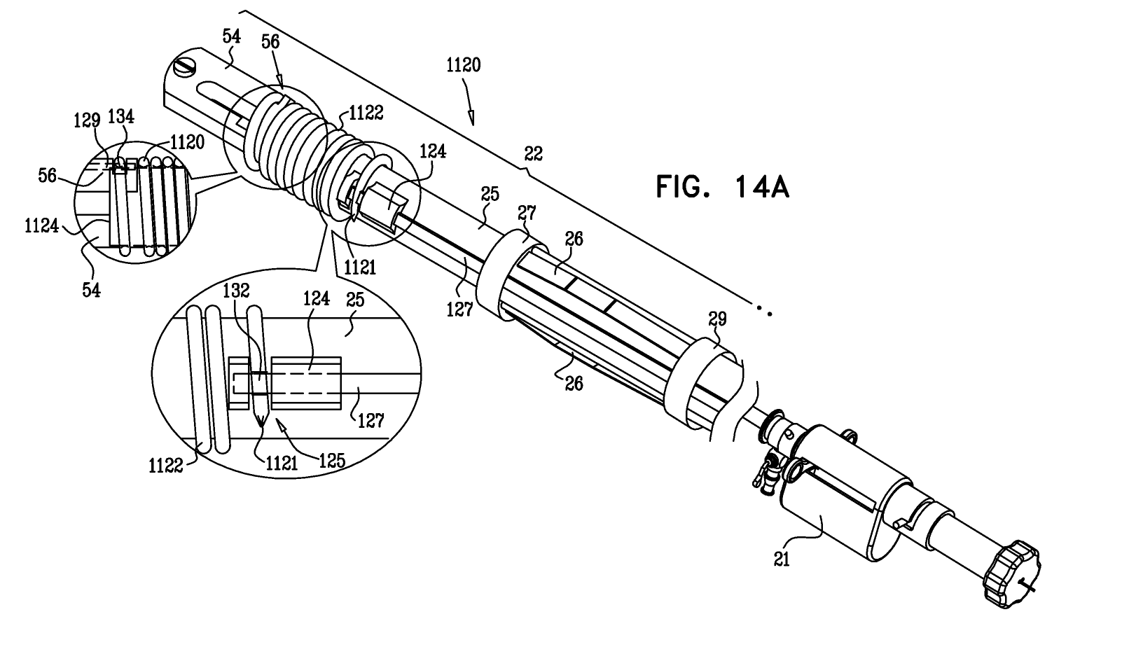

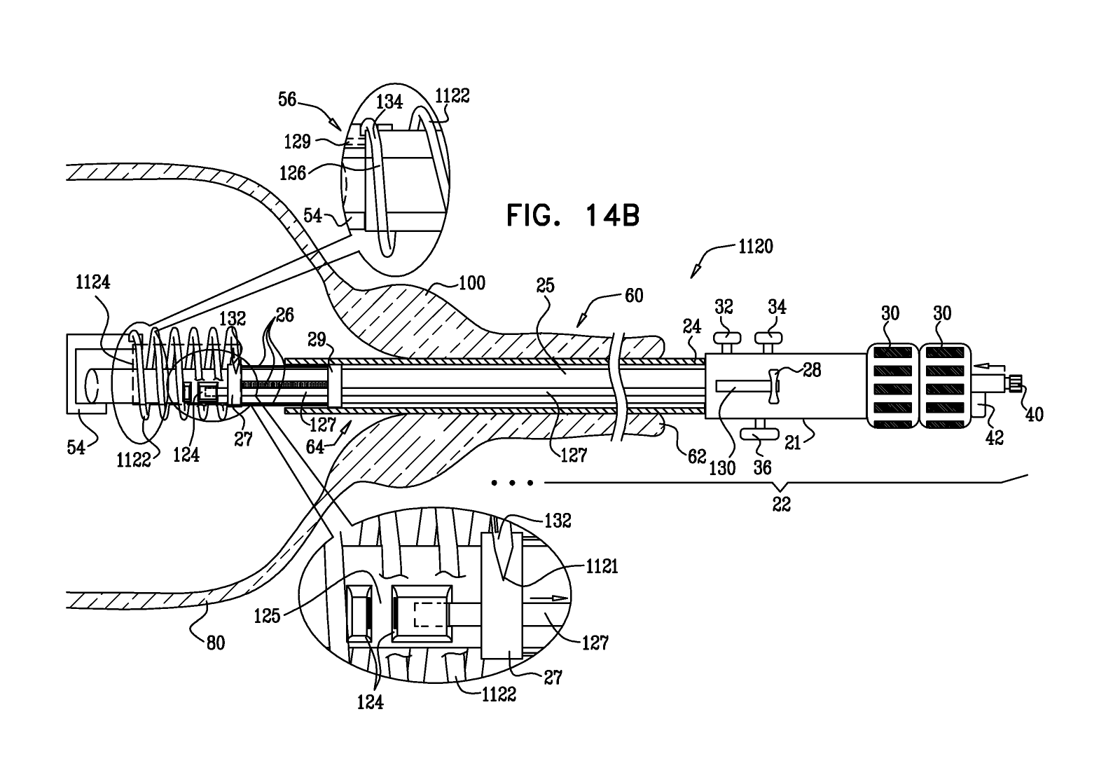

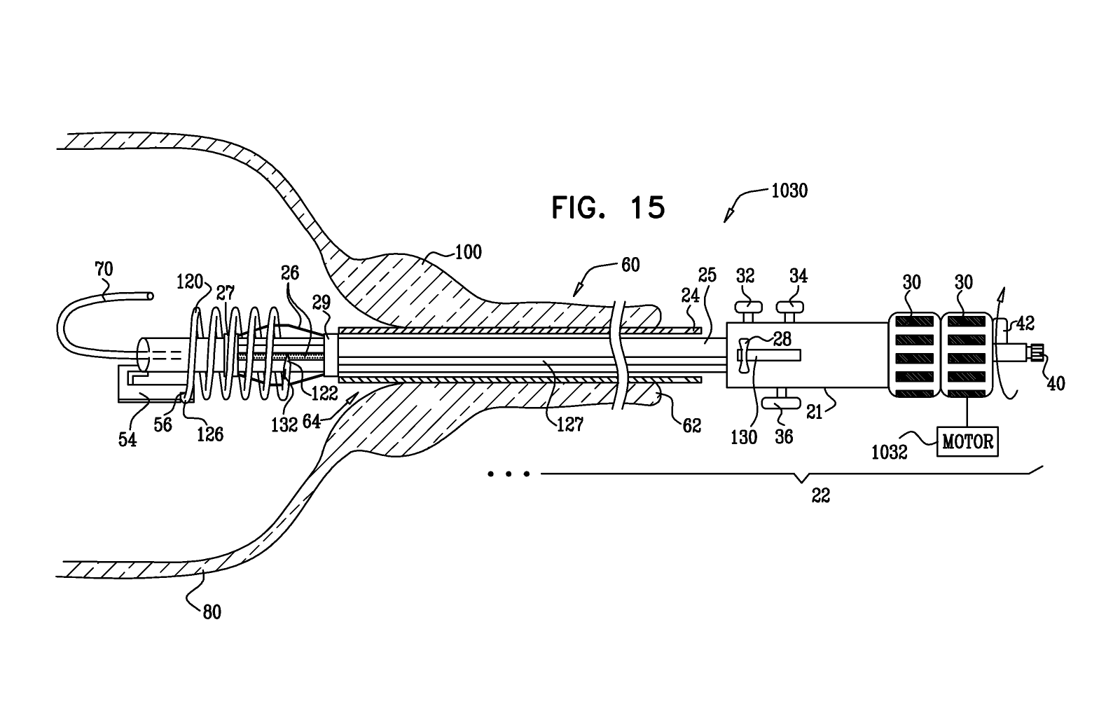

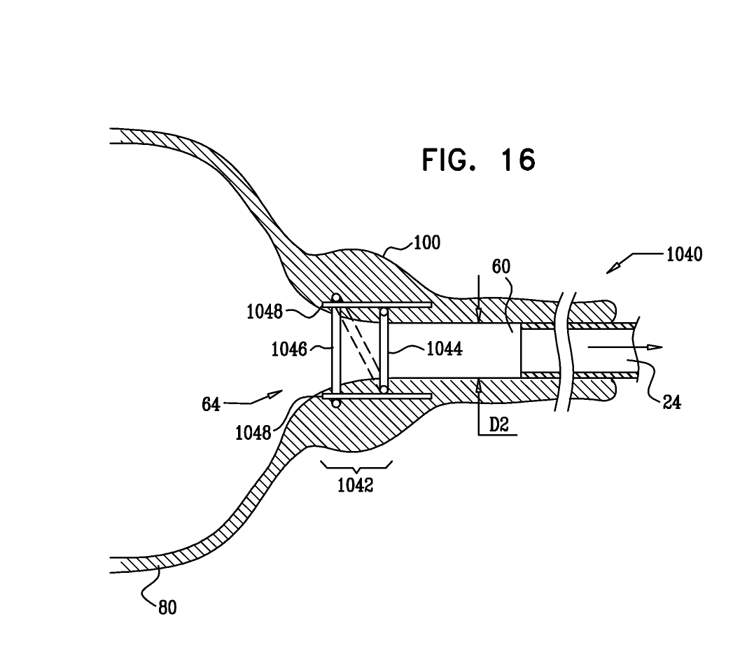

Reference is made to FIGS. 13A-C, which are schematic illustrations of a system 2040 comprising delivery tool 22 coupled to an implant 1200 at a distal end of delivery tool shaft 25, in accordance with an embodiment of the present invention. Urethra 60 is constricted due to pressure exerted thereupon by a prostate 100 of the patient. Implant 1200 comprises a coiled implant comprising a proximal coil 1220 at a proximal end thereof, a distal coil 1260 at a distal end thereof, and a plurality of coils 1201 disposed between coils 1220 and 1260. In some embodiments, implant 1200 comprises a wire 1202 having a circular cross-section (shown in FIG. 13B). In other embodiments, wire 1202 of implant 1200 has a triangular cross-section (shown in FIG. 13C). It is to be noted that wire 1202 may be shaped to define any other suitable shape, e.g., a square, a diamond, or an ellipse, in cross-section thereof. Typically, the shape of wire 1202 helps facilitate pinching of tissue of the patient between the successive coils of implant 1200.

FIG. 13A shows implant 1200 in a resting state thereof in which implant 1200 provides a longitudinal lumen having a diameter larger than a diameter of urethra 60 of the patient. For example, implant 1200 is shaped to define a lumen having a diameter of between 2.5 mm and 15 mm. In its resting state, implant coils 1220 and 1260 each have a respective diameter that is larger than the respective diameters of each of the plurality of coils 1201. In some embodiments, the diameters of coils 1220 and 1260 are substantially equal. Alternatively, the diameter of distal coil 1260 is larger than the diameter of proximal coil 1220.

Typically, implant 1200 has a proximal conic portion 1240 and a distal conic portion 1230. Conic portions 1230 and 1240 have a slope at an angle of between 5-10 degrees, e.g., between 7 and 8 degrees, with respect to the longitudinal axis of tool 22. Conic portion 1240 comprises a plurality of coils that are disposed in series in a manner in which: (1) a proximal-most coil thereof is disposed adjacently to proximal coil 1220, and (2) respective diameters of the coils of portion 1240 decrease in series from (a) the coil adjacent to proximal coil 1220 to (b) a coil of portion 1240 that is furthest from proximal coil 1220. Conically-shaped portion 1230 comprises coils disposed in series in a manner in which: (1) a distal-most coil thereof is disposed adjacently to distal coil 1260, and (2) respective diameters of the coils of the second portion of coils decrease in series from: (a) the distal-most coil of portion 1230 to a proximal-most coil of portion 1230. Such a configuration of implant 1200 helps overcome a force of friction of tissue of the prostate on implant 1200 (in a manner described hereinbelow), as it is implanted around urethra 60.

Typically, prior to introducing delivery tool 22 into urethra 60, outer sheath 24 is advanced distally through proximal end 62 of urethra 60 and toward bladder neck 64 of the patient. Outer sheath 24 expands urethra 60 as sheath 24 is distally advanced toward bladder 80 of the patient. Typically, outer sheath 24 is advanced via an introducer tube (not shown) having a rounded proximal end which facilitates atraumatic advancement of outer sheath 24 through urethra 60. Outer sheath 24 is advanced along urethra 60 prior to the advancement of delivery tool shaft 25, thus creating an open passageway for the subsequent insertion of delivery tool 22. Typically, outer sheath 24 is hollow and enables passage of tools through the urethra by providing a working channel of sheath 24. An imaging device (not shown), e.g., a fiber optic scope or a cystoscope, is advanced through outer sheath 24 into bladder 80. Bladder 80 and bladder neck 64 are examined prior to the introduction of delivery tool 22 into urethra 60 of the patient. The imaging device is typically flexible, and bends 180 degrees in a proximal direction, facilitating visualization of a vicinity of bladder neck 64 of the patient. Alternatively or additionally, an optical sensor, e.g., CCD, CIS, or CMOS, is coupled to a distal portion of shaft 25 of delivery tool 22.

Delivery tool 22 comprises body 21 and delivery tool shaft 25 is advanced distally through outer sheath 24 toward bladder 80 of the patient. Typically, shaft 25 comprises a hollow lumen for passing substances and/or tools therethrough, such as but not limited to, medications, fiber optics, biopsy tools, optical devices (e.g., CCD) and/or other imaging devices.

Typically, implant 1200 comprises a radially-expandable implant, e.g., a coil. Implant 1200 typically comprises a flexible biocompatible material, e.g., nitinol or silicone. During transurethral advancement, implant 1200 is disposed in a compressed state thereof between a proximal implant holder (shown in FIG. 14A as proximal implant holder 124) and distal implant holder 54. In some embodiments, a distal end and a proximal end of implant 1200 are each shaped to define a slit. Each slit is configured for passage of respective fastening devices therethrough. The fastening devices maintain the compressed state of implant 1200 during advancement thereof into bladder 80 of the patient.

Proximal coil 1220 is shaped to define a slit 132 for advancement of a first elongate mechanical fastener 127 therethrough. Fastener 127 functions to hold implant 1200 in place with respect to tool 22 during the advancement of implant 1200 toward prostate 100 of the patient.

Distal implant holder 54 is shaped to provide a groove 56 at a proximal end thereof which holds and secures distal coil 1260 of implant 1200. Together with fastener 127, distal implant holder 54 functions to keep implant 1200 in a compressed state during advancement thereof. Additionally, distal implant holder 54 maintains coupling of implant 1200 to tool 22 during implantation of implant 1200. A second fastener (i.e., similar to fastened 127) fastens distal coil 1260 to distal implant holder 54. The securing and releasing of the fasteners are controlled remotely, by body 21 of delivery tool 22.

The distal-most end of sheath 24 is disposed distally to bladder neck 64, facilitating proper placement within bladder 80 of any device passed through sheath 24, e.g., implant 1200. Typically, outer sheath 24 is shaped to define a length which is shorter than a length of delivery tool shaft 25. Thus, once shaft 25 has been fully advanced through sheath 24, proximal coil 1220 of implant 1200 is disposed distally with respect to the distal-most end of sheath 24. When proximal coil 1220 of implant 1200 has sufficiently entered bladder 80 of the patient (e.g., as shown), proximal end 1220 is released from delivery tool 22, allowing implant 1200 to assume a radially-expanded configuration.

Delivery tool 22 comprises rotating element 30 at a proximal end thereof which is configured to facilitate implantation of implant 1200, once the implant is inside bladder 80 of the patient. During the distal advancing of delivery tool shaft 25 toward bladder 80 of the patient, rotating element 30 is disposed adjacent to body 21 of tool 22, as shown.

Body 21 comprises one or more control elements 28 on a surface of tool 22 which enables a physician to control, from outside of the patient's body, one or more functional elements located at the distal end of delivery tool 22. Typically, but not necessarily, control elements 28 comprise rings for the physician to engage her fingers therethrough and push or pull on control elements 28. During advancement of delivery tool 22 within sheath 24, elements 28 are disposed in a distal orientation with respect to delivery tool 22, e.g., at the distal end of a slot 130 in delivery tool 22 (configuration not shown).

Once the distal end of delivery tool shaft 25 enters bladder 80, implant 1200 is further pushed distally by pushing on switch 42 disposed at the proximal end of tool 22. Such pushing further ensures that proximal coil 1220 of implant 1200 is disposed distal to bladder neck 64 prior to implantation of implant 1200 around urethra 60. Control elements 28 are then pulled proximally with respect to delivery tool 22. Control elements 28 are coupled to a proximal end of fastener 127. In response to the pulling, a distal end of fastener 127 is pulled to a position that is proximal to proximal end 122 of implant 120, thereby releasing proximal coil 1220 of implant 1200 and effecting radial expansion thereof. During radial expansion of implant 1200, distal coil 1260 of implant 1200 remains coupled to distal implant holder 54.

To minimize the chance of physician error, tool 22 may comprise distal lock 32, proximal lock 34, and release 36. Pulling and pushing of control elements 28 is restricted by locks 32 and 34. For example, when elements 28 are disposed distally with respect to tool 22, distal lock 32 automatically maintains the distal position of elements 28 such that elements 28 are not inadvertently pulled (resulting in premature expansion of implant 1200 during advancement thereof). When proximal motion of control elements 28 is desired, the physician activates release 36 to release lock 32, allowing for such proximal motion of elements 28. Once disposed proximally with respect to tool 22, proximal lock 34 secures elements 28 in place, typically automatically.

Distal implant holder 54 is rotatable by switch 42 of tool 22. Implant holder 54 positions implant 1200 coaxially with respect to urethra 60, such that implant 1200 is properly corkscrewed symmetrically around the urethra. An imaging device 70 is then advanced through the lumen of shaft 25, and guides the subsequent implantation of implant 1200. Imaging device 70 is configured to bend 180 degrees and rotate 360 degrees in order to image the implantation procedure.

Delivery tool 22 typically comprises expandable guiding elements 26 which surround a portion of delivery tool shaft 25 proximal to implant 1200. Distal and proximal ends of expandable elements 26 are each coupled to a first ring 27 and a second ring 29, respectively. Typically, first ring 27 is fixed to a portion of shaft 25 while second ring 29 is configured to slide distally and proximally along shaft 25. Alternatively, first ring 27 is configured to slide distally until a stopping element impedes continued distal motion of ring 27. Such distal motion of ring 27 facilitates positioning of ring 27 and distal portions of guiding elements 26 within the lumen of the implant prior to expansion of elements 26. During the advancing of delivery tool shaft 25 toward bladder 80, guiding elements 26 are typically pressed against the outer surface of shaft 25.

Distal pushing of control elements 28 slides ring 29 distally toward ring 27. The distal and proximal ends of expandable elements 26 are drawn toward one another, resulting in the radial expansion of expandable elements 26. Expandable guiding elements 26 expand such that they align with an inner surface of implant 1200. Such alignment facilitates the guiding of implant 1200 and the maintenance of a straight configuration thereof during the implantation procedure.

Typically, once implant 1200 is fully disposed within bladder 80, body 21 of tool 22 is disposed adjacent to a proximal-most end of sheath 24. The implantation of implant 1200 within prostate 100 begins when the physician distances body 21 from outer sheath 24, thereby shifting tool 22 proximally. Such shifting positions proximal coil 1220 of implant 1200 in proximity with bladder neck 64 immediately prior to implantation of implant 1200.

Following expansion within bladder 80 of the patient, implant 1200 is shaped to define an inner lumen diameter, e.g., between 2.5 mm and 15 mm, typically larger than the non-constricted outer diameter of urethra 60. Proximal coil 1220 of implant 1200 comprises pointed tip 122 configured to puncture tissue of prostate 100. In some embodiments, pointed tip 122 is coupled to, e.g., soldered to or attached using any other applicable attachment means, a pointed needle which is configured to puncture tissue of the patient. Typically, the needle of tip 122 comprises a rigid, biocompatible material, e.g., stainless steel, configured to facilitate ongoing penetration of the implant as it is advanced through tissue of prostate 100. It is to be noted that the needle is shaped to define any suitable shape configured for cutting/penetrating tissue.

Following the puncturing of the tissue by pointed tip 122 or, in some embodiments, the needle coupled thereto, implant 1200 is further advanced proximally in the tissue of prostate 100, around urethra 60 of the patient. Counterclockwise rotation of rotating element 30 with respect to a longitudinal axis of tool 22, rotates and proximally retracts implant 1200, thus corkscrewing implant 1200 within tissue of prostate 100 surrounding urethra 60. Positioning of implant 1200 within tissue of prostate 100 is typically guided by imaging element 70.

In some embodiments, as pointed tip 122 of implant 1200 is advanced through the tissue of the patient, it is configured to ablate the tissue. In such an embodiment, implant 1200 may be coated with a substance, such as but not limited to, (a) a medication (e.g., an antibiotic) or (b) an electrical insulator (e.g., Teflon). A proximal portion of implant 1200, i.e., one or more of the coils, may be energized to deliver RF energy, for example, to ablate tissue. In some embodiments, the proximal portion of implant 1200 is coupled to an electrode. Additionally or alternatively, the proximal portion of implant 1200 may be energized to provide ultrasound or thermal energy (e.g., heating or cooling).

In some embodiments, implant 1200 comprises a hollow, helical implant shaped to define a helical lumen and at least one hole, e.g., a plurality of holes, at the proximal end thereof. In such an embodiment, a fluid, e.g., saline, is injected at high pressure through the lumen of the hollow, helical implant and externally to the implant via the at least one hole, in order to cut tissue near the proximal tip of the implant as it advances through the tissue.

In some embodiments, a laser fiber is passed through the lumen of the hollow, helical implant 1200 and through the hole at the proximal end thereof. Typically, the laser fiber ablates tissue in the path of the implant as it is advanced therethrough. In some embodiments, an insulated RF transmitting wire (i.e., having a non-insulated transmitting-tip) is advanced through the helical lumen of the hollow implant.

In some embodiments, a fluid is passed through the lumen of the hollow, helical implant 1200. The hollow, helical implant is shaped to define holes (e.g., typically toward the proximal end of the implant) for release of the fluid externally to the implant. In some embodiments, the fluid comprises a lubricant which passes externally to the implant via the holes defined thereby in order to reduce a frictional force between the tissue and the implant.

Expandable guiding elements 26 guide the initial implantation (e.g., longitudinal motion of 6 mm to 11 mm in a proximal direction) of the proximal portion of implant 1200 around urethra 60. Control elements 28 are then pulled proximally, thereby sliding ring 29 proximally such that guiding elements 26 are pressed once again against the outer surface of shaft 25.

Following initial partial implantation of implant 1200 and alignment of expandable guiding elements 26 along shaft 25, implant 1200 is further advanced proximally through prostate 100, around urethra 60 of the patient. Once implant 1200 is fully implanted in prostate 100, distal coil 1260 is decoupled from distal implant holder 54 by retracting the fastener coupling distal coil 1260 to holder 54.

Typically, as implant 1200 is advanced through tissue of prostate 100, tissue of prostate 100 applies a frictional force to implant 1200. In some embodiments, in order to reduce the effect of the frictional force applied to implant 1200, implant 1200 is coated with a low-friction coating, e.g., PTFE (Teflon), MoST, ADLC or the like. In some embodiments, the implant surface is polished, e.g., electro-polished, mechanically polished, or otherwise, to reduce friction as implant 1200 is advanced through the tissue of the patient.

As proximal coil 1220 of implant 1200 is advanced through the tissue of prostate 100, the tissue applies a frictional force on coils 1201 of coiled implant 1200. In an attempt to continue corkscrewing into the tissue, the tissue exerts an increasingly larger cumulative frictional force on the increasing number of coils that are introduced within prostate 100. In response to the frictional force applied to the intra-prostate coils as they are corkscrewed into the tissue, the coils disposed distally to coil 1220 have a tendency to expand radially, such that the respective diameters of the distal coils are generally similar to the respective diameters of the proximal coils. In such a manner, each successive distal coil of helical implant 1220 enters an opening that is defined by the larger-diameter proximal coil adjacent thereto.

Reference is now made to FIG. 13D, which is a schematic illustration of implant 1200 implanted within prostate 100 of the patient, in accordance with an embodiment of the present invention. Once implant 1200 is implanted within prostate 100, delivery tool shaft 25 and outer sheath 24 are extracted from within urethra 60. Following implantation of implant 1200 within prostate 100, a post-operative diameter of the portion of urethra 60 at prostate 100 is larger than the pre-operative diameter of the portion of urethra 60. Implant 1200 is generally rigid relative to the rigidity of the prostate. The implant thus supports the urethral tissue, minimizing restenosis of urethra 60 should prostate 100 continue to enlarge. Typically, the implant improves urine flow from bladder 80, past bladder neck 64, and through urethra 60. Expanding of the perimeter of urethra 60 typically treats benign prostate hyperplasia.

Following implantation, implant 1200 returns to its resting state thereof, as shown and as described hereinabove with reference to FIG. 13A. Distal coiled portion 1230 increases in diameter from proximal to distal to create improved flow of urine at bladder neck 64 of bladder 80.

Reference is now made to FIGS. 13A-D. It is to be noted that the scope of the present invention includes the implantation of implant 1200 around urethra 60 from within urethra 60, i.e., in a manner in which implant 1200 is not first introduced within bladder 80 prior to implantation of implant 1200 around urethra 60. In such an embodiment, distal coil 1260 comprises a pointed tip, which punctures tissue of prostate 100, and implant 1200 is advanced around the urethra in a proximal-to-distal direction.

Reference is now made to FIGS. 14A-B, which are schematic illustrations of a system 1120 comprising delivery tool 22, a coiled implant 1122 reversibly coupled to tool 22, and a mechanical element 1124 disposed between tool 22 and implant 1122, in accordance with an embodiment of the present invention. Typically, mechanical element 1124 comprises an expandable device, e.g., a balloon, a stent, or a wire basket. In such an embodiment, implant 1122 comprises a substantially rigid material, e.g., stainless steel, by way of illustration and not limitation. For example, implant 1122 may comprise a flexible material such as nitinol. Typically, during advancement of implant 1122 toward prostate 100, implant 1122 is held in a compressed state between distal implant holder 56 and proximal implant holder 124. Proximal implant holder 124 is shaped to provide a groove 125 for holding and securing a proximal end 1121 of implant 1122. Additionally, proximal implant holder 124 is shaped to provide a longitudinal slit for advancement of a first elongate mechanical fastener 127 therethrough and subsequently through slit 132 of proximal end 1121 of implant 1122. Fastener 127 is advanced (a) through the longitudinal slit within holder 124, (b) subsequently through slit 132 of proximal end 1121 of implant 1122, and (c) back into a slit at a portion of holder 124 distal to groove 125.

Reference is now made to FIG. 14B. Once implant 1122 is disposed within bladder 80 and the proximal end of implant 1122 is released from proximal implant holder 124, mechanical element 1124 expands within the lumen defined by implant 1122 and forces the surrounding implant 1122 to expand in turn. In such an embodiment, implant 1122 is expanded to a desired diameter, e.g., between 2.5 mm and 15 mm, that is suitable to facilitate implantation of implant 1122 around urethra 60.

In some embodiments, implant 1122 is expanded within urethra 60 and is not first advanced into bladder 80. In such an embodiment, implant 1122 is implanted around urethra 60 from within urethra 60 in a proximal-to-distal direction. Alternatively, implant 1122 is expanded such that it exerts a force on an inner wall of urethra 60 and is resorbed by the urethra without puncturing urethra 60.

It is to be noted that the scope of the present invention includes the expanding of implant 1122 independently of mechanical element 1124. In such an embodiment, in order to expand implant 1122, a first end of implant 1122 is held in place while a second end of implant 1122 is twisted to expand coils of implant 1122. For example, a proximal end of implant 1122 may be held in place by proximal implant holder 124 while a distal end of implant 1122 is rotated by rotating distal implant holder 54 with respect to shaft 25.

FIG. 15 shows a system 1030 comprising delivery tool 22 coupled to a motor 1032, in accordance with an embodiment of the present invention. Typically, motor 1032 helps facilitate implantation of implant 120 around urethra 60 of the patient. In some embodiments, motor 1032 controls the corkscrewing of implant 120 into tissue of prostate 100 by imparting a jackhammer-like function, or force, to the implant. Motor 1032 causes implant 120 to move in fast jerks when entering tissue so as to overcome the force of friction as it is advanced into the tissue. As implant 120 is functioning as a jackhammer, the physician also rotates the handle (in the direction as indicated by the arrow) to facilitate the corkscrewing of the implant into the tissue. In some embodiments, a second motor may be coupled to tool 22 which is used to rotate implant 120 in order to facilitate corkscrewing of implant 120 around urethra 60.

In some embodiments, motor 1032 is configured to facilitate oscillation of implant 120 as it is advanced within tissue of the patient. In such an embodiment, motor 120 causes implant to be rotationally advanced and retracted by given rotational distances. Typically, motor 1032 cycles between facilitating (a) advancement of implant 120 into the tissue by a first number of degrees, and (b) retraction of implant 120 by a second number of degrees. For example, motor 1032 may cause implant to be rotated 270 degrees in order to be implanted into the tissue, and subsequently, motor 1032 may cause implant to be retracted by 60 degrees. Such oscillation of implant between implanting and retracting helps overcome the friction that the tissue of prostate 100 applies to implant 120.

In some embodiments, motor 1032 comprises a vibrator configured to vibrate, which causes implant 120 to agitate the tissue of prostate 100 as implant 120 is implanted therein. In some embodiments, motor 1032 comprises a source of ultrasound energy, e.g., an ultrasound transducer, which causes implant 1032 to vibrate in response to ultrasound energy created by the transducer. Such vibration helps overcome the friction applied to implant 120 as it is implanted in the tissue of prostate 100.

It is to be noted that motor 1032 is coupled to tool 22 at body 21 thereof by way of illustration and not limitation, and that motor 1032 may be coupled to any portion of delivery tool 22. In some embodiments, motor 1032 may be coupled to implant 120. For embodiments in which motor 1032 is coupled to a portion of tool 22 that is remote from body 21, motor 1032 may be remotely controllable. In some embodiments, motor 1032 is used to automate rotation of implant 120 in order to facilitate automated corkscrewing of implant 120.

It is to be noted that implant 120 is shown in FIG. 15 by way of illustration and not limitation, and that delivery tool 22 coupled to motor 1032 may be used to implant any one of implants described herein.

Reference is now made to FIG. 16, which is a schematic illustration of a system 1040 comprising a coiled implant 1042 having a proximal coil 1044 and a distal coil 1046, which form a scaffold for supporting a plurality of longitudinal implant rods 1048, in accordance with an embodiment of the present invention. Typically implant 1042 and rods 1048 comprise a biocompatible material, e.g., nitinol, silicone, and/or stainless steel. Typically, implant 1042 is shaped to provide between 1.5 and 2 coils, which define a conically-shaped implant. Proximal coil 1044 has a diameter that is smaller than a diameter of distal coil 1046. The respective diameters of coils 1044 and 1046 are each larger than a diameter of urethra 60, i.e., each coil 1044 and 1046 has a diameter of between 2.5 mm and 15 mm.

Implant 1042 is implanted around urethra 60 by being corkscrewed therearound. In some embodiments, implant 1042 is first advanced into bladder 80 and is corkscrewed proximally around urethra 60. Alternatively, implant 1042 is corkscrewed, e.g., distally around urethra 60, from within urethra 60.

In either embodiment, rigid longitudinal rods 1048 are implanted in urethra 60 and are supported therein by implant 1042, which functions as a scaffold. Each rod 1048 is first advanced into bladder 80. Once inside bladder 80, rod 1048 is then retracted proximally into tissue of prostate 100. Rod 1048 is advanced with respect to implant 1042 in a manner in which rod 1048 is advanced below the inner surface of distal coil 1046 and above an outer surface of proximal coil 1044. Typically, the plurality of rods 1048 are implanted substantially in parallel with urethra 60 of the patient.

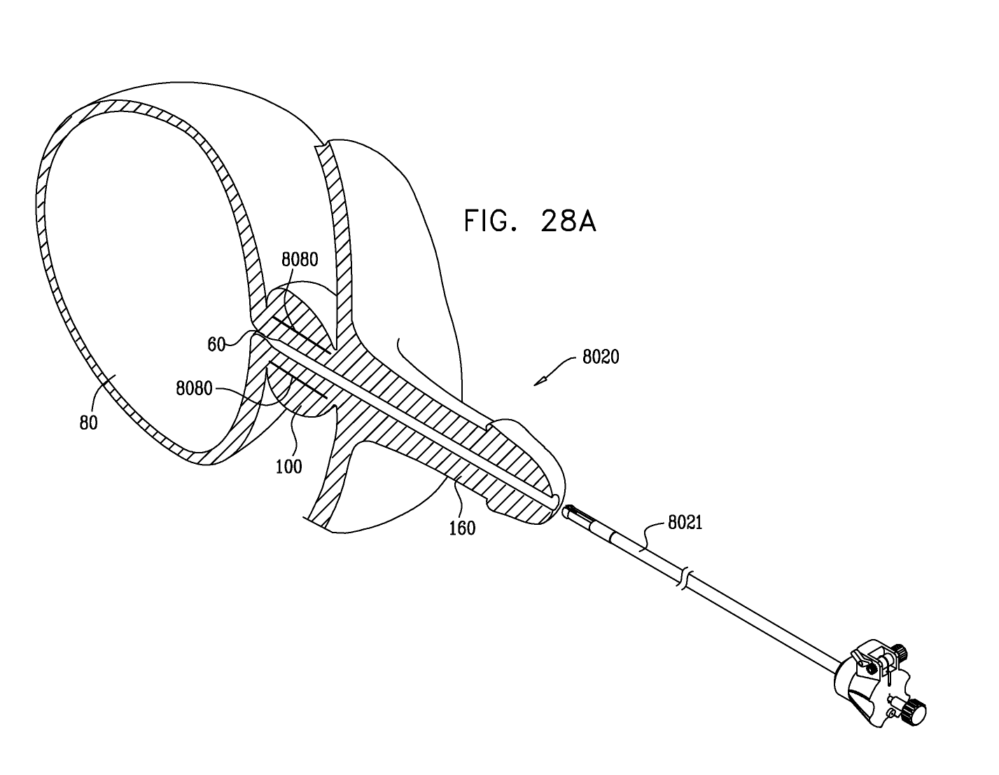

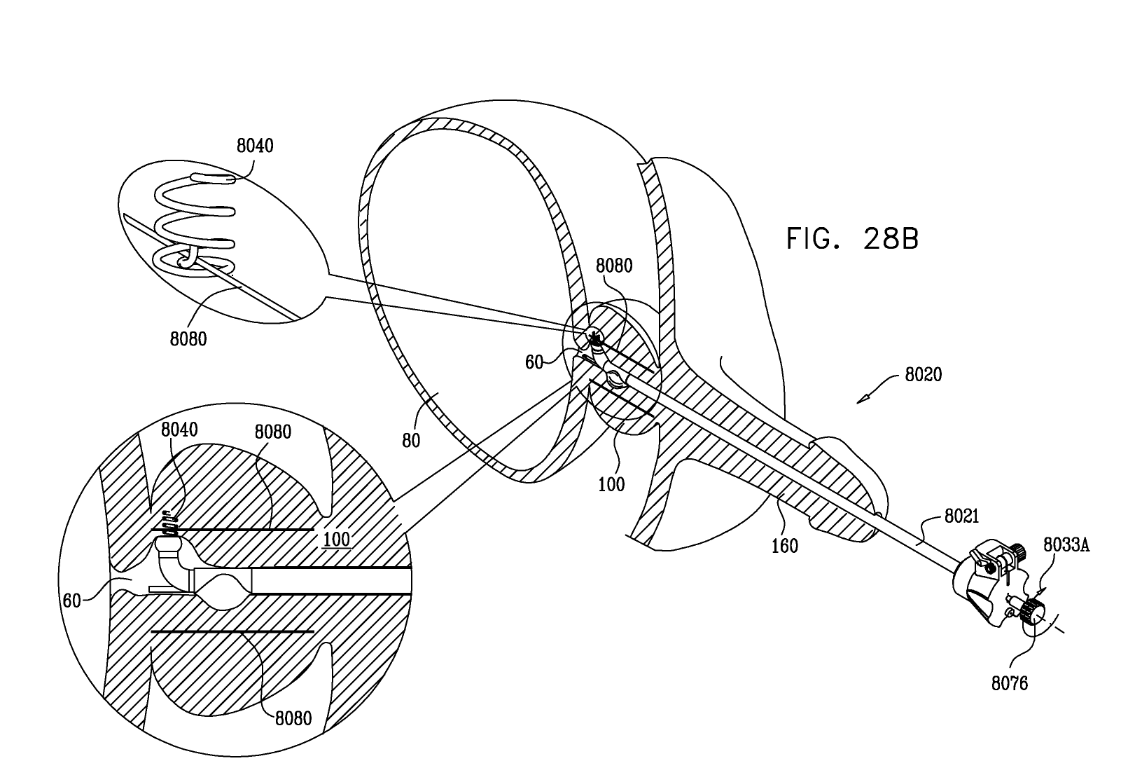

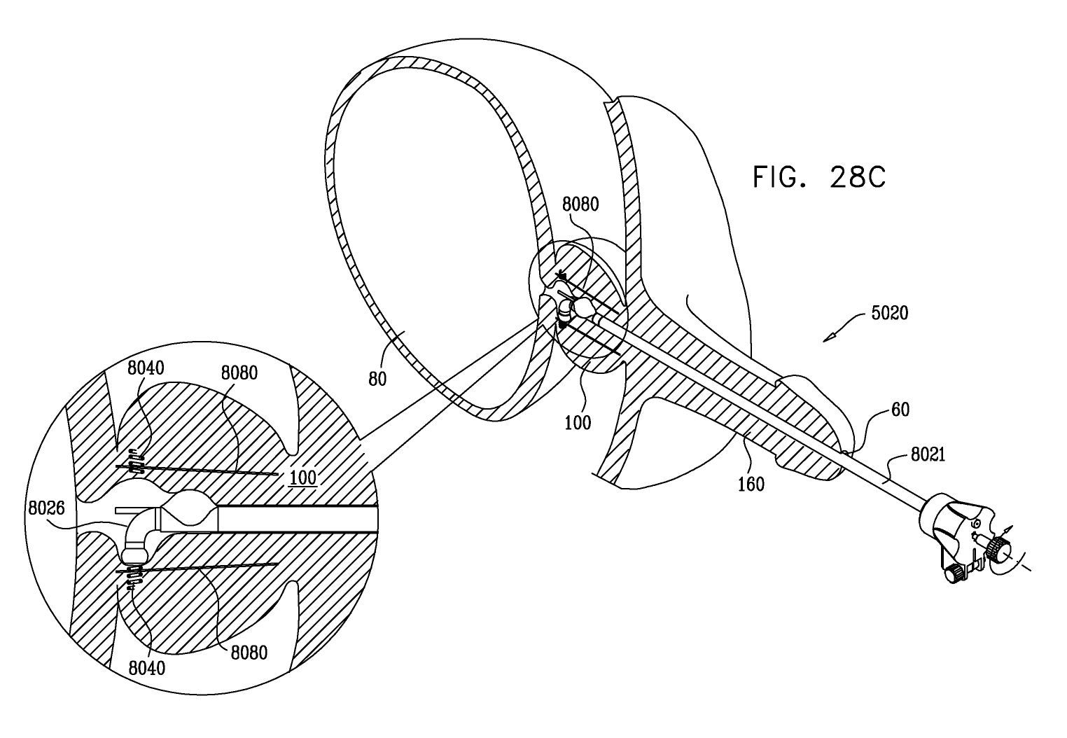

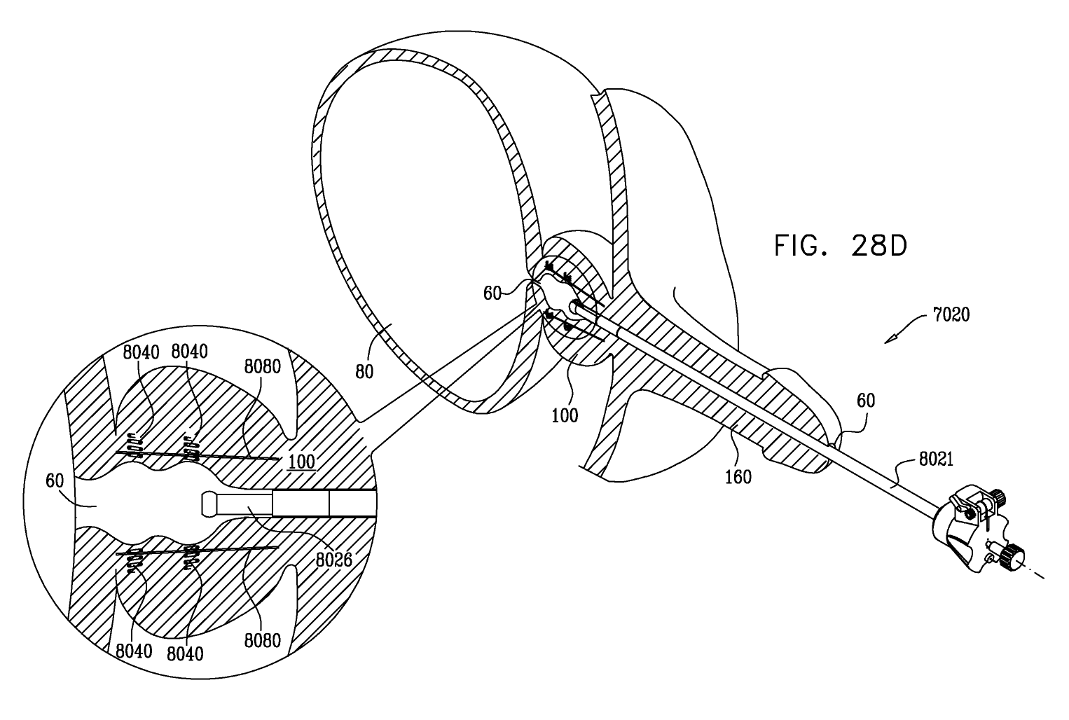

It is to be noted that for some embodiments of the present invention, rods 1048 may be implanted prior to implantation of implant 1042. In such an embodiment, rods 1048 function to support implant 1042 (as described hereinbelow with reference to FIGS. 28A-D).

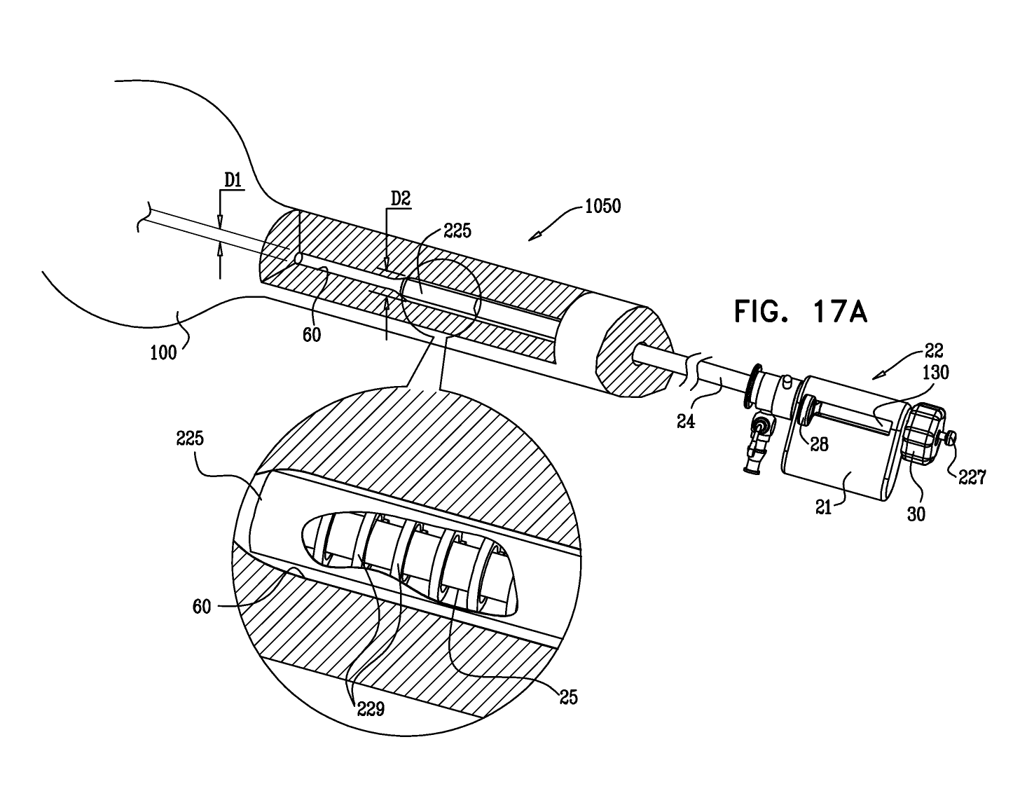

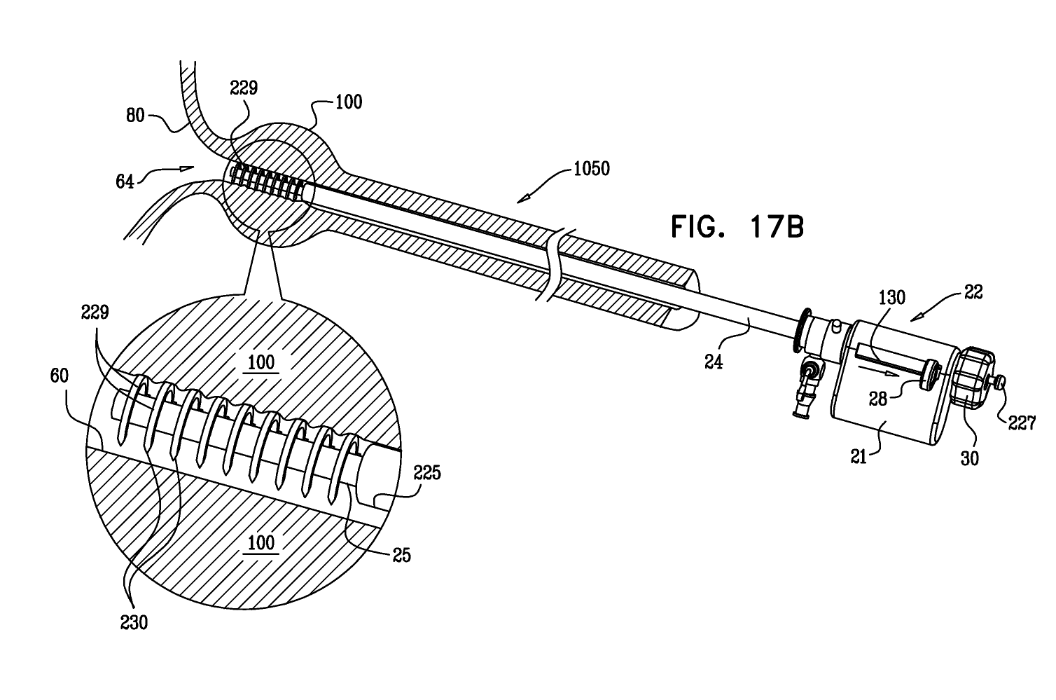

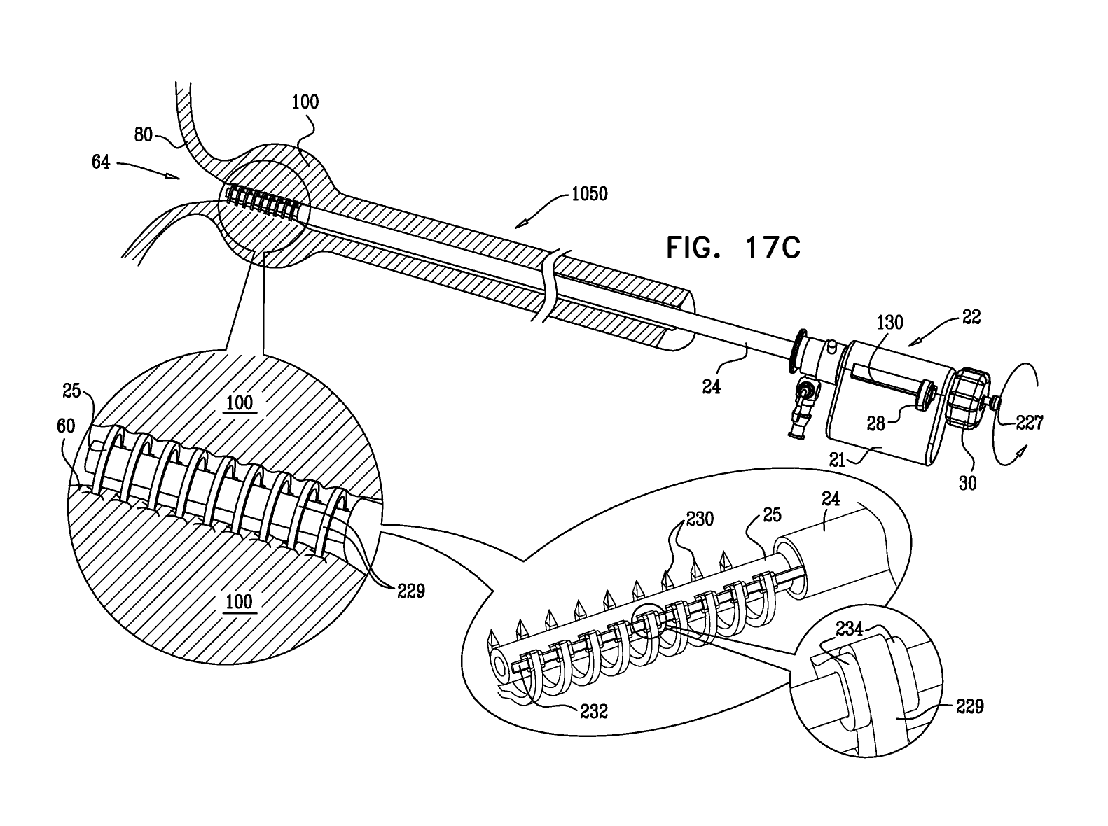

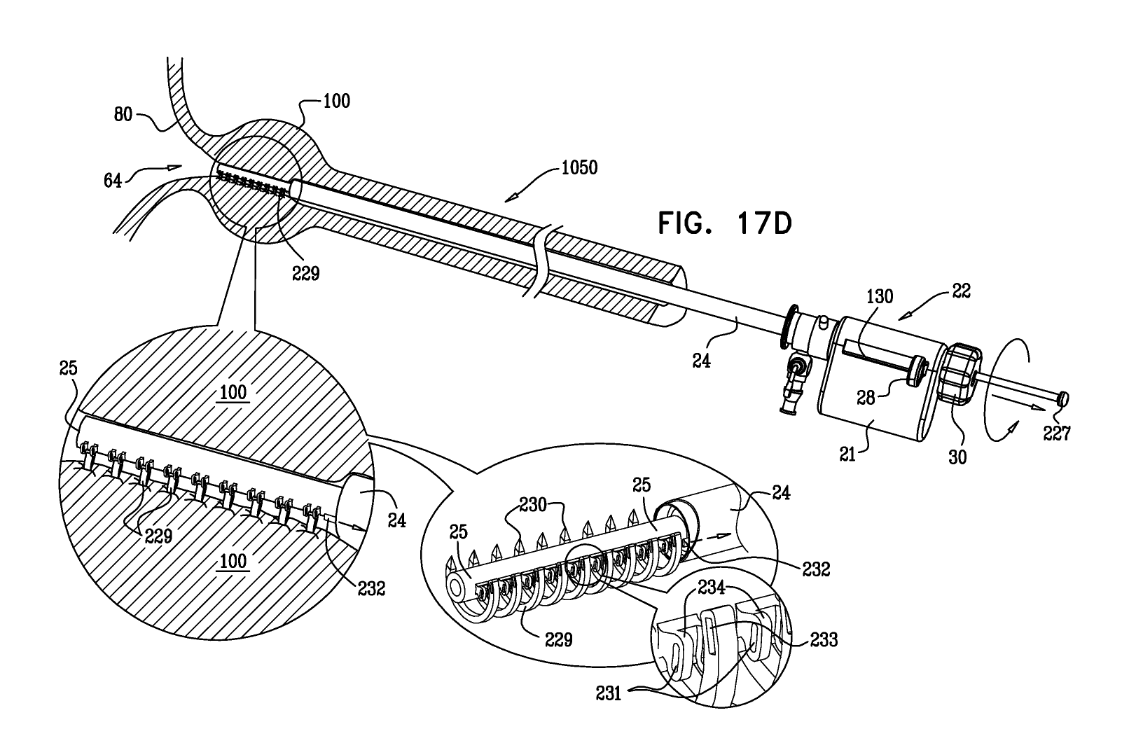

FIGS. 17A-D show a system 1050 comprising delivery tool 22 reversibly coupled to and facilitating implantation around urethra 60 of a plurality of curved needles 229, in accordance with an embodiment of the present invention. Typically, needles 229 comprise an expandable material, e.g., nitinol. During advancement of needles 229 toward prostate 100, needles 229 are compressed between a distal portion of delivery tool shaft 25 and a retractable sheath 225 (FIG. 17A). When compressed within sheath 225, needles 229 are tightly wrapped around shaft 25. Prior to the advancement of needles 229 through urethra 60, urethra 60 defines a constricted, preoperative diameter D1 at prostate 100. As shaft 25 is advanced through urethra 60, a distal portion of shaft 25, needles 229, and sheath 225 expand urethra 60 to a diameter D2. Following implantation of needles 229 around urethra 60, needles 229 maintain a postoperative diameter D2 of urethra 60 at prostate 100.

Reference is now made to FIG. 17B. Body 21 of delivery tool 22 is shaped to define one or more slots 130 which facilitate back and forth sliding of mechanical control elements 28. Control elements 28 are pulled proximally and, responsively, control the retraction of sheath 225 proximally with respect to needles 229. Following the retraction of sheath 225, needles 229 are exposed within urethra 60 at prostate 100. Once exposed, needles 229 expand and push against tissue of prostate 100 that constricts urethra 60. Needles 229 typically expand such that an inner lumen defined by each needle 229 is larger than a diameter of urethra 60 at prostate 100. For example, the inner lumen of each needle 229 has a diameter, e.g., between 2.5 mm and 15 mm, that is larger than diameter D2 (diameter D2 shown in FIG. 17A).

Typically, needles 229 comprise curved needles which are each shaped to define between 180 and 360 degrees, e.g., between 250-300 degrees in a resting state thereof (shown in FIGS. 17B-D). Each needle 229 is shaped to provide a pointed tip 230. In some embodiments, tip 230 comprises stainless steel which is welded to the body of needle 229.

FIG. 17C shows partial implantation of needles 229 in tissue of prostate 100 that surrounds urethra 60. Rotating element 30 is rotated in a counter-clockwise direction, i.e., in the direction such that the pointed ends of each needle 229 enter tissue of prostate 100.

Needles 229 are coupled together during the advancement toward prostate 100 and subsequent implantation around urethra 60 (FIG. 17C). Following implantation of needles 229 around urethra 60 (as shown in FIG. 17D), needles 229 are decoupled from one another.

Reference is now made to FIGS. 17C-D. Needles 229 are coupled together by a longitudinal bar 232 which is configured for slidable advancement with respect to needles 229. As shown in FIG. 17D, each needle 229 (at an end thereof that opposes pointed tip 230) is shaped to define a longitudinal slit 233 for passage therethrough of bar 232. Each needle 229 is coupled to a respective needle holder 234 that is coupled to delivery tool shaft 25. Needle holder 234 has a pair of arms which surround the end of needle 229. The arms of needle holder 234 are each shaped to provide a longitudinal slit 231 that is in alignment with slit 233 of needle 229 when the end of needle 229 is disposed within holder 234. When needles 229 are coupled together, for each needle 229 and needle holder 234, bar 232 passes through slit 231 of a first arm of needle holder 234, through slit 233 of needle 229, and finally through slit 231 of a second arm of needle holder 234.

FIG. 17D shows the decoupling of bar 232 from needles 229 following implantation of needles 229 around urethra 60. Bar 232 is controlled by a mechanical element 227. By pulling on mechanical element 227, in a direction as indicated by the arrow, bar 232 is retracted into sheath 24 and releases needles 229. Typically, the ends of needles 229 that oppose pointed tips 230 remain disposed within urethra 60 following the initial implantation of needles 229. Ultimately, the ends are resorbed into the tissue surrounding urethra 60.

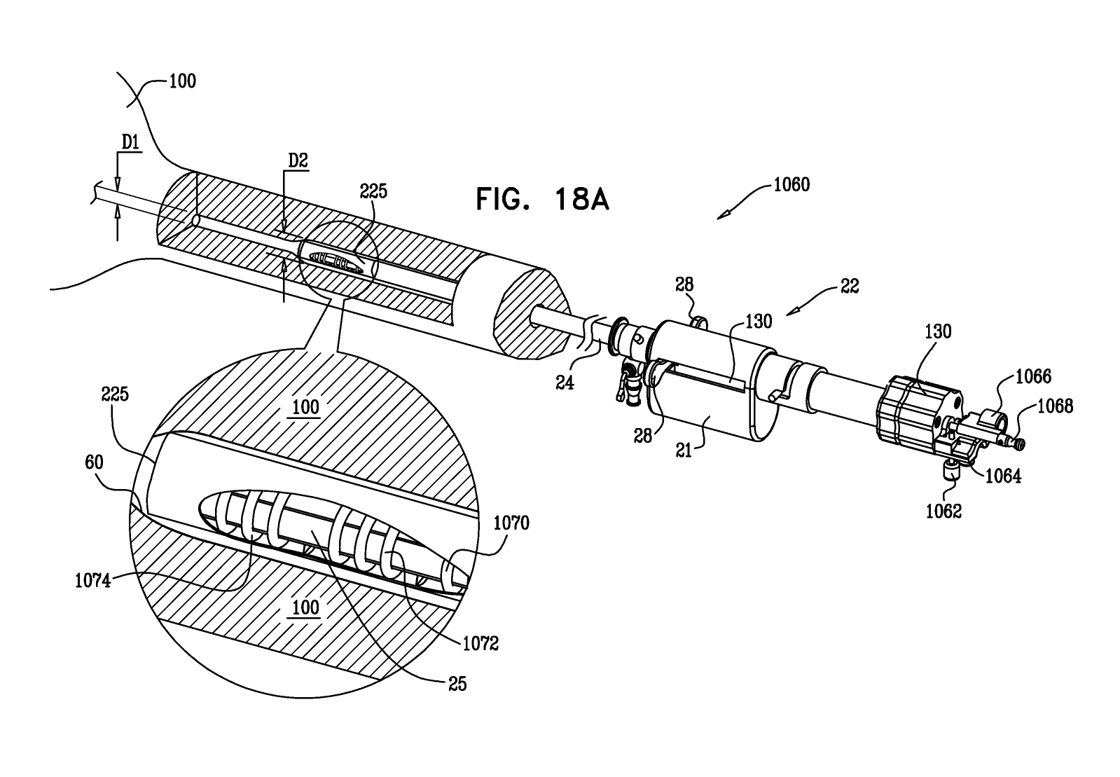

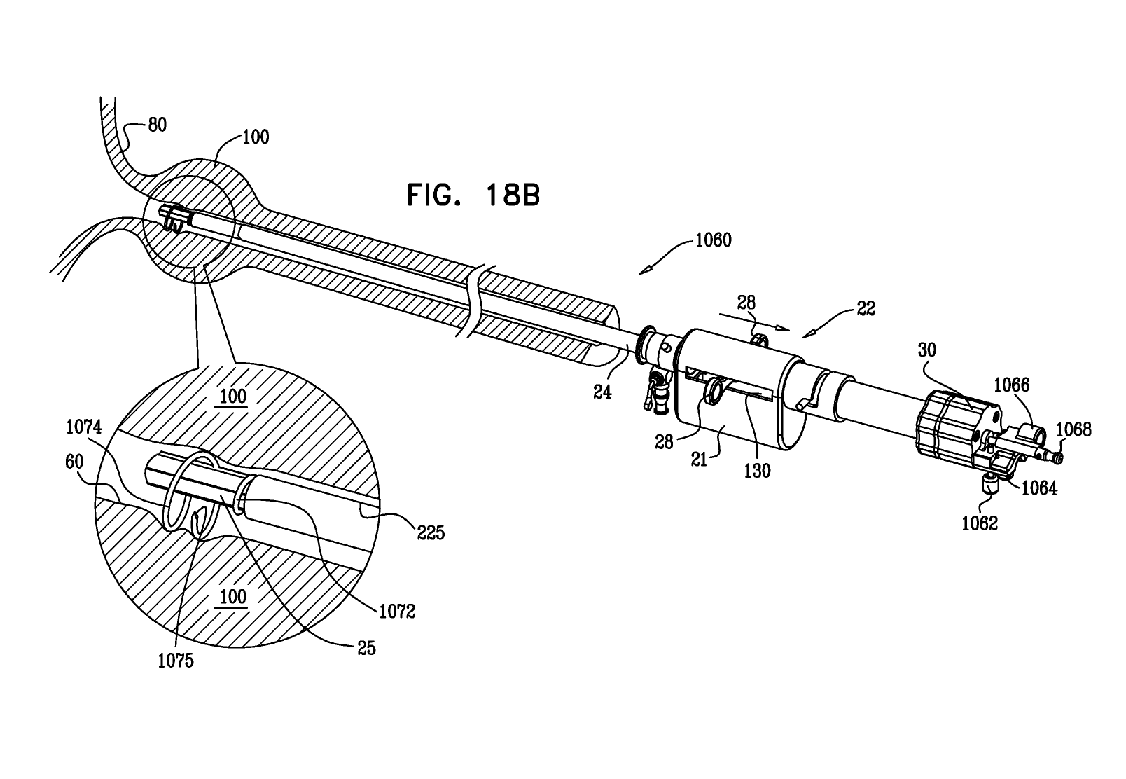

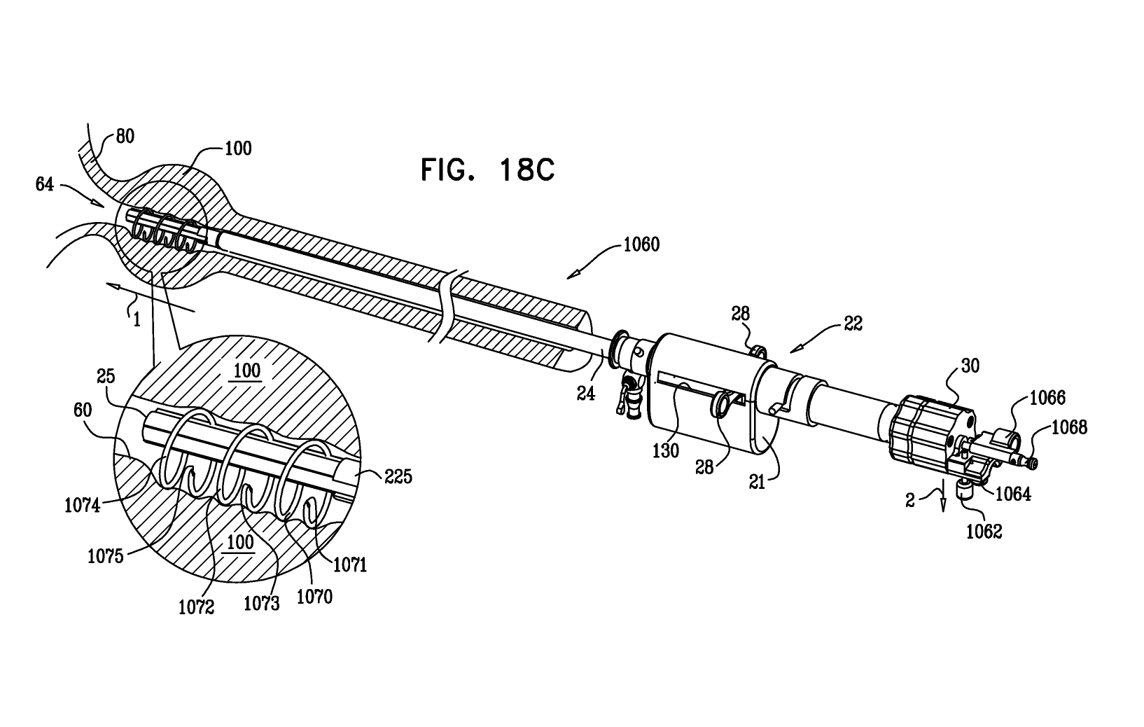

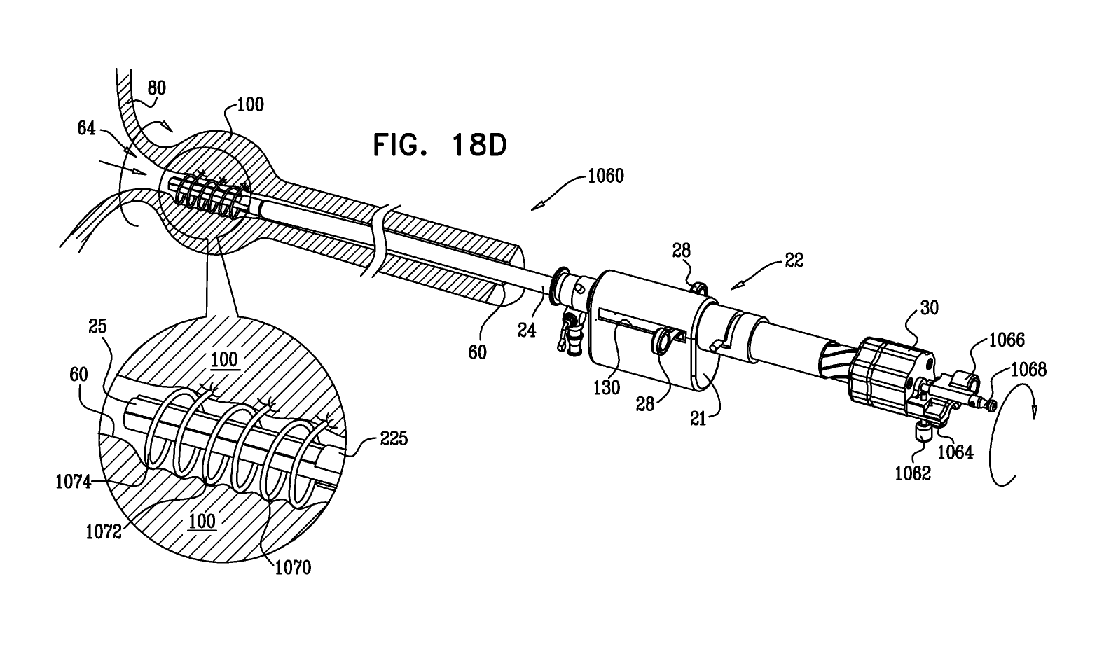

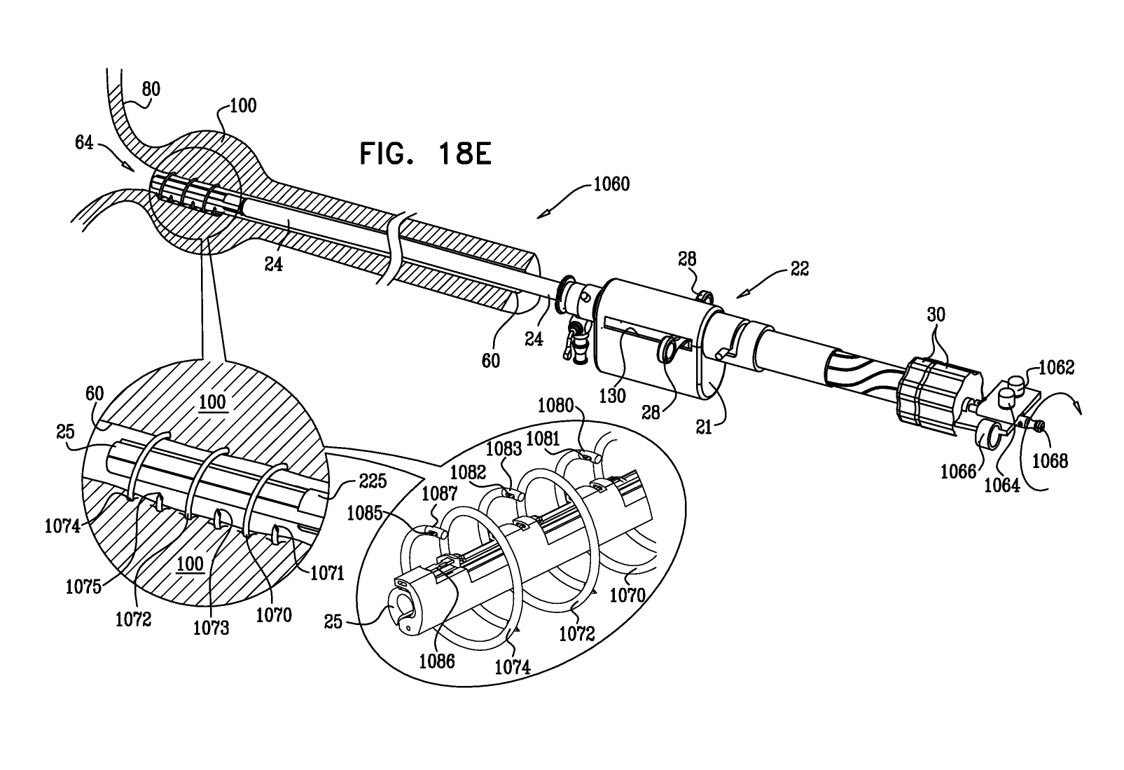

Reference is now made to FIGS. 18A-E, which are schematic illustrations of a system 1060 comprising delivery tool 22 reversibly coupled to and facilitating implantation around urethra 60 of a plurality of coiled implants 1070, 1072, and 1074, in accordance with an embodiment of the present invention. Typically, coiled implants 1070, 1072, and 1074 comprise a flexible material, e.g., nitinol. During advancement of needles 229 toward prostate 100, implants 1070, 1072, and 1074 are compressed between a distal portion of delivery tool shaft 25 and retractable sheath 225 (FIG. 18A). Implants 1070, 1072, and 1074 are typically wrapped tightly around shaft 25 when compressed within sheath 225.

Prior to the advancement of implants 1070, 1072, and 1074 through urethra 60, urethra 60 defines a constricted, preoperative diameter D1 at prostate 100. As shaft 25 is advanced through urethra 60, a distal portion of shaft 25, implants 1070, 1072, and 1074, and sheath 225 expand urethra 60 to a diameter D2. Following implantation of implants 1070, 1072, and 1074 around urethra 60, implants 1070, 1072, and 1074 will maintain a postoperative diameter D2 of urethra 60 at prostate 100.

Implants 1070, 1072, and 1074 are advanced through urethra 60 until they are disposed in urethra 60 in the vicinity of prostate 100. FIG. 18B shows partial retraction of sheath 225 by pulling on mechanical controls 28 along slots 130 in the direction as indicated by the arrow. As sheath 225 is retracted and implants 1070, 1072, and 1074 are exposed, implants 1070, 1072, and 1074 expand and push against tissue of prostate 100 that constricts urethra 60.

FIG. 18C shows complete retraction of sheath 225 and expansion of implants 1070, 1072, and 1074. Once exposed and expanded, implants 1070, 1072, and 1074 push against tissue of prostate 100 that constricts urethra 60. Each implant 1070, 1072, and 1074 is shaped to provide a respective pointed distal tip 1071, 1073, and 1075. In some embodiments, tips 1071, 1073, and 1075 comprise stainless steel tips which are welded to implants 1070, 1072, and 1074, respectively.

Reference is now made to FIGS. 18A and 18C. In a compressed state during delivery of implants 1070, 1072, and 1074 through urethra 60, implants 1070, 1072, and 1074 are tightly wound around shaft 25 of delivery tool 22, and have 2-5 coils (e.g., 3-4 coils as shown in FIG. 6A). Following expansion of implants 1070, 1072, and 1074, implants 1070, 1072, and 1074 are in their relaxed states in which implants 1070, 1072, and 1074 have 1-5 coils (typically, 2-3 coils, as shown). Implants 1070, 1072, and 1074 typically expand such that an inner lumen defined by each implant 1070, 1072, and 1074 is larger than a diameter of urethra 60 at prostate 100. For example, the inner lumen of each implant 1070, 1072, and 1074 has a diameter, e.g., between 2.5 mm and 15 mm, which is larger than diameter D2 (diameter D2 shown in FIG. 18A).

During delivery of implants 1070, 1072, and 1074 through urethra 60, implants 1070, 1072, and 1074 are coupled together by a longitudinal bar, as described hereinbelow.

Reference is again made to FIG. 18C. Delivery tool 22 comprises mechanical locks 1062 and 1064 which allow for certain mechanical activity of delivery tool 22 only when locks 1062 and 1064 are released. For example, lock 1062 is released by pulling downward on a knob of lock 1062 in a direction as indicated by the arrow. Releasing lock 1062 allows for the operating physician to distally advance implants 1070, 1072, and 1074 slightly within urethra 60, without having to distally push the entire delivery tool 22.

FIG. 18D shows partial implantation of implants 1070, 1072, and 1074 in response to counterclockwise rotation of rotating element 30 (i.e., in the direction such that the pointed ends of each implant 1070, 1072, and 1074 enter tissue of prostate 100). Rotation of rotating element 30 also proximally distances element 30 from body 21 which retracts implants 1070, 1072, and 1074 as they are corkscrewed around urethra 60.

FIG. 18E shows implants 1070, 1072, and 1074 in their fully-implanted state around urethra 60. Implants 1070, 1072, and 1074 maintain an unconstricted state of urethra 60 at prostate 100. Following implantation, implants 1070, 1072, and 1074 are decoupled from one another.

Typically, implants 1070, 1072, and 1074 are coupled together by a longitudinal bar 1086 which is configured for slidable advancement with respect to implants 1070, 1072, and 1074. Each implant 1070, 1072, and 1074, at respective ends 1080, 1083, and 1087 thereof (i.e., at an end thereof that opposes pointed tips 1071, 1073, and 1075, respectively) is shaped to define a respective groove 1081, 1082, and 1085 for passage therethrough of bar 1086. Typically, grooves 1081, 1082, and 1085 are shaped to define “T”-shaped grooves which surround respective portions of bar 1086. The portions of bar 1086 that are disposed within grooves 1081, 1082, and 1085 are shaped to define narrow portions which are configured to be slid within and displaced from within grooves 1081, 1082, and 1085 in response to a force applied thereto.

FIG. 18E shows the decoupling of bar 1086 from implants 1070, 1072, and 1074 following implantation thereof around urethra 60. Bar 1086 is controlled by a mechanical element 1068. By pulling on mechanical element 1068, bar 1086 is agitated and retracted slightly such that the portions of bar 1086 that are disposed within grooves 1081, 1082, and 1085 move out of grooves 1081, 1082, and 1085, thereby releasing implants 1070, 1072, and 1074. Typically, ends 1080, 1083, and 1087 of implants 1070, 1072, and 1074, respectively, remain disposed within urethra 60 following initial implantation of the implants. Ultimately, ends 1080, 1083, and 1087 are resorbed into the tissue surrounding urethra 60.

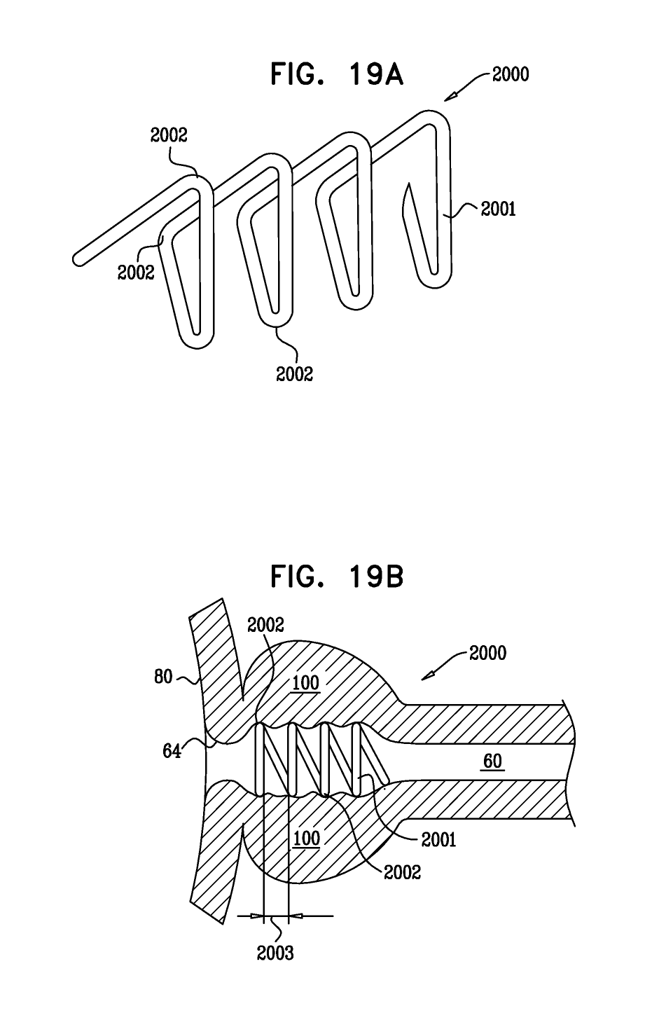

Reference is now made to FIGS. 19A-B which are schematic illustrations of an implant 2000 shaped to define vertices 2002, in accordance with an embodiment of the present invention. Implant 2000 is typically resorbable. Typically, implant 2000 is shaped to define a prism having a triangular face when viewed in cross-section. In some embodiments, implant 2000 comprises a radially-expandable implant comprising a flexible material, e.g., nitinol. In some embodiments, implant 2000 is less flexible, e.g., comprising stainless steel, which is nevertheless expandable by a mechanical element (as described hereinabove with reference to FIGS. 14A-B).

As shown in FIG. 19B, implant 2000 is delivered to a portion of urethra 60 that is in the vicinity of prostate 100. Implant 2000 is either (a) allowed to expand (in embodiments in which implant 2000 comprises an expandable material such as nitinol) or (b) is made to expand using a mechanical element, such that vertices 2002 are in contact with an inner surface of urethra 60. A respective area 2003 is defined between neighboring vertices 2002 of implant 2000. Typically, tissue of urethra 60 is pinched into areas 2003 between neighboring vertices 2002. Ultimately, implant 2000 is resorbed by urethra 60 and into tissue of prostate 100.

In some embodiments, implant 2000 is coated with a pro-fibrotic agent which helps enhance the resorption of implant 2000 into prostate 100.

In some embodiments, implant 2000 comprises a wire 2001 having a circular cross-section. In other embodiments, wire 2001 of implant 2000 has a triangular cross-section. It is to be noted that wire 2001 may be shaped to define any other suitable shape, e.g., a square or an ellipse, in cross-section thereof. Typically, the shape of wire 2001 helps facilitate pinching of tissue of the patient between the successive coils of implant 2000.

It is to be noted that implant 2000 is shaped to define a prism by way of illustration and not limitation. For example, implant 2000 may be shaped to define a cylinder having a circular or elliptical face when viewed in cross-section. In other embodiments, implant 2000 may be shaped to define a rectangle having a square or diamond-shaped face when viewed in cross-section.

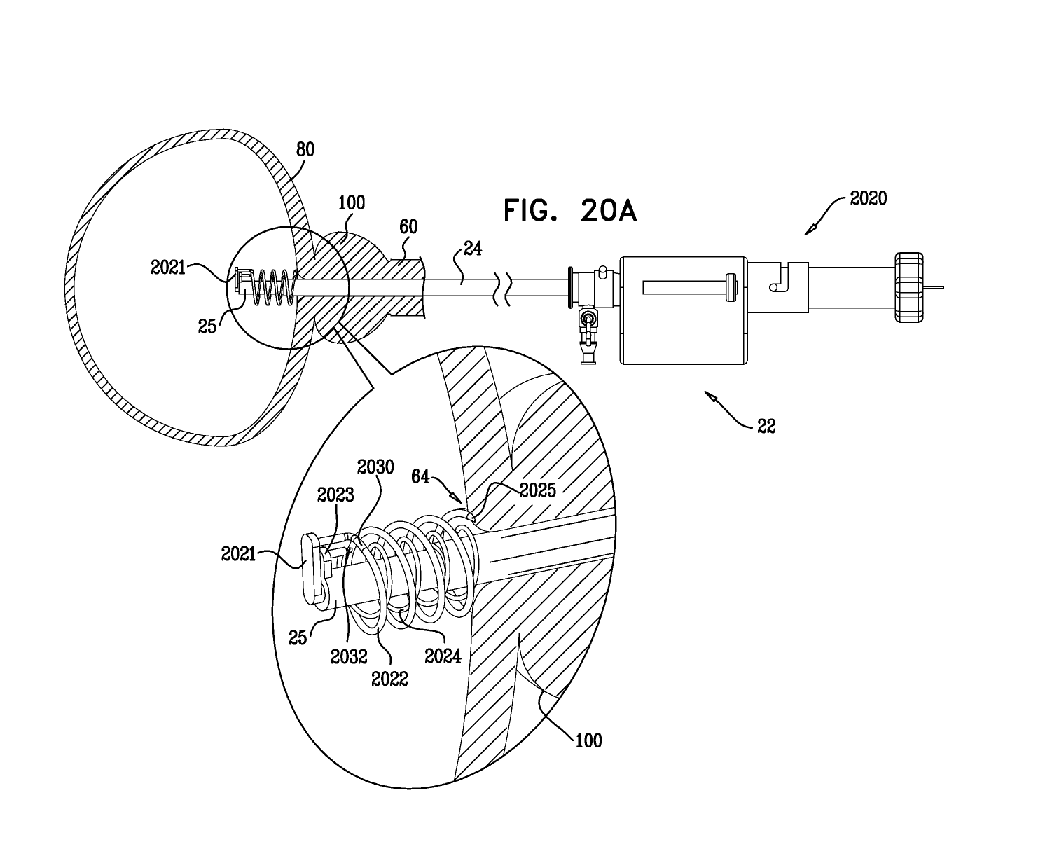

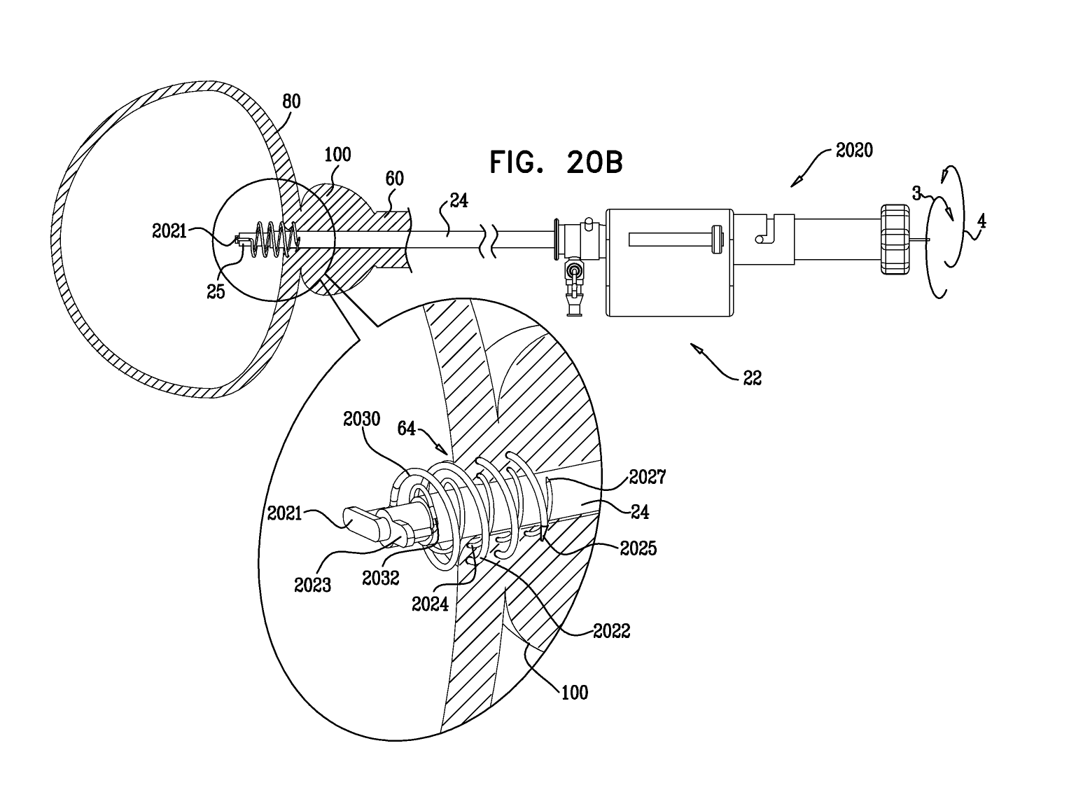

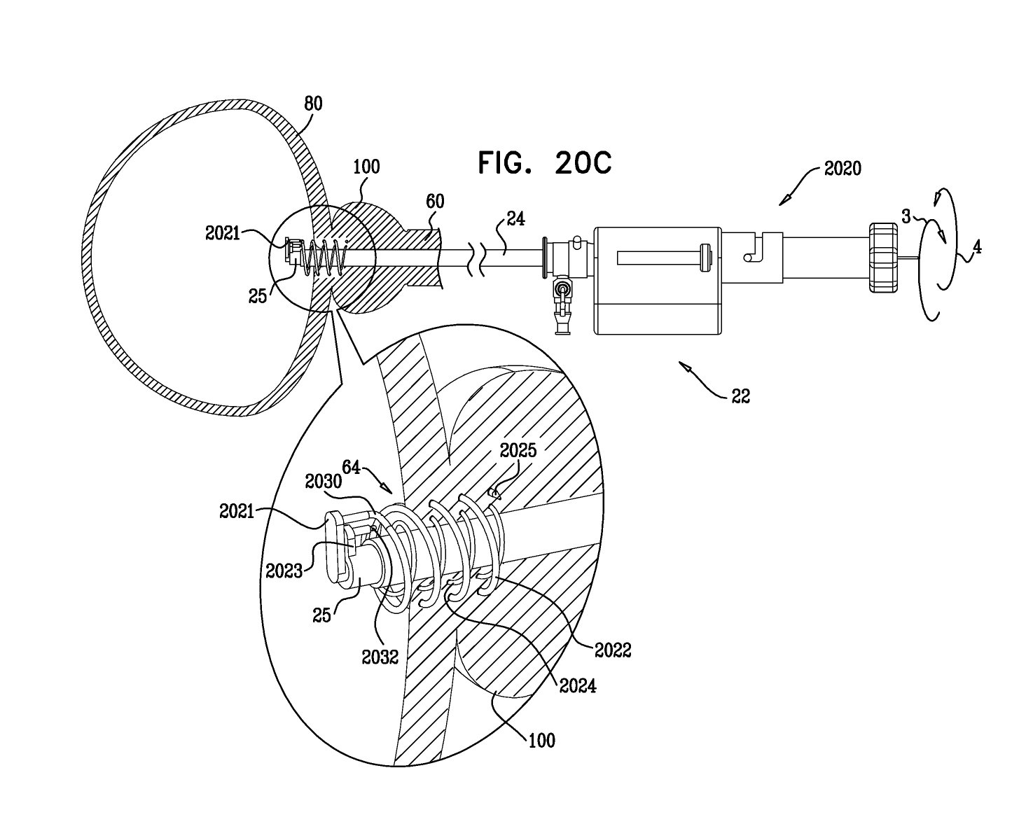

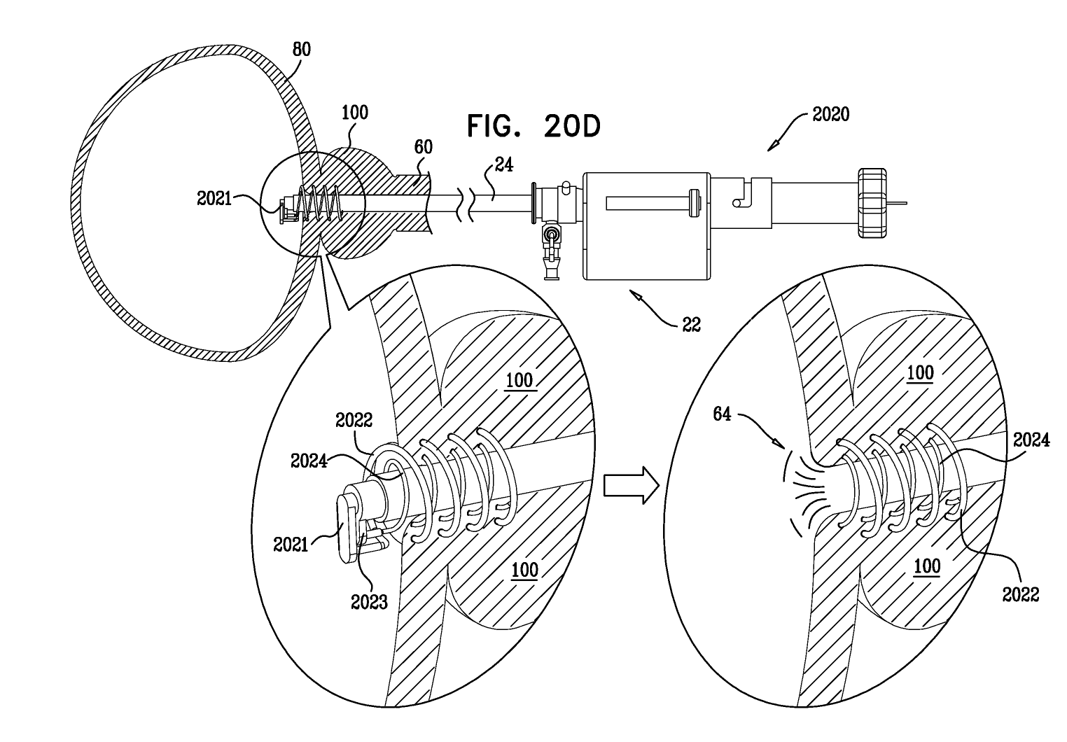

FIGS. 20A-D show a system, 2020 comprising delivery tool 22 reversibly coupled to and facilitating implantation around urethra 60 of a plurality of a first coiled implant 2022 and a second coiled implant 2024, in accordance with an embodiment of the present invention. Implant 2022 is shaped to define a left-handed coil, and implant 2024 is shaped to define a right-handed coil. Implant 2024 has an outer diameter that is smaller than an inner diameter of implant 2022. Implant 2022 is shaped from a wire having a width that is larger than the width of the wire used to shape implant 2024. Implants 2022 and 2024 are each shaped to provide an inner lumen which has a diameter that is larger than a diameter of urethra 60. Typically, implants 2022 and 2024 are coupled to delivery tool 22 in a relative spatial configuration in which implant 2024 is disposed concentrically within implant 2022.

It is to be noted that although two implants 2022 and 2024 are shown, any suitable number of implants may be reversibly coupled to delivery tool 22. In some embodiments, a respective portion of implant 2022 and 2024 ablates tissue of the patient as it is advanced therethrough. In such an embodiment, implants 2022 and 2024 may be coated with a substance, such as but not limited to, (a) a medication (e.g., an antibiotic) or (b) an electrical insulator (e.g., Teflon). One or more of the coils of implants 2022 and 2024, may be energized to deliver RF energy, for example, to ablate tissue. In some embodiments, a respective portion of implants 2022 and 2024 is coupled to an electrode. Additionally or alternatively, a respective portion of implants 2022 and 2024 may be energized to provide ultrasound or thermal energy (e.g., heating or cooling).

In some embodiments, implants 2022 and 2024 each comprise a hollow, helical implant shaped to define a helical lumen and at least one hole, e.g., a plurality of holes, at the proximal end thereof. In such an embodiment, a fluid, e.g., saline, is injected at high pressure through the respective lumens of the hollow, helical implants and externally to the implants via the at least one hole, in order to cut tissue near the puncturing tip of the implant as it advances through the tissue.

In some embodiments, a respective laser fiber is passed through the lumen of the each one of hollow, helical implants 2022 and 2024 and through the hole at the proximal end thereof. Typically, the laser fiber ablates tissue in the path of the implant as it is advanced therethrough. In some embodiments, an insulated RF transmitting wire (i.e., having a non-insulated transmitting-tip) is advanced through the helical lumen of the hollow implant.

In some embodiments, a fluid is passed through each one of the lumens of the hollow, helical implants 2022 and 2024. Each hollow, helical implant is shaped to define holes (e.g., typically toward the proximal end of the implant) for release of the fluid externally to the implant. In some embodiments, the fluid comprises a lubricant which passes externally to the implant via the holes defined thereby in order to reduce a frictional force between the tissue and the implant.

Typically, as implants 2022 and 2024 are advanced through tissue of prostate 100, tissue of prostate 100 applies a frictional force to implants 2022 and 2024. In some embodiments, in order to reduce the effect of the frictional force applied to implants 2022 and 2024, implants 2022 and 2024 are coated with a low-friction coating, e.g., PTFE (Teflon), MoST, ADLC or the like. In some embodiments, the implant surface is polished, e.g., electro-polished, mechanically polished, or otherwise, to reduce friction as implants 2022 and 2024 is advanced through the tissue of the patient.

Delivery tool 22 is reversibly couplable to implants 2022 and 2024. Tool 22 provides (1) a first implant holder 2021 which is reversibly coupled to a distal end 2030 of implant 2022, and (2) a second implant holder 2023 which is reversibly coupled to a distal end 2032 of implant 2024.

Implants 2022 and 2024 comprise a flexible material, e.g., nitinol. Typically, during advancement of implants 2022 and 2024 through urethra 60, implants 2022 and 2024 are compressed within a retractable sheath (not shown for clarity of illustration). Once advanced into bladder 80, the retractable sheath is retracted to expose implants 2022 and 2024 which expand radially upon retraction of the sheath. The retractable sheath is controllable by mechanical elements disposed on body 21 of tool 22, as described hereinabove.

FIG. 20A shows implants 2022 and 2024 in their spatial configuration after they have been advanced into bladder 80 and expanded therein. Upon expansion, implants 2022 and 2024 define a lumen having a diameter, e.g., between 2.5 mm and 15 mm, that is larger than a diameter of urethra 60. Tool 22 is retracted slightly so that the proximal ends of implants 2022 and 2024 are disposed at bladder neck 64 of bladder 80.

FIG. 20B shows partial implantation of implants 2022 and 2024 around urethra 60. Implant 2022 is shaped to provide a pointed proximal tip 2025 which punctures tissue of prostate 100. Implant 2024 is shaped to provide a pointed proximal tip 2027 which punctures tissue of prostate 100. In some embodiments, tips 2025 and 2027 comprise a rigid material, e.g., stainless steel, which is welded to the proximal ends of implants 2022 and 2024, respectively.

During implantation, (1) implant 2022 is rotated by holder 2021 in a counter-clockwise direction, as indicated by arrow 2, while, substantially at the same time, (2) implant 2024 is rotated by holder 2023 in a clockwise direction, as indicated by arrow 1. Implants 2022 and 2024 are implanted substantially at the same time around urethra 60. Implantation of implants 2022 and 2024 in counter-clockwise and clockwise directions, respectively, helps reduce a torsion force of implants 2022 and 2024 on tissue of prostate 100. That is, implant 2024 rotates tissue of prostate 100 clockwise (i.e., in a direction opposite the direction of implantation of implant 2022), and thereby balances the twisting of tissue 100 in a counter-clockwise direction in response to the implantation of implant 2022.

FIG. 20C shows continued implantation of implants 2022 and 2024 around urethra 60 in opposing rotational directions.

FIG. 20D shows implants 2022 and 2024 in their implanted states in which implant 2024 is disposed concentrically within implant 2022. Implants 2022 and 2024 support a post-operative diameter of urethra 60 in a in an unconstricted state.

It is to be noted that implants 2022 and 2024 are implanted in a distal-to-proximal direction from within bladder 80 by way of illustration and not limitation. For example, implants 2022 and 2024 may be implanted in a proximal-to-distal direction from within urethra 60 of the patient. In such an embodiment, implants 2022 and 2024 are coupled to tool 22 by their proximal ends, which the respective distal ends of implants 2022 and 2024 comprise pointed tips which puncture tissue of the urethra.

It is to be additionally noted that in some embodiments, implants 2022 and 2024 are implanted successively around urethra 60. In such an embodiment, implants 2022 and 2024 may be advanced through urethra 60 at different times. Alternatively, implants 2022 and 2024 may be disposed at respective longitudinal positions with respect to shaft 25 of delivery tool 22. In either embodiment, implants 2022 and 2024 are made to assume the spatial configuration (i.e., concentrically disposed) when implanted around urethra 60.

It is to be further noted that coiled implants 2022 and 2024 are shown by way of illustration and not limitation and that coiled implants 2022 and 2024 may be shaped to define any implant described herein. For example, coiled implants 2022 and 2024 may each be shaped to define implant 1200 as described hereinabove with reference to FIGS. 13A-D.

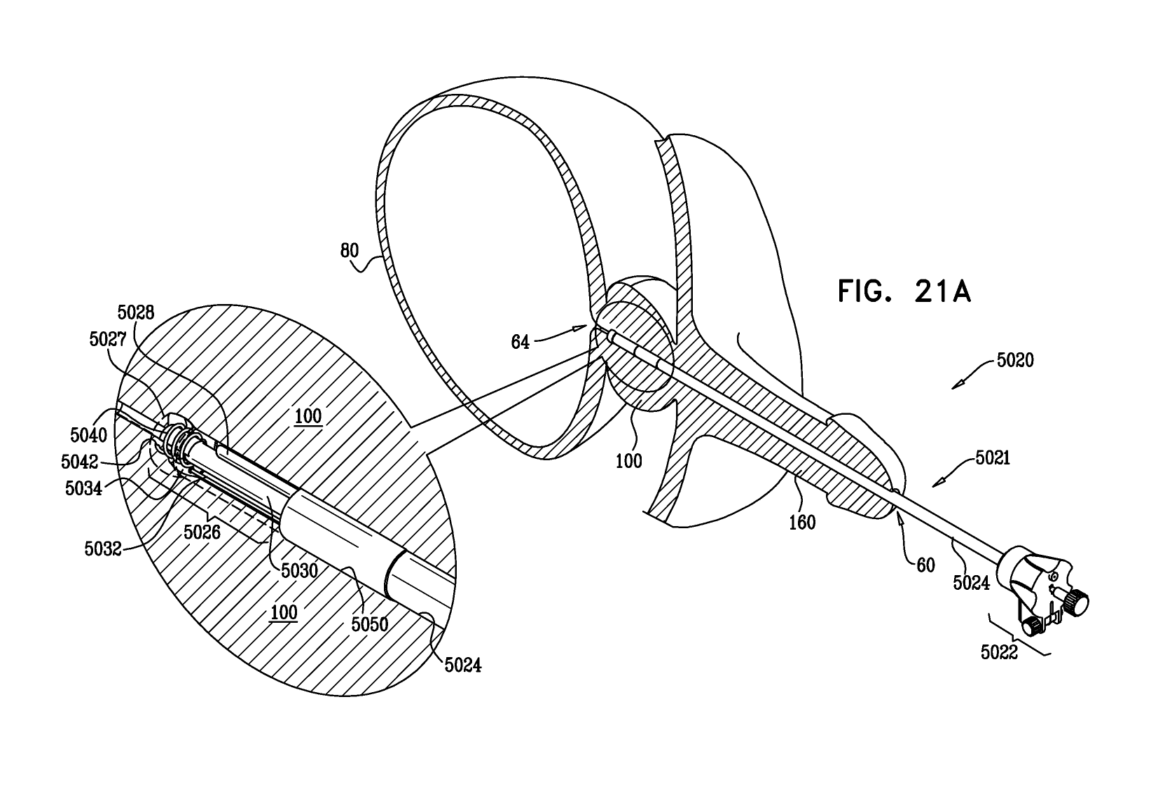

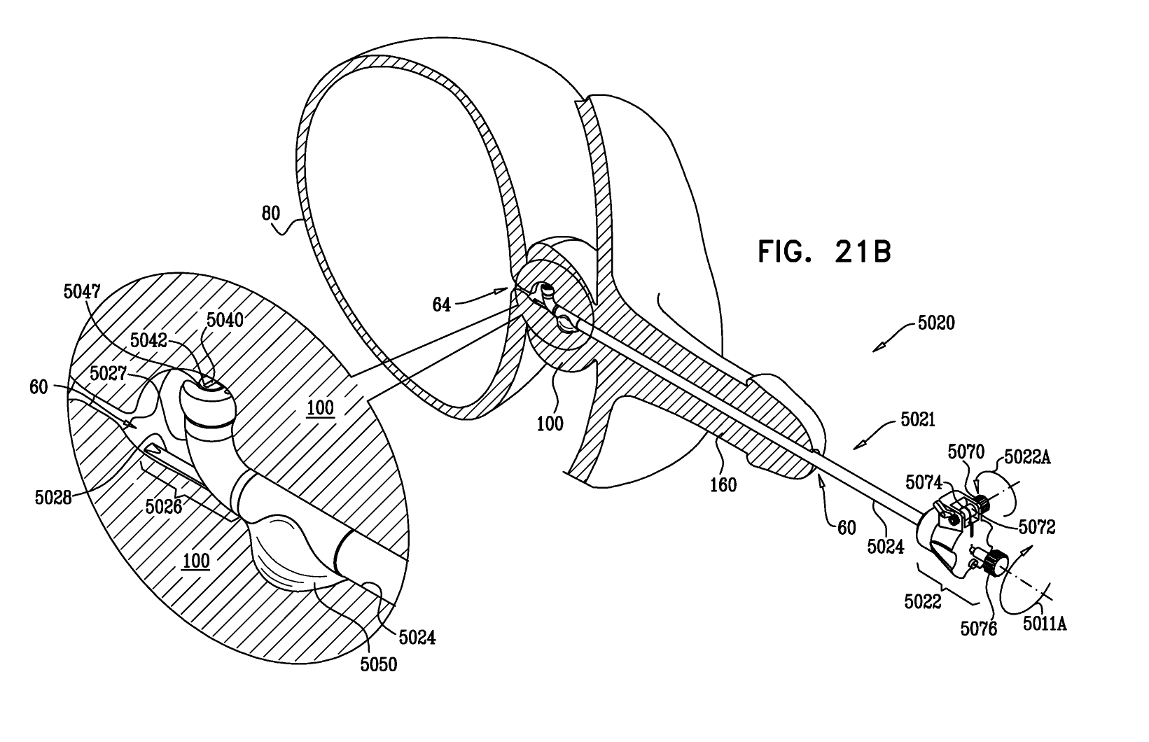

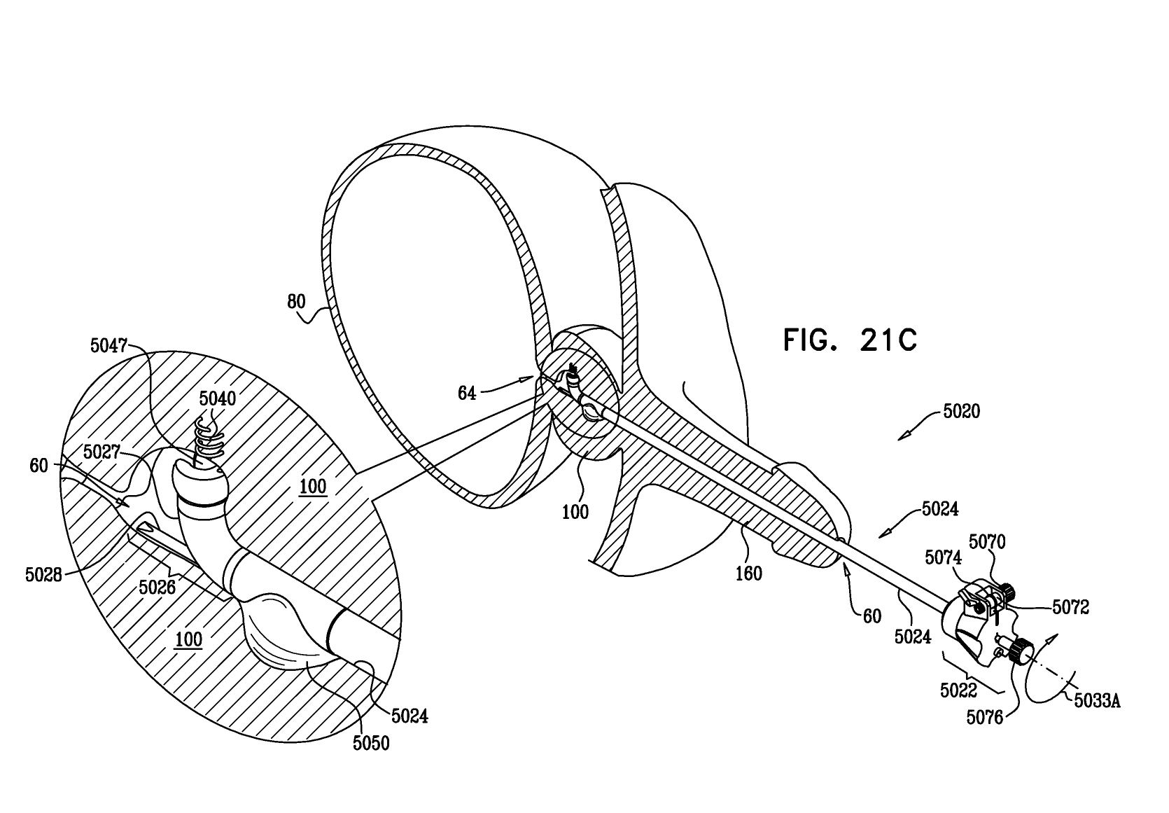

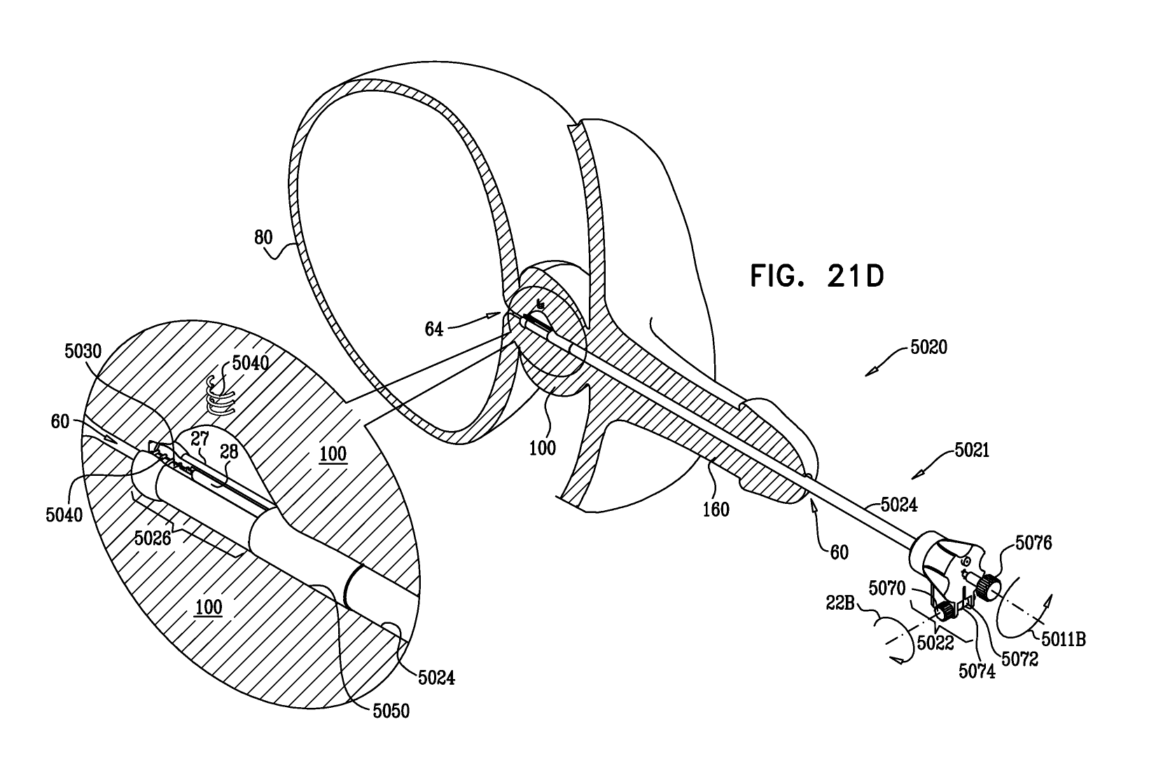

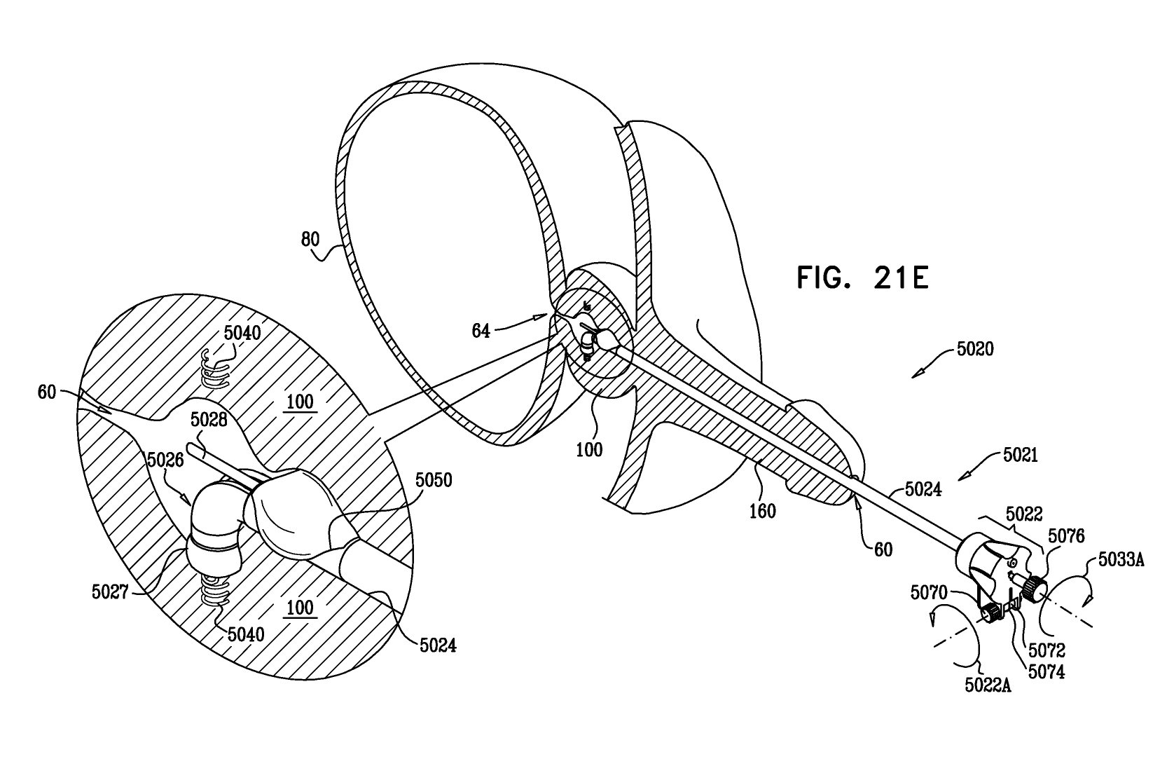

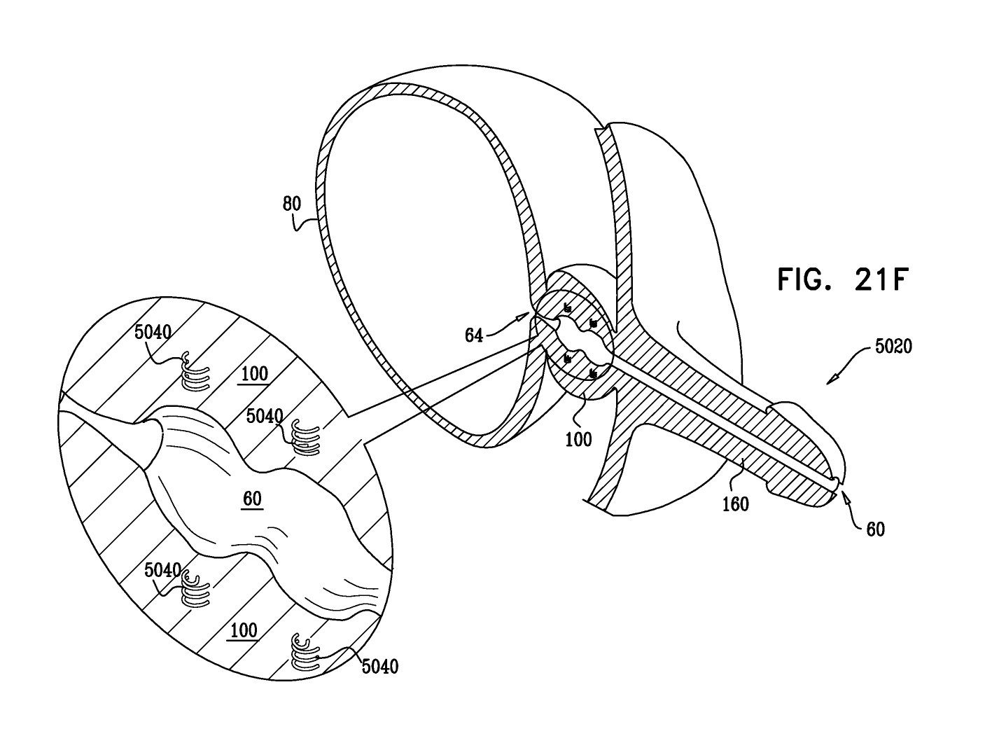

Reference is made to FIGS. 21A-F, which are schematic illustrations of a system 5020 comprising a transurethral delivery tool 5021 housing at least one coiled implant 5040, in accordance with an application of the present invention. Delivery tool 5021 comprises a handle 5022 and a delivery tool shaft 5024, and is configured to be inserted into a urethra 60 of a penis 160 of a patient. Shaft 5024 houses a deflectable shaft 5030 having a distal end that is deflectable from a longitudinal axis of delivery tool 5021. Delivery tool 5021 comprises a flexible, deflectable distal portion 5026 comprising a sleeve 5027 which surrounds distal portion 5026 of delectable shaft 5030. A distal ring 5034 is coupled to and surrounds a distal end of portion 5026 of shaft 5030. Deflection of distal portion 5026 is controllable by a pull-wire 5032 which is coupled (a) at a distal end thereof to ring 5034, and (b) at a proximal end thereof to handle 5022 of tool 5021. Pull-wire 5032 is manipulated by the operating physician via a tool-deflection-actuation system provided by handle 5022.

A portion of urethra 60 at prostate 100 is constricted due to pressure exerted thereupon by prostate 100. Typically, prior to introducing delivery tool 5021 into urethra 60, an outer sheath (not shown for clarity of illustration) is advanced distally through a proximal end of urethra 60 and toward bladder neck 64 of the patient. The outer sheath expands urethra 60 at prostate 100 as it is distally advanced toward bladder 80 of the patient. Typically, the outer sheath is advanced via an introducer tube (not shown) having a rounded proximal end which facilitates atraumatic advancement of the outer sheath through urethra 60. The outer sheath is advanced along urethra 60 prior to the advancement of delivery tool shaft 5024, thus creating an open passageway for the subsequent insertion of delivery tool 5021. Typically, the outer sheath is hollow and enables passage of tools through the urethra by providing a working channel. Delivery tool shaft 5024 is advanced distally through the outer sheath and toward bladder 80 of the patient. Typically, shaft 5024 comprises a hollow lumen for passing substances and/or tools therethrough, such as but not limited to, medications, fiber optics, biopsy tools, optical devices (e.g., CCD) and/or other imaging devices.

Delivery tool 5021 houses one or more implants 5040 within shaft 5024 of tool 5021. For some applications, a plurality of implants 5040 are disposed within shaft 5024. At a given time, a single implant 5040 is disposed within flexible sleeve 5027 of tool 5021 and surrounds a portion of distal portion 26 of deflectable shaft 5030.

Implant 5040 comprises a coiled implant comprising a proximal coil at a proximal end thereof, a distal coil at a distal end thereof, and a plurality of successive contiguous coils disposed between the proximal and distal coils. The distal coil of implant 5040 comprises a pointed tip 5042 which punctures tissue of prostate 100 during implantation of implant 5040. Ultimately, both the distal and proximal coils are disposed entirely within prostate tissue of the patient. That is, the distal coil does not extend beyond the prostate capsule (the capsule that surrounds the prostate), and the proximal coil does not extend into the urethra.

For some applications, implant 5040 comprises an expandable implant, e.g., a coil. Implant 5040 typically comprises a flexible biocompatible material, e.g., nitinol or silicone. Alternatively, implant 5040 is rigid. During transurethral advancement, implant 5040 is disposed in a compressed state thereof within tool 5021. Implant 5040 comprises a wire having a circular cross-section, by way of illustration and not limitation. For example, the wire of implant 5040 may be shaped to define any other suitable shape, e.g., a square, a triangle, a diamond, or an ellipse, in cross-section thereof. Typically, the shape of the wire forming the coiled implant helps facilitate pinching of tissue of the patient between the successive coils of implant 5040 during implantation thereof.

Delivery tool 5021 houses an imaging device 5028, e.g., a fiber optic scope or a cystoscope, which extends from handle 5022 toward sleeve 5027 of distal portion 5026 of tool 5021. Sleeve 5027 is shaped to define a slit in which a distal portion of imaging device 5028 is positioned such that, during deflection of distal portion 5026 of deflectable shaft 5030 and sleeve 5027, (a) sleeve 5027 is moved away from the distal portion of imaging device 5028 and (b) imaging device 5028 is freed from sleeve 5027 and remains disposed in parallel with respect to the longitudinal axis of urethra 60. Typically, imaging device 5028 comprises a side-viewing imaging device configured for imaging urethra 60 during implantation of implant 5040. Alternatively or additionally, an optical sensor, e.g., CCD, CIS, or CMOS, is coupled to a distal portion of shaft 5024 of delivery tool 5021.

FIG. 21B shows the deflection of sleeve 5027 and distal portion 5026 of tool 5021. Prior to the deflection, the entire tool 5021 is rotated by the physician 180 degrees, in the direction as indicated by arrow 5011A. The rotation prior to the deflection of distal portion 5026 and the implantation of implant 5040 is shown by way of illustration and not limitation. For example, the following steps for implanting implant 5040 may be performed without initially rotating tool 5021 by 180 degrees.

Handle 5022 comprises a deflection-actuation system comprising a knob 5070 coupled to a spool 5072. As described hereinabove with reference to FIG. 21A, pull-wire 5032 is coupled at a distal end thereof to distal portion 5026 of delectable shaft 5030 by being coupled to ring 5034 that surrounds a portion of distal portion 5026 of shaft 5030. A proximal portion 5074 of pull-wire 5032 is coupled to spool 5072 of the deflection-actuation system of handle 5022. Upon rotation of knob 5070 in the direction as indicated by arrow 5022A, portion 5074 of pull-wire 5032 is wrapped around spool 5072 thereby pulling on pull-wire 5032 and effecting tension in pull-wire 5032. Consequently, the distal portion of pull-wire 5032 pulls ring 5034 that is coupled to distal portion 5026 of deflectable shaft 5030, which causes distal portion 5026 of shaft 5030 to be pulled proximally, as shown.

As distal portion 5026 of shaft 5030 is pulled by pull-wire 5032, a flexible, distal tip of portion 5026 is deflected radially (e.g., by 90 degrees, as shown) from a position that is aligned with a longitudinal axis of tool 5021. During the deflection, flexible distal tip of portion 5026 slides along a wall of urethra 60 and compresses prostate tissue by pushing the wall of urethra 60.

Tool 5021 comprises an inflatable element 5050, e.g., a balloon, at a site proximal to flexible distal portion 5026. During the deflection of distal portion 5026 and subsequent implantation of implant 5040, inflatable element 5050 is inflated to push against and apply pressure to a wall of urethra 60 in order to stabilize and maintain in place tool 5021 during the deflection of portion 5026 and the subsequent implantation of implant 5040. Inflatable element 5050 has a volume in an inflated state thereof that is up to 50 cc, e.g., up to 5 cc. For some applications, inflatable element 5050 comprises an annular inflatable element that surrounds a distal portion of delivery tool 5021.

It is to be noted that an inflation conduit (not shown for clarity of illustration) is coupled at a distal end thereof to inflatable element 5050 and extends through the lumen of shaft 5024 and toward handle 5022 of tool 5021. When the physician desires to inflate element 5050, pressurized fluid is delivered via the conduit toward inflatable element 5050 from a fluid source that is disposed outside the body of the patient.

Typically, tool 5021 is preloaded with a plurality of implants 5040, which are disposed within shaft 5024 and surround deflectable shaft 5030. Typically, implants 5040 are successively disposed and surround the portion of shaft 5030 which is not configured for deflection, i.e., the portion of shaft 5030 that is proximal to deflectable portion 5026. Prior to deflection of distal portion 5026 of tool 5021, a first one of implants 5040 is pushed by an elongate pushing tool (not shown for clarity of illustration) disposed within shaft 5024, toward a position within the distal tip of portion 5026. Once positioned at the distal tip of portion 5026, the proximal coil of implant 5040 is engaged by an elongate, flexible screwdriver tool 5047 that is disposed within a lumen of shaft 5030. Implant 5040 is thereby primed for implantation in tissue of prostate 100. Screwdriver tool 5047 defines a distal portion that is flexible and deflectable together with distal portion 5026 of shaft 5030.

FIG. 21C shows implantation of a first implant 5040 in tissue of prostate 100 that has been compressed by the distal tip of tool 5021 in response to the deflection of distal portion 5026. As shown, following deflection of portion 5026, the distal tip of portion 5026 is positioned perpendicularly with respect to the wall of urethra 60 and in alignment with a vicinity of prostate 100 designated for implantation of implant 5040. That is, prior to implantation, the deflected portion 5026 moves implant 5040 along an axis which will ultimately define the longitudinal axis of implant 5040 upon implantation of implant 5040 in tissue of prostate 100.

Handle 5022 comprises an implant-actuation-system comprising a knob 5076 that is rotatable by the operating physician in order to corkscrew implant 5040 into tissue of prostate 100. Rotation of knob 5076 controls the rotation of screwdriver tool 5047 to effect corkscrewing of implant 5040 into tissue of prostate 100.

As described hereinabove with reference to FIG. 1A, the distal coil of implant 5040 is shaped to provide a pointed tip 5042 configured to puncture tissue of prostate 100. For some applications, pointed tip 5042 comprises a pointed needle which is coupled to (e.g., soldered to or attached using any other applicable attachment means) the distal coil of implant 5040. Typically, the needle of tip 5042 comprises a rigid, biocompatible material, e.g., stainless steel, configured to facilitate ongoing penetration of implant 5040 as it is advanced through tissue of prostate 100. It is to be noted that the needle is shaped to define any suitable shape configured for cutting/penetrating tissue.

For some applications, as pointed tip 5042 of implant 5040 is advanced through the tissue of the patient, it is configured to ablate the tissue. Implant 5040 may be coated with a substance, such as but not limited to, (a) a medication (e.g., an antibiotic) or (b) an electrical insulator (e.g., Teflon). A distal portion of implant 5040, i.e., one or more of the coils, may be energized to deliver RF energy, for example, to ablate tissue. For some applications, the distal portion of implant 5040 is coupled to an electrode. Additionally or alternatively, the distal portion of implant 5040 may be energized to provide ultrasound or thermal energy (e.g., heating or cooling.(

For some applications, implant 5040 comprises a hollow, helical implant shaped to define a helical lumen and at least one hole, e.g., a plurality of holes, at the proximal end thereof. A fluid, e.g., saline, is typically injected at high pressure through the lumen of the hollow, helical implant and externally to the implant via the at least one hole, in order to cut tissue near the proximal tip of the implant as it advances through the tissue.

For some applications, a laser fiber is passed through the lumen of the hollow, helical implant 5040 and through the hole at the proximal end thereof. Typically, the laser fiber ablates tissue in the path of the implant as it is advanced therethrough. For some applications, an insulated RF transmitting wire (i.e., having a non-insulated transmitting-tip) is advanced through the helical lumen of the hollow implant.

For some applications, a fluid is passed through the lumen of the hollow, helical implant 5040. The hollow, helical implant is shaped to define holes (e.g., typically toward the proximal end of the implant) for release of the fluid externally to the implant. For some applications, the fluid comprises a lubricant which passes externally to the implant via the holes defined thereby in order to reduce a frictional force between the tissue and the implant.

Deflection of tool 5021 places the distal tip of tool 5021 in a position in which screwdriver tool 5047 implants implant 5040 in tissue of prostate 100 at a non-zero angle, e.g., 90 degrees, as shown, with respect to the longitudinal axis of urethra 60. Knob 5076 is rotated by the physician, in the direction as indicated by arrow 5033A, in order to rotate screwdriver tool 5047 and thereby effect implantation of implant 5040. Once implant 5040 is fully implanted in prostate 100, it is embedded entirely within tissue of prostate 100, i.e., a portion thereof is not disposed external to the capsule of prostate 100. That is, both the distal and proximal coils of implant 5040 are disposed within tissue of prostate 100.

Typically, as implant 5040 is advanced through tissue of prostate 100, tissue of prostate 100 applies a frictional force to implant 5040. For some applications, in order to reduce the effect of the frictional force applied to implant 5040, implant 5040 is coated with a low-friction coating, e.g., PTFE (Teflon), MoST, ADLC or the like. For some applications, the implant surface is polished, e.g., electro-polished, mechanically polished, or otherwise, to reduce friction as implant 5040 is advanced through the tissue of the patient.

Following the corkscrewing of implant 5040 in tissue of prostate 100, implant 5040 is decoupled from screwdriver tool 5047 and from delivery tool 5021 and maintains the tissue in a compressed state in order to enlarge the perimeter of urethra 60 in the vicinity of implant 5040. Typically, the tensile force of coiled implant 5040 maintains the tissue in the compressed state.

FIG. 21D shows deflectable portion 5026 of tool 5021 returning to a position that is parallel with respect to the longitudinal axis of tool 5021. This is done by rotation of knob 5070 of the tool-deflection-actuation system in the direction as indicated by arrow 5022B (i.e., in the direction opposite the direction used to pull distal portion 26 proximally, as indicated by arrow 5022A, with reference to FIG. 21B). Rotating knob 5070 in the direction as indicated by arrow 5022B unwinds portion 5074 of pull-wire 5032 from spool 5072 thereby loosening pull-wire 5032 and releasing the pulling force on the distal tip of deflectable portion 5026. Once deflectable portion 5026 is returned to a position that is parallel with the longitudinal axis of tool 5021, imaging device 5028 is returned within the slit defined by sleeve 5027. As shown, even after the distal tip of deflectable portion 5026 has been moved away from the wall of urethra 60, implant 5040 maintains the tissue of prostate 100 in a compressed state. This creates an enlarged perimeter of urethra 60 in the vicinity of implant 5040, as shown.

Inflatable element 5050 is deflated so as to release the stabilizing pressure force it exerts on the wall of urethra 60 during implantation of implant 5040.

Tool 5021 is then rotated 180 degrees with respect to the longitudinal axis thereof, in a direction as indicated by arrow 5011B (i.e., in the direction opposite the direction used to initially rotate tool 180 degrees, as indicated by arrow 5011A, with reference to FIG. 21B.(

As shown in FIG. 21E, a second implant 5040 is implanted in tissue of prostate 100 in a vicinity of prostate 100 that is opposite the site of implantation of the first implant 5040. Prior to implantation, the second implant 5040 is advanced distally within shaft 5024 to a position at the distal tip of deflectable portion 5026 such that implant 5040 is primed for implantation. Once positioned at the distal tip of deflectable portion 5026, screwdriver tool 5047 is coupled to implant 5040 at a distal portion thereof. Inflatable element 5050 is then inflated to stabilize and maintain the position of tool 5021 during the subsequent implantation of second implant 5040.

Knob 5070 of the tool-deflection-actuation system is rotated in the direction as indicated by arrow 5022A in order to radially deflect deflectable portion 5026 in a manner as described hereinabove with reference to FIG. 21B. Responsively, the distal tip of deflectable portion 5026 is slid along the wall of urethra 60 while radially pushing the wall of urethra 60 and the prostate tissue. Once the wall of urethra 60 is pushed and the tissue of prostate 100 is compressed, knob 5076 of the implant-actuation-system is rotated in the direction as indicated by arrow 5033A in order to drive screwdriver tool 5047 to corkscrew implant into tissue of prostate 100, as described hereinabove with respect to the implantation of first implant 5040 with reference to FIG. 21C. Second implant 5040 is also positioned in tissue of prostate 100 at a non-zero angle (e.g., 90 degrees, as shown) with respect to the longitudinal axis of urethra 60.

The steps for deflection of tool 5021 and implantation of implants 5040 are repeated until all implants, e.g., four, as illustrated by way of illustration and not limitation in FIGS. 21A-F, have been implanted in tissue of prostate 100. It is to be noted that any suitable number of implants 5040, e.g., between 1 and 9 implants, may be implanted in tissue of prostate 100. Typically, delivery tool 5021 implants implants 5040 by orienting implants 5040 radially with respect to the urethra. As shown, delivery tool 5021 implants each of the plurality of implants 5040 at respective transverse planes of urethra 60 that are disposed along the longitudinal axis of urethra 60.

FIG. 21F shows four coiled implants 5040 implanted in respective implantation vicinities of the tissue of prostate 100. Once all four implants have been implanted, delivery tool 5021 is withdrawn from within urethra 60 of the patient. For every vicinity in which implant 5040 is implanted, the respective implant 5040 maintains the tissue in the vicinity in a compressed state thereof even after delivery tool 5021 has been withdrawn. As such, the perimeter of urethra 60 at each vicinity of prostate 100 is enlarged in response to the maintaining of the tissue in its compressed state by the respective coiled implant 5040, as shown.

The entirety of each implant 5040 is implanted in tissue of prostate 100. That is, no portion of any of implants 5040 is disposed within urethra 60 or outside the capsule of prostate 100.

It is to be noted that, although implants 5040 are implanted around urethra 60 symmetrically with respect to each other, as shown in FIG. 21F, implants 5040 may be positioned at any suitable location in prostate 100 and at any desired non-zero angle with respect to the longitudinal axis of urethra 60.

Following implantation of implants 5040 within prostate 100, a post-operative perimeter of the portion of urethra 60 at each implantation vicinity of prostate 100 is larger than the preoperative perimeter of the portion of urethra 60. Implants 5040 are generally rigid relative to the rigidity of the prostate. Implants 5040 thus support the urethral tissue, minimizing restenosis of urethra 60 should prostate 100 continue to enlarge.

Reference is again made to FIGS. 21A-F. Deflectable portion 5026 of tool 5021 facilitates independent control by the operating physician of the implantation of each implant 5040 in tissue of prostate 5040. The deflection of portion 5026 of tool 5021 enables specific targeting of a desired location of the prostate, by aligning the distal tip of deflectable portion 5026 to the portion of the wall of urethra 60 overlying the desired location of the prostate. Such alignment enables target-specific implantation of implants 5040. That is, deflectable portion 5030 facilitates the positioning of each implant at a desired location in prostate 100 and at a desired angle with respect to the longitudinal axis of urethra 60. As such, implants 5040 may be implanted in a manner which accommodates the dimensions and configurations of the lobes of the prostate of a given patient.

Reference is yet again made to FIGS. 21A-F. Implants 5040 may passively or actively contract following implantation in tissue of the prostate. For some applications, implants are made to adjust their configuration, e.g., contract, following implantation and in response to the application of energy thereto from an energy source (e.g., RF or ultrasound) which may be disposed (1) externally to the body of the patient, (2) in contact with the implant, or (3) internally to the patient's body but not in contact with the implant. For some applications, implants 5040 passively contract following implantation to further compress and pull the prostate tissue radially with respect to the longitudinal axis of the urethra. For example, each implant 5040 may be implanted in an expanded state thereof, in which the longitudinal length thereof is longer in its expanded state than in its resting state because the coils of each implant are distanced from each other. Once released from the delivery tool and implanted in tissue of the patient, implants 5040 contract to assume their resting state length, thereby pulling tissue in response to the contracting.

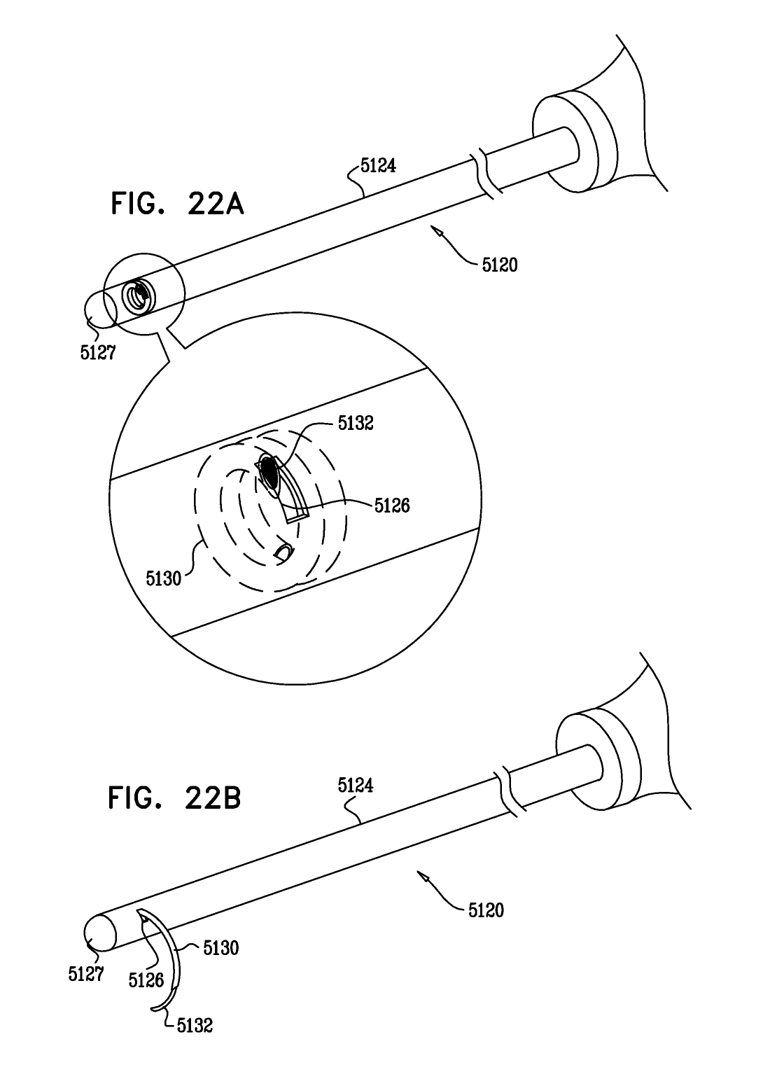

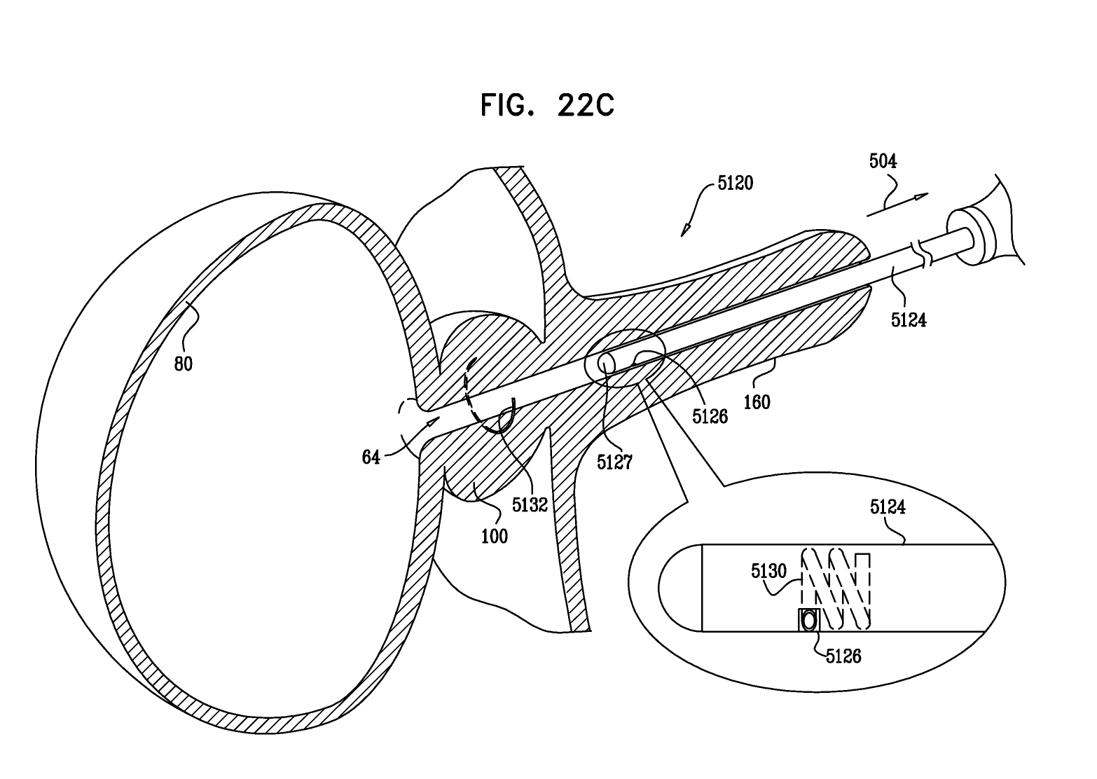

Reference is now made to FIGS. 22A-C, which are schematic illustrations of a system 5120 comprising a transurethral delivery tool 5124 and a curved implant 5132, in accordance with an application of the present invention. Delivery tool 5124 has a rounded distal end 5127 which facilitates atraumatic insertion of tool 5124 through urethra 60 and toward bladder 80. A portion of tool 5124 near distal end 5127 houses implant 5132 in a compressed state thereof, as shown in FIG. 22A. Implant 5132 is surrounded by an implantation-facilitating sleeve 5130, as described hereinbelow. Implant 5132 in a compressed state is shaped to define a coil in order to fit and be compressed within the lumen of delivery tool 5124. Similarly, sleeve 5130 is disposed in a compressed state and is shaped to define a coil in order to fit and be compressed within the lumen of delivery tool 5124. Once expanded from within the lumen of the delivery tool, implant 5132 is shaped to define an arc of up to 360 degrees, e.g., between 60 and 180 degrees. Following expansion, implant 5132 assumes its resting state, i.e., its uncompressed state.

Delivery tool 5124 is shaped to define an opening 5126 in a vicinity of distal end 5127 of tool 5124. As shown in FIG. 22B, implant 5132 surrounded by sleeve 5130, emerges from within the lumen of tool 5124 through opening 5126 of tool 5124. As implant 5132 and sleeve 5130 emerge from within the lumen of tool 5124, implant 5132 and sleeve 5130 expand from their compressed states to assume an arc of up to 360 degrees in its resting state.

Typically, sleeve 5130 surrounds implant 5132 during the initial implantation of implant 5132 in tissue of prostate 100. Sleeve 5130 comprises a material, e.g., stainless steel or nitinol, and by its physical construction is less flexible than implant 5132, which typically comprises a material such as nitinol. The rigidity of sleeve 5130 helps (1) push and compress tissue and puncture the tissue in order to create a channel in tissue of prostate 100 for receiving implant 5132, and (2) overcome the force of friction that prostate 100 applies to implant 5132 during implantation thereof.

FIG. 22B shows sleeve 5130 partially retracted with respect to a free end of implant 5132 (i.e., the end exposed from within sleeve 5130, as shown), leaving a portion of implant 5132 exposed from within sleeve 5130. Ultimately, sleeve 5130 is fully retracted within the lumen of the delivery tool and returns to its compressed state.

FIG. 22C shows implant 5132 in an implanted state within prostate 100 following implantation thereof from within urethra 60 by transurethral delivery tool 5124. As described hereinabove, as implant 5132 emerges from its compressed state within tool 5124, implant 5132 expands to assume its resting state. As implant 5132 expands, it pushes tissue of prostate 100 radially with respect to the longitudinal axis of urethra 60. Responsively to the pushing, the perimeter of urethra 60 at the vicinity of implant 5132 expands to a perimeter that is larger than the constricted, preoperative perimeter of urethra 60.

As shown in FIG. 22C, implant 5132 in its expanded, implanted state is shaped to define an implant plane having a normal thereto that is substantially parallel to the longitudinal axis of urethra 60. Although one implant 5132 is shown, it is to be noted that any number of implants may be implanted in a given transverse sectional plane of prostate 100, e.g., 1-9 implants. Additionally, a plurality of implants 5132 may be implanted in series along the longitudinal axis of urethra 60 at respective transverse planes thereof such that the plurality of implants 5132 resemble in spatial configuration at least a portion of a coiled implant.

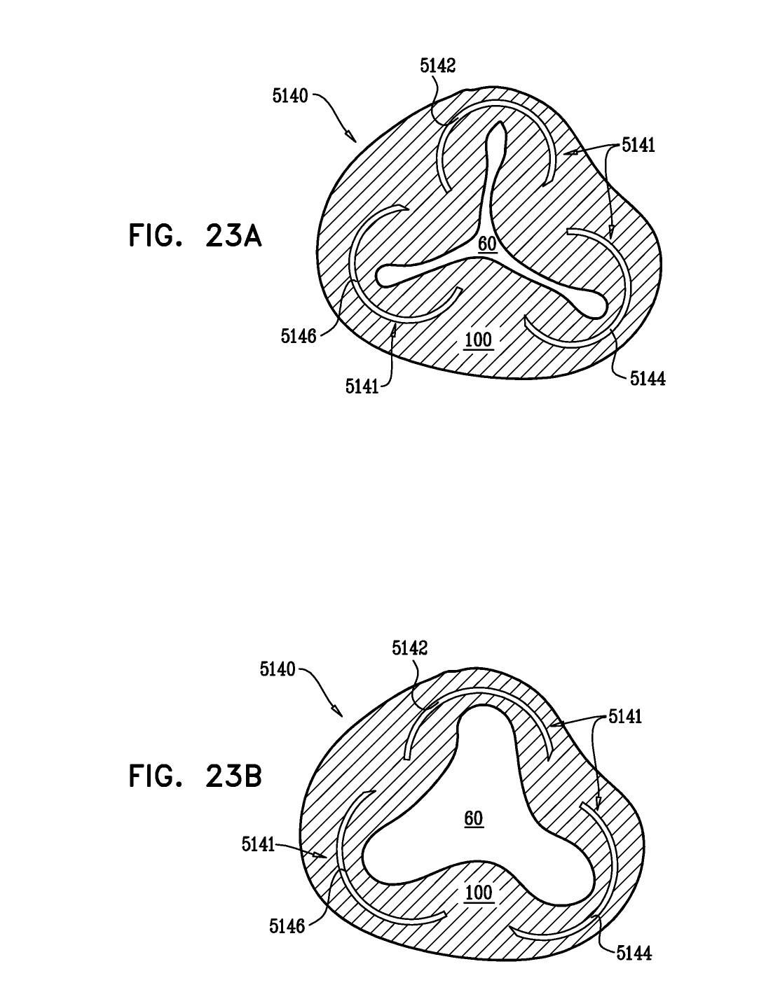

Implant 5132 maintains an enlarged perimeter at prostate 100, as shown. A plurality of implants 5132 may be implanted within prostate 100 in the same manner as described hereinabove. The delivery tool used to implant the plurality of implants 5132 implants the plurality of implants 5132 by orienting the implants radially with respect to urethra 60 along a single transverse plane of prostate 100, as shown by way of illustration in FIG. 23B, which is a schematic illustration of a system 5140 for maintaining an expanded perimeter of urethra 60 at prostate 100, in accordance with another application the present invention. Such a relative positioning of implants 5132 with respect to urethra 60 and prostate 100 helps ensure that the entirety of each implant 5132 is implanted within respective lobes of prostate 100 and that portions of each implant are not exposed within the lumen of urethra 60. Additionally, implanting implants 5132 that are shaped to define up to 360 degrees reduces the forces of friction of prostate 100 acting upon the implants as they are implanted in tissue of prostate 100. Each implant 5132 may be shaped differently so as to define different sized implants. For example, one implant may be shaped so as to define 270 degrees while the other may be shaped so as to define 90 degrees. The relative sizes of each implant 5132 accommodate the dimensions of a given lobe of the prostate in which at least a portion of each respective implant is implanted. That is, a first lobe may be larger at a given transverse sectional plane of prostate 100 than a second lobe. Thus, a larger implant may be implanted in the vicinity of the first lobe, while a smaller implant may be implanted in the vicinity of the second lobe. For some applications, a respective implant may be implanted entirely within a given lobe. The entirety of each implant 5132 is implanted in tissue of prostate 100. That is, no portion of any of implants 5132 is disposed within urethra 60 or outside the capsule of prostate 100.

Reference is again made to FIGS. 22A-B. Delivery tool 5124 and opening 5126 therein facilitates target-specific delivery of implant 5132 in tissue of prostate 100. That is, prior to implantation of implant 5132, tool 5124 is advanced until opening 5126 is positioned in alignment with a specific lobe of prostate or with a transverse sectional plane of prostate in which implant 132 is ultimately implanted.

Reference is again made to FIGS. 22A-C. It is to be noted that for some applications, tool 5124 comprises inflatable element 5050, as described hereinabove with reference to FIGS. 21A-F.

Reference is now made to FIGS. 23A-B which are schematic illustrations of a plurality of discrete, resilient curved implants 5141, in accordance with an application of the present invention. Implants 5141 comprise first, second, and third implants 5142, 5144, and 5146, respectively. Each implant 5141 is shaped to define up to 360 degrees. Each implant 5141 is implanted in prostate 100 such that it defines an implant plane having a normal thereto which is substantially parallel with respect to the longitudinal axis of urethra 60.

FIG. 23A shows implants 5141 implanted in a first configuration thereof in which each implant 5141 is shaped to define around 240 degrees, as shown, and has a first radius of curvature thereof. The delivery tool implants 5141 by placing implants 5141 adjacent to (e.g., around) a constricted urethra 60 and in a transverse sectional plane of prostate 100.

FIG. 23B shows implants 5141 in their expanded, resting states following implantation. Each implant 5141 enlarges to assume a second configuration in which implant 5141 defines around 180 degrees and a second radius of curvature that is larger than the first radius of curvature (as shown in FIG. 23A). While transitioning between the first and second configurations thereof, implants 5141 expand, and implants 5142 remodel and compress tissue of prostate 100 along a radius with respect to the longitudinal axis of urethra 60. The expansion of implants 5141 enlarges the perimeter of urethra 60 at the site of implantation of implant, i.e., the vicinity of the transverse plane in which implants 5141 are implanted. The perimeter of urethra 60 shown in FIG. 23B is larger than the perimeter of urethra 60 shown in FIG. 23A.

It is to be noted that for some applications, the radius of curvature of each implant 5141 may be larger in a resting state thereof (shown in FIG. 23B) than in a non-resting state thereof (shown in FIG. 23B).

Implants 5141 maintain an enlarged perimeter at prostate 100, as shown. Implants 5141 may be implanted within prostate 100 in the same manner as described hereinabove with reference to FIGS. 22A-B. Implants 5141 are implanted such that portions of neighboring implants are implanted in a given lobe of prostate 100. For example, a first portion of implant 5142 and a first portion of implant 5144 are implanted in a first lobe, and a second portion of implant 5144 and a first portion of implant 5146 are implanted in a second lobe of prostate 100. The relative positioning of implants 5141 with respect to urethra 60 and prostate 100, helps ensure that the entirety of each implant 5141 is implanted within respective lobes of prostate 100 and that portions of each implant 5141 are not exposed within the lumen of urethra 60. Additionally, implanting implants 5141 that are shaped to define up to 360 degrees reduces the forces of friction of prostate 100 acting upon the implants as they are implanted in tissue of prostate 100.

Each implant 5141 may be shaped differently so as to define different sized implants in their resting states thereof. For example, one implant may be shaped so as to define 270 degrees in a resting state thereof in awhile the other may be shaped so as to define 90 degrees in a resting state thereof. The relative sizes of each implant 5141 accommodate the dimensions of a given lobe of the prostate in which at least a portion of each respective implant is implanted. That is, a first lobe may be larger at a given transverse sectional plane of prostate 100 than a second lobe. Thus, a portion of an implant or a larger implant may be implanted in the vicinity of the first lobe, while a smaller portion of an implant or a smaller implant may be implanted in the vicinity of the second lobe.

For some applications, a respective implant may be implanted entirely within a given lobe and is sized in accordance with the dimensions of the lobe in which it is implanted. The entirety of each implant 5141 is implanted in tissue of prostate 100. That is, no portion of any of implants 5141 is disposed within urethra 60 or outside the capsule of prostate 100. In such an embodiment, each implant functions to independently remodel by compressing and maintaining in a compressed state tissue of the lobe in which the implant is implanted.

Although three implants 5141 are shown, it is to be noted that any number of implants may be implanted in a given transverse sectional plane of prostate 100, e.g., 1-9 implants. Additionally, a plurality of implants 5141 may be implanted in series along the longitudinal axis of urethra 60 at respective longitudinal planes thereof such that the plurality of implants 5141 resemble in spatial configuration at least a portion of a coiled implant.

Reference is again made to FIGS. 23A-B. It is to be noted that implants 5141 are shaped to define around 240 degrees in their first configuration and 180 degrees in their second configuration, by way of illustration and not limitation. For example, in the first configuration, implants 5141 may be shaped to define between 120 and 300 degrees, and, in their second configuration, between 60 and 240 degrees.

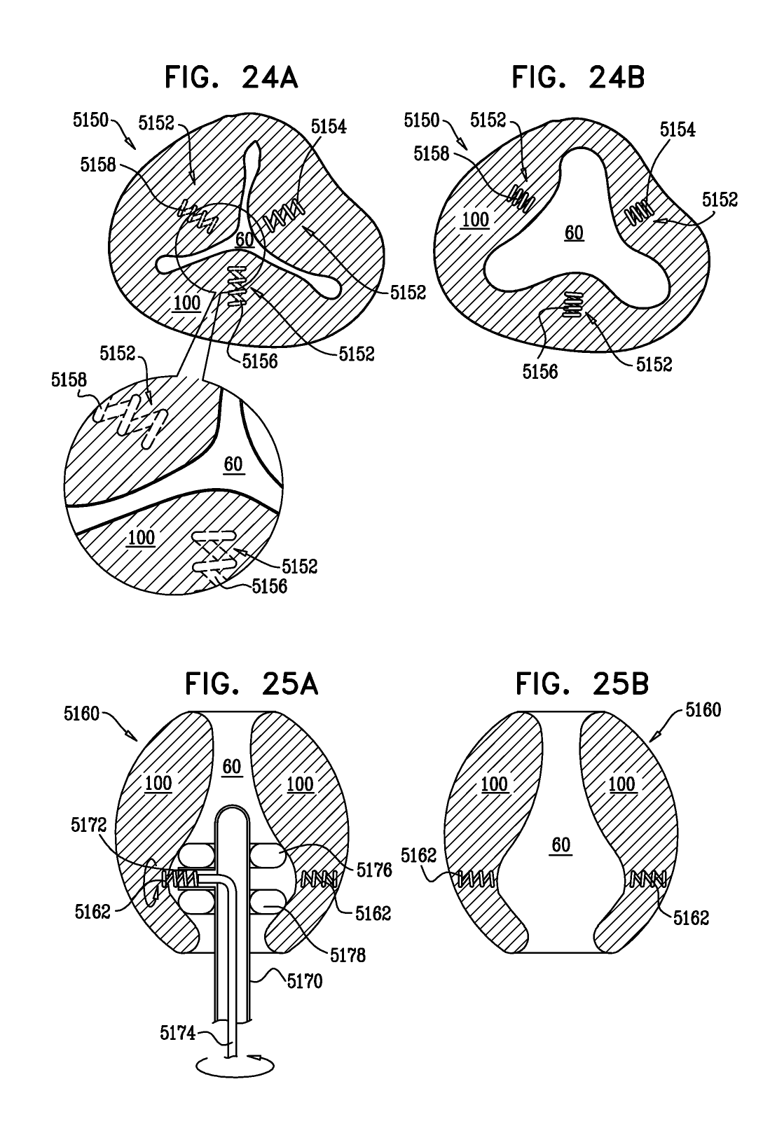

FIGS. 24A-B, show a system 5150 comprising coiled implants 5152 implanted at a non-zero (e.g., 90 degree) angle with respect to the longitudinal axis of urethra 60, in accordance with an application of the present invention. FIG. 24A shows a stage in an implantation procedure where a first implant 5154 has been implanted in a first lobe of prostate 100 in an expanded state thereof, and prior to second and third implants 5156 and 5158 (shown in phantom) being implanted in the prostate. FIG. 24B shows implants 5154, 5156, and 5158 in their contracted, resting states following their initial implantation.

For some applications, implants 5152 are made to contract following implantation and in response to the application of energy thereto from an energy source (e.g., RF or ultrasound), which may be disposed (1) externally to the body of the patient, (2) in contact with the implant, or (3) internally to the patient's body but not in contact with the implant. For such an application, following initial implantation, implants 5152 maintain the larger pitch between the successive coils (as shown in FIG. 24A) than the pitch of implants 5152 in their compressed state (as shown in FIG. 24B). This application of energy causes implants 5152 to compress and pull tissue of prostate 100 radially from the longitudinal axis of urethra 60.

Alternatively or additionally, other techniques are used to cause implants 5152 to compress following implantation. For example, each implant may be coated with or otherwise coupled to a biodegradable support structure that maintains the implant in the expanded state. Upon degradation of the biodegradable support structure, implants 5152 compress and pull tissue of prostate 100 radially from the longitudinal axis of urethra 60.

For some applications, implants 5152 comprise a shape memory alloy, e.g., nitinol. For such an application, implants 5152 may be cooled prior to implantation and are thereby deformed into an expanded (substantially not curved) configuration. Upon implantation, the implants reach body temperature, causing them to regain their original compressed shape, by contracting in response to the heat. This recovery of the original compressed shape causes implants 5152 to compress and pull tissue of prostate 100 radially from the longitudinal axis of urethra 60.

For some applications, the following procedure is used to implant implants 5152 as shown in FIGS. 24A-B or to implant other implants described herein. Implants 5152 are transurethrally implanted in tissue of prostate 100 using a delivery tool which houses implants 5152 and has a deflectable tip having an open distal end. The deflectable tip is steered such that the distal end is made to contact the wall of urethra 60 at a non-zero angle, e.g., between 40 and 160 degrees, with respect to the longitudinal axis of urethra 60. The delivery tool provides a lumen thereof which houses a corkscrewing tool having a flexible tip. In such an alignment implant 5152 may be corkscrewed by the corkscrewing tool into tissue of prostate 100 and implanted in alignment with the non-zero angle of the deflected tip of the delivery tool. The transitioning of implants 5152 from their expanded state (FIG. 24A) to their compressed state (FIG. 24B) compresses and pulls tissue of prostate 100 away from the longitudinal axis of urethra 60 in order to expand the perimeter of the wall of urethra 60, as shown in FIG. 24B.

For some applications (not as shown in FIGS. 24A-B), prior to implantation, the delivery tool is radially deflected to compress and push the wall of urethra 60 and the prostate tissue away from the longitudinal axis of urethra 60 (i.e., as described hereinabove with reference to FIGS. 21A-F). Implants 5152 maintain the expanded perimeter of urethra 60 caused by the pushing of the tissue.

Reference is again made to FIGS. 24A-B. It is to be noted that each implant 5152 is typically implanted in a respective lobe of prostate 100. (For some applications, a patient only has implant 5152 placed in one lobe, and does not have the other lobes treated with an implant.) In such a manner, the lobes of the prostate may be controlled individually and independently. In an embodiment, more than one implant 5152 may be implanted in tissue of a given lobe if appropriate.

Implants 5152 each comprise a coiled implant comprising a proximal coil at a proximal end thereof, a distal coil at a distal end thereof, and a plurality of successive contiguous coils disposed between the proximal and distal coils. The distal coil of each implant 5152 comprises a pointed tip which punctures tissue of prostate 100 during implantation of implant 5152. Ultimately, both the distal and proximal coils are disposed entirely within prostate tissue of the patient, i.e., the distal coil does not extend beyond the prostate capsule and the proximal coil does not extend into the urethra. The entirety of each implant 5152 is thus implanted in tissue of prostate 100.

Reference is made to FIGS. 21A-F and 24A-B. It is to be noted that implants 5040 of system 5020 may be implanted in prostate 100 in a similar relative spatial configuration of implants 5152 as shown in FIG. 24B. That is, each implant 5040 may be implanted in prostate 100 in a respective lobe of prostate 100.

Reference is now made to FIGS. 21A-F, 22A-C, 23A-B, and 24A-B. It is to be noted that delivery tool 5021 shown in FIGS. 21A-F may be used to implant the implants described herein. That is, deflectable portion 5026 of tool 5021 is used to first push the wall of urethra 60 and prostate tissue radially with respect to the longitudinal axis of urethra 60 prior to implantation of the implants described herein. Following implantation, the implants function to further enlarge and/or maintain the expanded perimeter of urethra 60 at the site of implantation.

FIGS. 25A-B show a system 5160 for transurethrally implanting coiled implants 5162 in tissue of prostate 100 using a delivery tool 5170 comprising annular inflatable elements 5176 and 5178 at a distal portion thereof, in accordance with an application of the present invention. Delivery tool 5170 is through urethra 60 until a distal portion thereof is positioned in urethra 60 in the vicinity of prostate 100. Once delivery tool 5170 is properly positioned, annular inflatable elements 5176 and 5178 are inflated in order to push against the wall of urethra 60. In response to the pushing, the prostate tissue surrounding the portion of the wall being pushed is compressed and pushed radially with respect to the longitudinal axis of urethra 60, thereby enlarging the perimeter of urethra 60 at the implantation site.

It is to be noted that respective inflation conduits (not shown for clarity of illustration) are coupled at a respective distal ends thereof to inflatable elements 5176 and 5178, respectively. The conduits extend through the lumen of shaft 5024 and toward handle 5022 of tool 5021. When the physician desires to inflate element 5050, pressurized fluid is delivered toward inflatable element 5050 via the conduits from a fluid source that is disposed outside the body of the patient.

The distal portion of tool 5170 is shaped to provide a lateral opening 5172 (e.g., a hole, or a channel, as shown). Tool 5170 is shaped so as to provide a lumen which houses implants 5162 (prior to their implantation) and a screwdriver tool 5174 which has a distal deflectable portion. Once appropriately positioned in urethra 60, and following inflation of elements 5176 and 5178, screwdriver exits opening 5172 and corkscrews an implant 5162 in tissue of prostate 100. Lateral opening 5172 facilitates implantation of implant 5162 at a non-zero angle (e.g., 90 degrees, as shown) with respect to the longitudinal axis of urethra 60. The entirety of implant 5162 is implanted in tissue of prostate 100. That is, no portion of implant 5162 is disposed within urethra 60 or outside the capsule of prostate 100.

As shown in FIG. 25B, following removal of delivery tool 5170 from within urethra 60, implants 5162 function to maintain (a) the prostate tissue in a compressed state, and (b) the perimeter of urethra 60 that has been enlarged by annular inflatable elements 5176 and 5178.

Although two implants 5162 are shown, it is to be noted that any number of implants may be implanted in a given transverse sectional plane of prostate 100, e.g., 1-9 implants. Additionally, a plurality of implants 5162 may be implanted in series along the longitudinal axis of urethra 60. It is to be noted that implants 5162 may comprise compressible implants, as described hereinabove with reference to FIGS. 24A-B. Compressible implants function to further compress tissue of prostate 100 and thereby further expand the perimeter of urethra 60 that has been expanded by annular inflatable elements 5176 and 5178.

Implants 5162 each comprise a coiled implant comprising a proximal coil at a proximal end thereof, a distal coil at a distal end thereof, and a plurality of successive contiguous coils disposed between the proximal and distal coils. The distal coil of each implant 5162 comprises a pointed tip which punctures tissue of prostate 100 during implantation of implant 5162. Ultimately, both the distal and proximal coils are disposed entirely within prostate tissue of the patient, i.e., the distal coil does not extend beyond the prostate capsule and the proximal coil does not extend into the urethra.

It is to be noted that delivery tool 5170 may be used to implant any of the implants described herein with reference to FIGS. 21A-F, 22A-C, 23A-B, 24A-B, and 26.

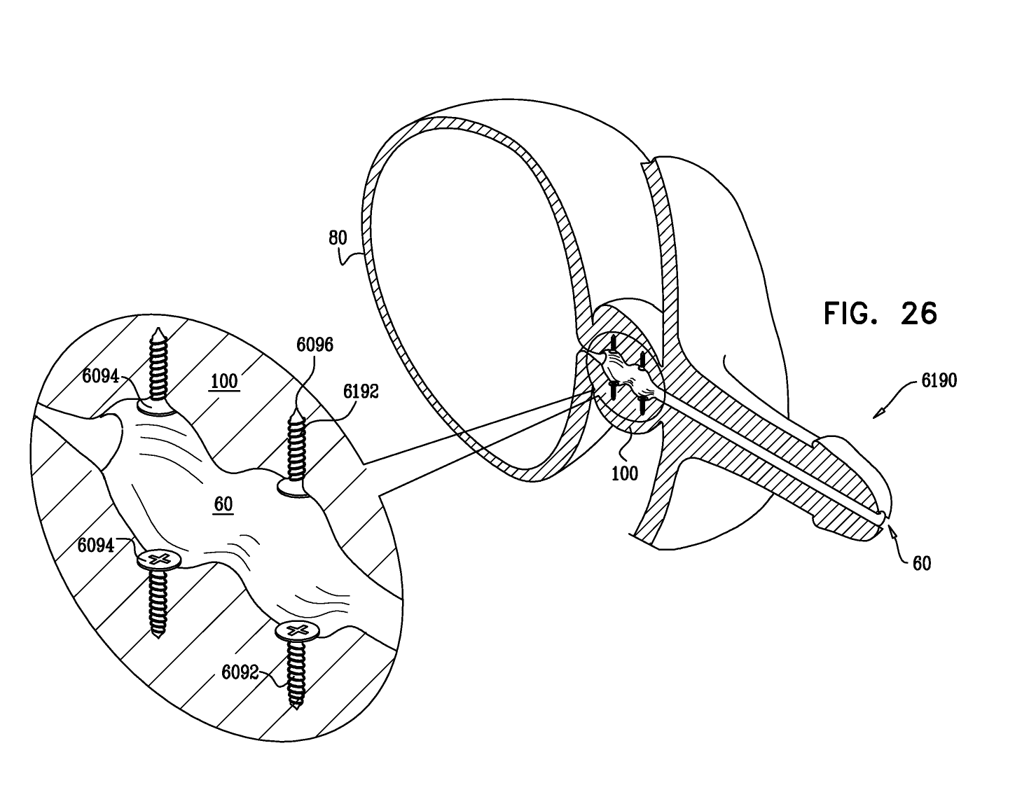

Reference is made to FIG. 26, which shows a system 190 comprising screw implants 6192 implanted generally perpendicularly with respect to the longitudinal axis of urethra 60, in accordance with an embodiment of the present invention. Implants 6192 are transurethrally implanted in tissue of prostate 100 using a delivery tool (not shown), which houses a plurality of implants 6192, e.g., four, and has a deflectable tip having an open distal end. The deflectable tip is steered such that the distal end is made to contact the wall of urethra 60 at a non-zero angle, e.g., 90 degrees, with respect to the longitudinal axis of urethra 60. The delivery tool is then radially deflected to compress and push the wall of urethra 60 and the prostate tissue away from the longitudinal axis of urethra 60 thus expanding the perimeter of the wall of urethra 60 (i.e., as described hereinabove with reference to FIGS. 21A-F). While the prostate is compressed, screw implant 6192 is screwed by a screwing tool into tissue of prostate 100 and implanted in alignment with the non-zero angle, e.g., 90 degrees, of the deflected tip of the delivery tool. Once screw implants 6192 have been implanted into compressed prostate tissue, the delivery tool is withdrawn from within urethra 60 of the patient. Implants 6192 maintain tissue of prostate 100 in a compressed state such that the expanded perimeter of urethra 60 in the vicinity of implants 6192 is maintained in an enlarged state following removal of the delivery tool.

It is to be noted that, although screw implants 6192 are implanted around urethra 60 symmetrically with respect to each other, as shown in FIG. 26, implants 6192 may be positioned at any suitable locations in prostate 100 and at any desired non-zero angle with respect to the longitudinal axis of urethra 60.

For some applications, implant 6192 comprises a screw implant comprising a screw head 6094 at a proximal end thereof, a pointed tip 6096 at a distal end thereof, and a screw body wrapped by a helical thread extending between the proximal and distal ends. The distal end of implant 6192 comprises a pointed tip 6096 which punctures tissue of prostate 100 during implantation of implant 6192. During implantation of screw implant 6192, the distal end is typically advanced into the prostate tissue until ultimately, the entire screw, excluding head portion 6094, is disposed within prostate tissue of a patient. That is, the head 6094 of screw implant 6192 remains within the urethra, secured to the wall of urethra 60. The distal end of implant 6192 does not extend beyond the prostate capsule (the capsule that surrounds the prostate). Alternatively, during implantation of screw implant 6192, the distal end is advanced into the prostate tissue until ultimately the entire screw is disposed within prostate tissue of a patient. That is, the distal end of implant 6192 does not extend beyond the prostate capsule (the capsule that surrounds the prostate), and the proximal end of implant 192 does not extend into the urethra.

Some or all of implant 6192 is generally rigid relative to the rigidity of the prostate, and typically comprises a biodegradable material, e.g., a biodegradable polymer, such as PLA and/or PGA. Thus, following implantation in the prostate, the biodegradable portion of screw implant 6192 gradually degrades into natural metabolites that are absorbed entirely in the body or secreted from the body, thereby reducing the risk of infection.

Screw implant 6192 may comprise in its body, or be coated with, a substance, such as but not limited to, a medication (e.g., an antibiotic and/or an anti-inflammatory medication). For some applications, the medication is intended for the treatment of benign prostatic hypertrophy. Examples for such medications are alpha adrenergic antagonists, e.g., Alfuzosin, Doxazosin mesylate, Tamsulosin and Terazosin. Thus, following implantation, these biodegradable medication-coated implants function (a) to maintain the expanded perimeter of urethra 60 at the site of implantation, and (b) to treat the prostatic tissue by releasing medication at the site of implantation as the implant disintegrates.

For some applications, screw head 6094 comprises a biodegradable material, e.g., PLA and/or PGA, and is typically coated with a medication, whereas, the screw body comprises a biocompatible material, configured for chronic implantation. Typically, during implantation, the screw body is advanced into the prostate tissue until ultimately it is entirely disposed within the prostate tissue, while the screw head 6094 remains in the urethra and is fixed to the wall of urethra 60. Following implantation of implant 6192 the screw body functions to maintain an expanded perimeter of urethra 60 at the site of implantation, by maintaining prostate tissue in a compressed state. The screw head gradually degrades, releasing medication, e.g., for treatment of benign prostatic hypertrophy. Alternatively, during implantation, the entire screw implant is disposed within prostate tissue of a patient. That is, the distal end of the implant does not extend beyond the prostate capsule, and the proximal end of the implant does not extend into the urethra. Typically, following implantation of the implant, the screw body functions to maintain an expanded perimeter of urethra 60 at the site of implantation, by maintaining prostate tissue in a compressed state. The screw head gradually degrades, releasing medication, e.g., for treatment of benign prostatic hypertrophy, directly into the prostatic tissue.

It is to be noted that any of the implants described herein with reference to FIGS. 21A-F, 22A-C, 23A-B, 24A-B, and 25A-B may be coated with a medication, such as but not limited to, a medication for treatment of benign prostatic hypertrophy.

Reference is again made to FIGS. 21A-F, 22A-C, 23A-B, 24A-B, and 25A-B. It is to be noted that delivery tools 5021, 5124, or 5170 may be used to implant any of the implants described herein with reference to FIGS. 21A-F, 22A-C, 23A-B, 24A-B, 25A-B, and 26.

Reference is again made to FIGS. 21A-F, 22A-C, 23A-B, 24A-B, 25A-B, and 26. The prostatic implants described herein may be coated with any low-friction coating as described herein. For example, the implants may be coated with a substance, such as but not limited to, a medication (e.g., an antibiotic) or with an electrical insulator (e.g., Teflon). The implants may be coated with low friction coatings, e.g., PTFE (Teflon), MoST, ADLC or the like. For some applications, the surfaces of the implants are polished, e.g., electro-polished, mechanically polished, or other, to reduce friction as the implants are implanted in the tissue of the patient. For some applications, a portion of each of the implants, i.e., one or more of the coils, may be energized to deliver RF energy, for example, to ablate tissue. For some applications, a portion of each one of the implants is coupled to an electrode. Additionally or alternatively, the portion of each implant may be energized to provide ultrasound or thermal energy (e.g., heating or cooling).

For some applications, implants described herein are coated with a pro-fibrotic agent, which helps enhance the anchoring of the implants in prostate 100.

Reference is again made to FIGS. 21A-F, 22A-C, 23A-B, 24A-B, 25A-B, and 26. The number of prostatic implants described herein is selected according to the needs of a given patient. A length of prostate 100 is measured prior to the implantation procedure such that a suitable number of implants, each having a desired length, is selected.

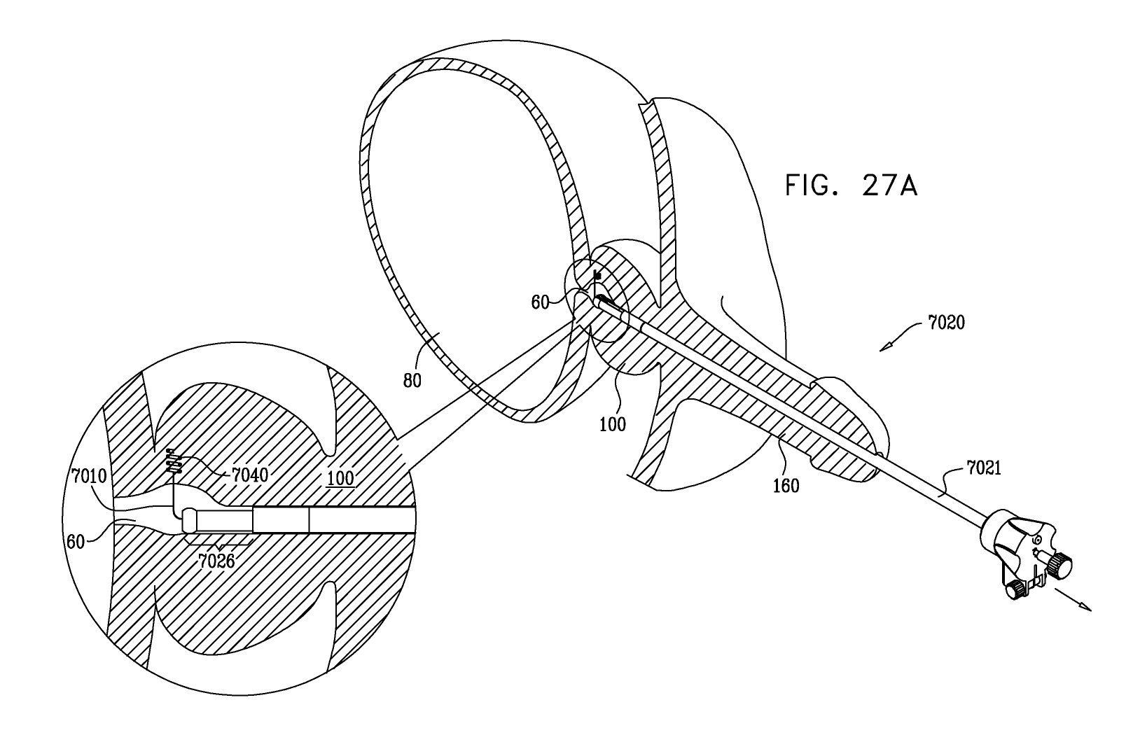

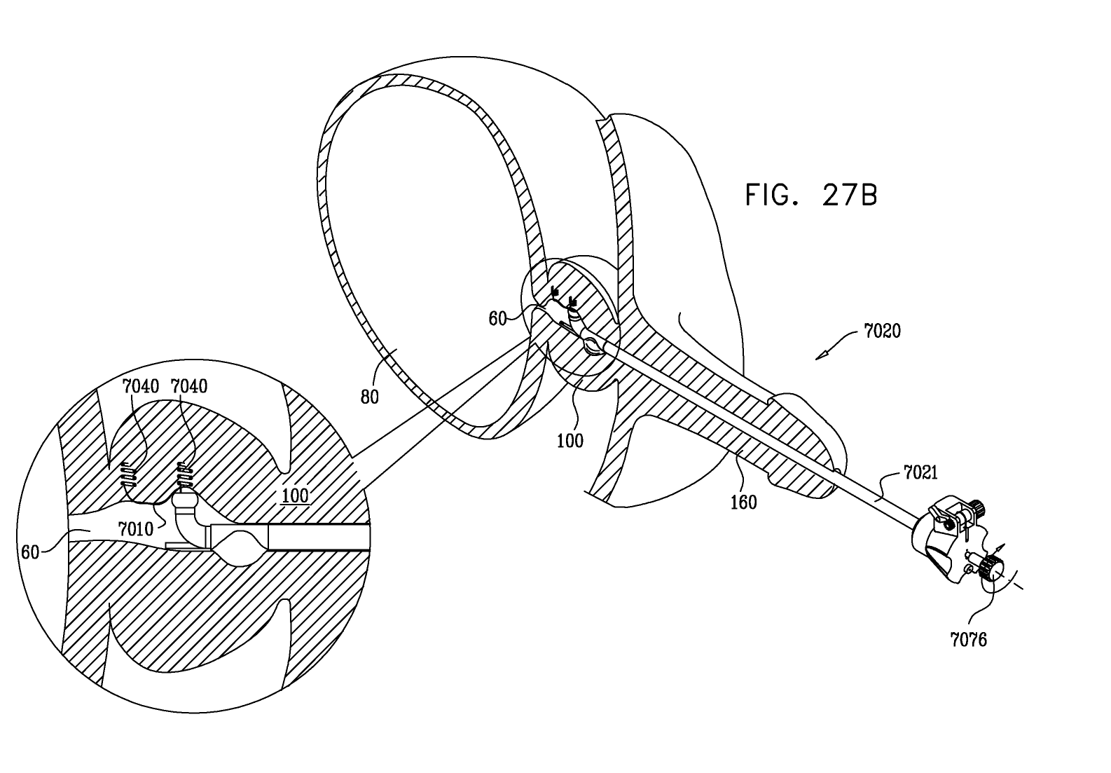

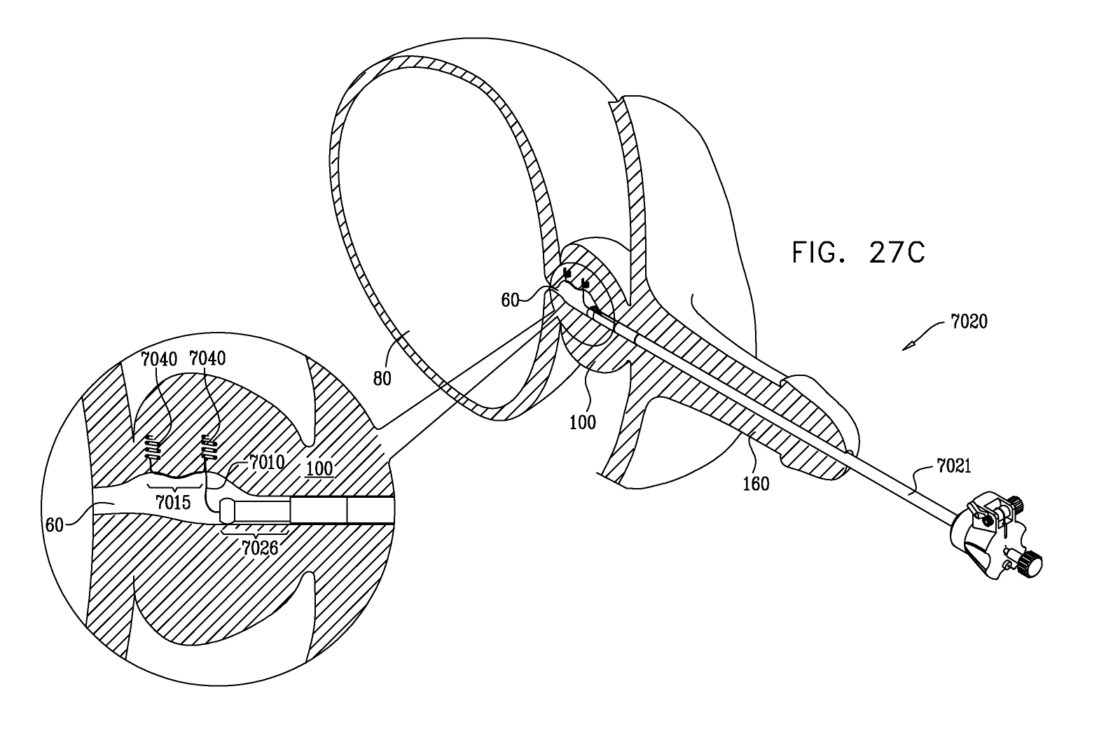

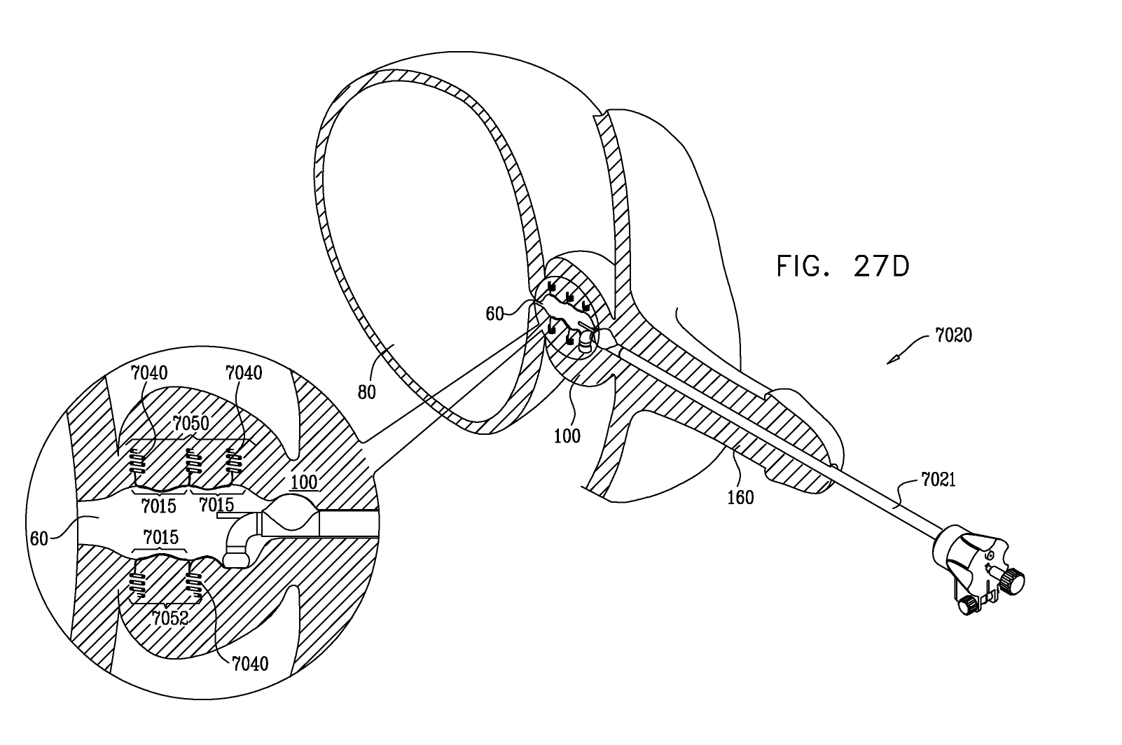

Reference is now made to FIGS. 27A-D which are schematic illustrations of a system 7020 comprising a transurethral delivery tool 7021 housing at least two implants 7040, typically coiled implants 7040, coupled to a wire 7010, in accordance with an application of the present invention. It is to be noted that wire 7010 is shown by way of illustration and not limitation, and that any suitable flexible longitudinal member (e.g., a suture, a string, or a rope comprising a metal or a fabric) may be coupled to implants 7040.

Implant 7040 comprises a proximal coil at a proximal end thereof, a distal coil at a distal end thereof, and, typically, a plurality of successive contiguous coils disposed between the proximal and distal coils. The distal coil of implant 7040 comprises a pointed tip which punctures tissue of urethra 60 and prostate 100 during implantation of implant 7040. Ultimately, both the distal and proximal coils are disposed entirely within prostate tissue of a patient. That is, the distal coil does not extend beyond the prostate capsule (the capsule that surrounds the prostate), and the proximal coil does not extend into the urethra. Optionally, the coiled implant facilitates pinching of tissue of the patient between the successive coils of implant 7040 during implantation thereof, thus supplementing compression of prostate tissue 100.

As shown in FIGS. 27A-D, implants 7040 are coupled to wire 7010 extending from one coiled implant to the next coiled implant. Typically, delivery tool 7021 houses at least one set of successive coiled implants 7040, e.g., three coiled implants, comprising a wire 7010 extending between implants 7040. Wire 7010 is typically flexible and comprises a biocompatible material.

Reference is made to FIG. 27A. Delivery tool 7021 is inserted into urethra 60 of penis 160 of the patient and is advanced distally toward bladder 80 of the patient as described herein with reference to FIGS. 21A-F.

Delivery tool 7021, which houses at least one set of coiled implants 7040 which are coupled to wire 7010, optionally has a deflectable tip 7026 having an open distal end. (Alternatively or additionally, delivery tool comprises one or more inflatable elements, as described hereinabove with reference to FIGS. 25A and 25B.) When the distal end of the delivery tool reaches a portion of urethra 60 at prostate 100 that is constricted due to pressure exerted thereupon by prostate 100, the deflectable tip is steered radially away from a longitudinal axis of the delivery tool. In response to the deflecting, the distal tip of tool 7021 pushes the wall of the urethra, which compresses tissue outside of the urethra, i.e., prostate tissue, and consequently the perimeter of the urethra at the prostate expands. Delivery tool 7021 then delivers first coiled implant 7040 into the portion of the tissue of the prostate that has been compressed, and the implant functions to maintain the tissue in a compressed state upon withdrawal of the delivery tool from the urethra.

FIG. 27A shows deflectable portion 7026 of the delivery tool optionally having returned to a position that is aligned with respect to the longitudinal axis of the tool 7021, following implantation of implant 7040. As shown, even after the distal tip of deflectable portion 7026 has been moved away from the wall of urethra 60, implant 7040 maintains the tissue of prostate 100 in a compressed state. This creates an enlarged perimeter of urethra 60 in the vicinity of implant 7040, as shown. Wire 7010 is coupled to the proximal end of first implant 7040 and to the proximal end of a second implant 7040 (not shown) which is still disposed within delivery tool 7021.

As shown in FIG. 27B, second implant 7040 is implanted in tissue of prostate 100 in a vicinity of prostate 100 that is adjacent to the site of implantation of first implant 7040. The steps for deflection of delivery tool 7021, rotation of knob 7076 and implantation of implants 7040 are repeated as described herein with reference to FIGS. 21A-F, and second implant 7040 is implanted in tissue of prostate 100. As shown, wire 7010 is coupled to the proximal ends of both implants 7040 and extends between the proximal ends of the implants.

FIG. 27C shows deflectable portion 7026 of the delivery tool 7021 optionally having returned to a position that is aligned with respect to the longitudinal axis of the tool, following implantation of second implant 7040. As shown in FIG. 27C, wire 7010 enhances the enlargement of the perimeter of urethra 60, and subsequently helps to maintain the enlarged perimeter of urethra 60 in the area 7015 that is between the sites of implantation of the implants 7040. Over the long term (e.g., months or years), wire 7010 reduces constriction of the urethra in areas 7015 that would otherwise occur due to pressure exerted thereupon by prostate 100. Additionally, wire 7010 typically helps maintain implants 7040 in place.

Reference is made to FIG. 27D, which shows a first set 7050 of three coiled implants 7040 (by way of illustration and not limitation), coupled to wire 7010, implanted in prostate tissue 100. Additionally, FIG. 27D shows a second set 7052 of three coiled implants 7040 coupled to wire 7010 being implanted in tissue of the prostate in a vicinity of the prostate that is opposite the site of implantation of the first set 7050 of implants 7040. As shown, a generally even enlargement of the perimeter of urethra 60 is obtained along a longitudinal axis of the urethra, both at the vicinity of implants 7040 and in areas 7015 which are between the sites of implantation of implants 7040.

It is to be noted that any suitable number of implants 7040, e.g., between 2 and 9 implants, may be implanted in tissue of prostate 100. Typically, delivery tool 7021 implants implants 7040 by orienting the implants radially with respect to the urethra.

Additionally, it is to be noted that, although coiled implants 7040 are implanted around urethra 60 symmetrically with respect to each other, as shown in FIG. 27, implants 7040 may be positioned at any suitable locations in prostate 100 and at any desired angle with respect to the longitudinal axis of urethra 60.

Reference is made to FIGS. 28A-D, which are schematic illustrations of a system 8020 comprising at least one implant rod 8080, at least one coiled implant 8040, and an implant-delivery tool 8021 for delivering the coiled implant, in accordance with an application of the present invention. A rod-delivery tool (not shown) is inserted into urethra 60 of penis 160 of a patient and is advanced distally toward bladder 80 of the patient. The rod-delivery tool, which houses a plurality of rods 8080, e.g., two, has a deflectable tip having an open distal end. When the distal end of the rod-delivery tool is disposed inside bladder 80, the deflectable tip is steered radially, e.g., by 90 degrees, away from a longitudinal axis of the rod-delivery tool such that the distal end is generally perpendicular to the longitudinal axis of the rod-delivery tool. The deflectable tip is then steered again until it reaches a position in which it has turned 180 degrees, such that the open distal end of the deflectable tip is in a vicinity of prostate 100 and is facing the prostate. Rod 8080 is then advanced through the bladder wall and disposed in the prostate such that the longitudinal axis of the rod is parallel to the longitudinal axis of the urethra. Rod 8080 typically is shaped to define a pointed tip which punctures tissue of the bladder wall and prostate 100 during implantation of the rod.

For some applications, following implantation of first rod 8080, the distal end of the rod-delivery tool remains within bladder 80, and a second rod 8080 is implanted in tissue of prostate 100. Second rod 8080 is typically implanted in a vicinity of prostate 100 that is opposite the site of implantation of the first rod. (Alternatively, if for example three rods are used, then they are typically separated by about 120 degrees.) Prior to implantation, the second rod is advanced distally within the rod-delivery tool to a position at the distal tip of the deflectable portion, such that rod 8080 is primed for implantation. Once the rod is positioned at the distal tip of the deflectable portion, second rod 8080 is advanced through the bladder wall and disposed in the prostate, using the technique described hereinabove with respect to first rod 8080, such that the longitudinal axis of the rod is parallel to the longitudinal axis of the urethra.

Alternatively, rods 8080 are implanted in tissue of prostate 100 by any other suitable implantation procedure, e.g., using a hollow needle.

Rod 8080 is flexible enough to be maneuvered through the rod-delivery tool and is generally rigid enough in order to support tissue of the prostate. Rod 8080 typically comprises a biocompatible material configured for permanent implantation in the prostate. Rod 8080 has proximal and distal ends, and an elongated cylindrical body extending between the proximal and the distal ends. Typically, as rod 8080 is advanced through tissue of prostate 100, tissue of prostate 100 applies a frictional force to the rod. For some applications, in order to reduce the effect of the frictional force applied to rod 8080, the rod is coated with a low-friction coating, e.g., PTFE (Teflon), MoST, ADLC or the like. For some applications, the rod surface is polished, e.g., electro-polished, mechanically polished, or otherwise, to reduce friction as rod 8080 is advanced through the tissue of the patient.

It is to be noted that for some embodiments, a single delivery tool is used to implant both rods 8080 and implants 8040.

FIG. 28A shows rods 8080 implanted in tissue of prostate 100, on opposite sides of prostate 100 around urethra 60. The rods are placed in a position in which their longitudinal axes are parallel to the longitudinal axis of urethra 60. It is to be noted, however, that rods 8080 may be positioned at any suitable location in prostate 100 and at any desired angle with respect to the longitudinal axis of urethra 60. Typically, the rods are configured to be disposed at an angle that is less than 90 degrees with respect to the longitudinal axis of urethra 60, e.g., less than 30 degrees. For some embodiments, rods 8080 are implanted substantially in parallel with the longitudinal axis of the urethra. As shown in FIG. 28A, implant-delivery tool 8021, housing at least one coiled implant 8040 (not shown), is about to be inserted into urethra 60 of a penis 160 of a patient. Typically, tool 8021 is preloaded with a plurality of successively disposed coiled implants 8040, which are designated for implantation at least in part around rods 8080 in tissue of prostate 100.

Reference is made to FIG. 28B-C. Implant 8040 comprises a coiled implant comprising a proximal coil at a proximal end of implant 8040, a distal coil at a distal end of implant 8040, and typically a plurality of successive contiguous coils disposed between the proximal and distal coils. The distal coil of implant 8040 comprises a pointed tip which punctures tissue of urethra 60 and prostate 100 during implantation of implant 8040. The proximal coil of implant 8040 is shaped to define a hook. For some applications, during implantation of implant 8040 the proximal coil is wound around rod 8080 that is implanted in tissue of prostate 100, as shown in FIG. 28C. Ultimately, both the distal and proximal coils are disposed entirely within prostate tissue of a patient. That is, the distal coil does not extend beyond the prostate capsule (the capsule that surrounds the prostate), and the proximal coil does not extend into the urethra.

Reference is still made to FIGS. 28B-C. As shown, delivery tool 8021 is inserted into urethra 60 of a penis 160 of the patient and is advanced distally toward bladder 80 of the patient. The steps for deflection of tool 8021 and implantation of implant 8040 are carried out as described in FIGS. 21A-F. Ultimately, implant 8040 is implanted such that the proximal end of coiled implant 8040 is wound around rod 8080, coupling the implant to the rod and securing it in place. For some applications, delivery tool 8021 comprises a screwdriver tool (not shown) which facilitates implantation of coiled implant 8080 in tissue of prostate 100 such that the proximal end of implant 8040 is wound around rod 8080. As shown, knob 8076 of tool 8021 is rotated by the operating physician, in the direction as indicated by arrow 8033A, in order to rotate the screwdriver tool and thereby effect implantation of implant 8040 around rod 8080. Following the corkscrewing of implant 8040 in tissue of prostate 100, implant 8040 is decoupled from the screwdriver tool and from delivery tool 8021 and maintains the tissue in a compressed state in order to enlarge the perimeter of urethra 60 in the vicinity of implant 8040.

FIG. 28C shows implantation of second coiled implant 8040 in tissue of prostate 100 in a vicinity of the prostate that is opposite the site of implantation of first implant 8040. The steps for implantation of second implant are carried out as described with reference to implantation of first implant 8040. As shown with reference to first implant 8040, even after the distal tip of deflectable portion 8026 of delivery tool 8021 has been moved away from the wall of urethra 60, implant 8040 maintains the tissue of prostate 100 in a compressed state. This creates an enlarged perimeter of urethra 60 in the vicinity of implant 8040, as shown. Optionally, the coiled implant facilitates pinching of tissue of the patient between the successive coils of implant 8040 during implantation thereof, thus supplementing compression of prostate tissue 100. Once implanted in place, implants 8040 pull on rods 8080 causing rods 8080, which are positioned generally parallel to the longitudinal axis of the urethra, to shift positions such that the rods are at an angle that is typically less than 90 degrees with respect to the longitudinal axis of urethra 60, e.g., less than 30 degrees. Optionally, the shifting in position of the rods is also due to pinching of tissue of the prostate between the successive coils of implant 8040 during implantation thereof.

Reference is made to FIG. 28D, which shows 2 pairs of coiled implants 8040, by way of illustration and not limitation, implanted in tissue of prostate 100, on opposite sides of urethra 60. The proximal ends of implants 8040 are wound around rods 8080 (by way of illustration and not limitation, i.e., any portion of implant 8040 any portion of may be wound around rod 8080). Implants 8040 maintain the tissue of prostate 100 in a compressed state. This creates an enlarged perimeter of urethra 60 in the vicinity of implant 8040, as shown. Additionally, as shown, an enlarged perimeter of urethra 60 is also obtained in the area 8015 that is between the each pair of implants 8040. This is due to additional pulling/tension effect of implants 8040 on tissue of prostate 100, when implants 8040 are coupled to rods 8080. For some application, rod 8080 may enable the use of a reduced number of implants 8040.

It is to be noted that the prostatic implants described herein may be coated with any low-friction coating as described herein. For example, the implants may be coated with a substance, such as but not limited to, a medication (e.g., an antibiotic) or with an electrical insulator (e.g., Teflon). The implants may be coated with low friction coatings, e.g., PTFE (Teflon), MoST, ADLC or the like. In some embodiments, the surfaces of the implants are polished, e.g., electro-polished, mechanically polished, or other, to reduce friction as the implants are implanted in the tissue of the patient. In some embodiments, a portion of each of the implants, i.e., one or more of the coils, may be energized to deliver RF energy, for example, to ablate tissue. In some embodiments, a portion of each one of the implants is coupled to an electrode. Additionally or alternatively, the portion of each implant may be energized to provide ultrasound or thermal energy (e.g., heating or cooling).

It is to be further noted that the prostatic implants described herein are selected to provide a length according to the needs of a given patient. A length of prostate 100 is measured prior to the implantation procedure such that an implant of a suitable length is selected. Typically, the end-to-end length of the coiled implant ranges from between 2.5 cm and 7 cm, to accommodate a prostate length of between 3 and 8.6 cm, respectively.

The scope of the present invention includes application of the techniques described herein to body lumens other than the urethra, in order to treat a condition of patient. For example, the implants described herein may be sized for implantation around another body lumen of the patient, such as the esophagus or a blood vessel which is connected to a body cavity.

The scope of the present invention includes embodiments described in the following patents and patent applications, which are incorporated herein by reference.

In an embodiment, techniques and apparatus described in one or more of the following patents and patent applications are combined with techniques and apparatus described herein:

U.S. patent application Ser. No. 11/325,731 to Gross, entitled, “Implant and delivery tool therefor,” filed Jan. 5, 2006;

U.S. Provisional Patent Application 60/930,705 to Gross et al., entitled, “Prostate implant and methods for insertion and extraction thereof,” filed May 18, 2007;

PCT Patent Application PCT/IL08/00677 to Gross et al., entitled, “Prostate implant and methods for insertion and extraction thereof,” filed May 18, 2008; and/or

U.S. Provisional Patent Application 61/200,372 to Gross et al., entitled, “Intraurethral and extraurethral apparatus,” filed Nov. 26, 2008.

For some applications, techniques described herein are practiced in combination with techniques described in one or more of the references cited in the Background and Cross-References section of the present patent application, which are incorporated herein by reference.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus, comprising:
an implant; and
a delivery tool, removably coupled to the implant, the tool configured to:
advance the implant distally through a urethra of a patient until the implant emerges at a distal end of the urethra into a bladder of the patient, and
subsequently, facilitate expansion of the urethra by retracting the implant and by the retracting, implant the implant around the urethra in tissue of a prostate of the patient.

2-4. (canceled)

5. The apparatus according to claim 1, wherein the implant comprises a transurethrally-implantable prostatic implant configured to be positionable in the prostate of the patient, and wherein the implant is shaped so as to define an implant lumen that surrounds an outer circumference of the urethra upon implantation.

6-8. (canceled)

9. The apparatus according to claim 1, wherein the implant is configured to treat benign prostate hyperplasia.

10-17. (canceled)

18. The apparatus according to claim 1, wherein the implant comprises at least one rod, and wherein the delivery tool is configured to implant the rod in the tissue of the prostate at an angle that is less than 90 degrees with respect to a longitudinal axis of the urethra.

19. The apparatus according to claim 18, further comprising at least one coiled implant, wherein the delivery tool is configured to implant the implant in the tissue of the prostate in a manner in which the at least one coiled implant is couplable to the rod at at least a portion of the coiled implant.

20. The apparatus according to claim 19, wherein the rod has a longitudinal axis that is less than 90 degrees with respect to the longitudinal axis of the urethra, and wherein the coiled implant is implantable at a non-zero angle with respect to the longitudinal axis of the rod.

21. The apparatus according to claim 1, wherein the implant comprises a coiled implant comprising at least one coil.

22. The apparatus according to claim 21, wherein the delivery tool is configured to corkscrew the coiled implant into the prostate while retracting the implant.

23-50. (canceled)

51. A method, comprising:
distally advancing an implant through a urethra of a patient until the implant emerges in a bladder of the patient; and
facilitating expanding of a pre-operative perimeter of a portion of the urethra to a post-operative perimeter of the portion of the urethra that is larger than the pre-operative perimeter by proximally retracting the implant and implanting the implant in prostate tissue surrounding the urethra.

52. The method according to claim 51, wherein expanding the pre-operative perimeter comprises treating benign prostate hyperplasia.

53. The method according to claim 51, wherein advancing the implant comprises advancing a coiled implant defining an inner lumen thereof, and wherein implanting the implant comprises surrounding a portion of the urethra by the inner lumen of the coiled implant.

54. The method according to claim 53, wherein facilitating the expanding of the pre-operative perimeter of a portion of the urethra comprises the surrounding of the portion of the urethra by the inner lumen of the coiled implant.

55. The method according to claim 51, wherein advancing the implant comprises advancing at least one rod through the urethra, and wherein implanting the implant comprises retracting the rod into the prostate tissue at an angle that is less than 90 degrees with respect to a longitudinal axis of the urethra.

56. The method according to claim 55, further comprising:
distally advancing at least one coiled implant through the urethra of the patient;
further facilitating expanding of the pre-operative perimeter of the portion of the urethra by implanting the at least one coiled implant in the prostate tissue; and
facilitating coupling to the rod at least a portion of the coiled implant.

57. The method according to claim 56, wherein the rod has a longitudinal axis that is less than 90 degrees with respect to the longitudinal axis of the urethra, and wherein implanting the at least one coiled implant comprises implanting the at least one coiled implant at a non-zero angle with respect to the longitudinal axis of the rod.

58. The method according to claim 51, wherein the implant includes a radially-expandable implant, and wherein advancing the implant into the bladder comprises facilitating the expansion of the implant within the bladder of the patient.

59. The method according to claim 51, wherein the implant includes a conically-shaped coiled implant in which a diameter of a proximal coil thereof is larger than a diameter of a distal coil thereof, and wherein implanting the implant comprises implanting the conically-shaped coiled implant in the prostate tissue of the patient.

60-61. (canceled)

62. The method according to claim 51, wherein proximally retracting the implant comprises corkscrewing the implant into the prostate tissue by rotating at least a portion of the implant.

63-375. (canceled)
